SEQUENCE LISTING (244 sequences)

SEQ ID NO: 1 - protein, 477 aa, Lactobacillus reuteri
  MISC_FEATURE (1)..(477): DSM 20016
MQINDIESAVRKILAEELDNASSSSANVAATTDNGHRGIFTNVNDAIAAAKAAQEIYRDKPIAVRQQVIDAIKEGFRPYIEKMAKDIKEETGMGTVEAKIAKLNNALYNTPGPEILEPVVENGDGGMVMYERLPYGVIGAVGPSTNPSETVIANAIMMLAGGNTLYFGAHPGAKNVTRWTIEKMNDFIADATGLHNLVVSIETPTIESVQQMMKHPDIAMLAVTGGPAVVHQAMTSGKKAVGAGPGNPPAMVDATADIDLAAHNIITSASFDNDILCTAEKEVVAESSIKDELIRKMQDEGAFVVNREQADKLADMCIQENGAPDRKFVGKDATYILDQANIPYTGHPVEIICELPKEHPLVMTEMLMPILPVVSCPTFDDVLKTAVEVEKGNHHTATIHSNNLKHINNAAHRMQCSIFVVNGPSYVGTGVADNGAHSGASALTIATPTGEGTCTARTFTRRVRLNSPQGFSVRNWY

SEQ ID NO: 2 - protein, 477 aa, Lactobacillus brevis
  MISC_FEATURE (1)..(477): ATCC 367
MNTENIEQAIRKILSEELSNPQSSTATNTTVPGKNGIFKTVNEAIAATKAAQENYADQPISVRNKVIDAIREGFRPYIEDMAKRIHDETGMGTVSAKIAKLNNALYNTPGPEILQPEAETGDGGLVMYEYAPFGVIGAVGPSTNPSETVIANAIMMLAGGNTLFFGAHPGAKNITRWTIEKLNELVADATGLHNLVVSLETPSIESVQEVMQHPDVAMLSITGGPAVVHQALISGKKAVGAGAGNPPAMVDATANIALAAHNIVDSAAFDNNILCTAEKEVVVEAAVKDELIMRMQQEGAFLVTDSADIEKLAQMTIGPKGAPDRKFVGKDATYILDQAGISYTGTPTLIILEAAKDHPLVTTEMLMPILPVVCCPDFDSVLATATEVEGGLHHTASIHSENLPHINKAAHRLNTSIFVVNGPTYCGTGVATNGAHSGASALTIATPTGEGTATSKTYTRRRRLNSPEGFSLRTWEA

SEQ ID NO: 3 - protein, 477 aa, Pediococcus acidilactici
MEIQNLEEDIRRILSEELKKSGTSQTASTSDAGQNGIFKTVDEAIAAAKAAEDVYIDKPLAFREKVLTAIREGFRPYIEKMAKDIKDETGMGTVEAKIAKLNNALYNTPGTEILQPEAETGDGGLVMYEYAPFGVIGAVGPSTNPSETVIANAIMMLAGGNTLYFGAHPGAKKITRWTIEKLNKLVYEATGMKNLVVSIEEPSIESVQEMMQHPDIAMLSITGGPAVVHQALVSGKKAVGAGAGNPPAIVDATANVALAAHNIVDSASFDNNILCTAEKEVVVESSVKDELIKKMQEEGAFLVTNASDIDKLAEMTIGKNGAPDRQFVGKDATYILDKAGIAYTGTPKLIIMEAQKDHPLVTTEMLMPIVPVVSCPTFDQVLATAVEVEQGLHHTASIHSENLPNINRAAHRMNTSIFVVNGATYVGTGVGANGAHAGASALTIATPTGEGTATAKTFTRRRRLNSPEAFSLRSWEA

SEQ ID NO: 4 - protein, 476 aa, Pediococcus claussenii
  MISC_FEATURE (1)..(476): ATCC BAA-344
MEMDKLEQDIRRILSEELQDSDNSVSASSDNGTNGIFKTVDEAIAAAKAAQEIYVDKSLAFRNQVLDAIKEGFRPYIEQMAKDIKEETGMGTVEAKIAKLNNALYNTPGTEILEPEAETGDGGLVLYEYAPFGVIGAVGPSTNPSETVIANALMMLAGGNTVYFGAHPGAKKITRWTIEKLNEFVFKATGMRNMVVSIEEPSIESVQQMMQHPDIAMLSITGGPGVVHQAMISGKKAVGAGAGNPPAIVDATANIDLAAHNIVDSSSFDNNILCTAEKEVVVEESVKDELISKMQNEGAFLVTSAHDIEKIVQITIGKNGAPDRKFVGKDATFILDSAGINYTGTPKLIILEAHKNHPLVTTEMLMPILPVVSCPTFDRALATAVEVEQGLHHTASIHSENLPHINQAAHRMNTSIFVVNGATYVGTGVGANGAHAGASALTIATPTGEGTATAKTFTRRRRLNSPEAFSLRSWEA

SEQ ID NO: 5 - protein, 481 aa, Lactobacillus collinoides
MADQNIEAEIRRILQEELSGNASSSAAGTTTSQPDGLGNRIFTNVNDAIAAAKQAQAIYQDKPLAFRKKVVQAIKDGFGPYIEYMAKQTREETGMGTAEAKIAKLKNALYNTPGVELLDPEVETGDGGMVMYEYTPFGVIGAVGPSTNPCETVLNNSIMMMSAGNALFFGAHPGAKNITRWAVEKLNEFVYKATGLKNLLVSLDTPSIESVQEMMQHPDVAMLAVTGGPAVVHQALTSGKKAVGAGAGNPPAMVDATADIDLAAHNLFTSAKFDNEILCTSEKEIIAEDSIKDELLQKIVAKGACLVTDPKDIKHLADMTIGDNGAPDRKYVGKDATVILDAAGISYTGDPKLIMMDVDKDNPLVKTEMLMPILPIVGCPDFDAVLATAIEVEGGNHHTASIHSNNILHINKAAHRMNTSIFVANGPTFAATGVGDNGYYSGAAALTIATPTGEGTTTTKTFTRRRRFNCPQGFSLRSWEV

SEQ ID NO: 6 - protein, 469 aa, Listeria welshimeri
  MISC_FEATURE (1)..(469): serovar 6b str. SLCC5334
MESLELEQLVKKVLLEKLAEQKDVPVKTTTQGAKSGIFDTVDEAVQAAVQAQNSYKEKSLEERRNVVKAIREALYPEIESIATRAVAETGMGNVTDKILKNTLAIEKTPGVEDLYTEVATGDNGMTLYELSPYGVIGAVAPSTNPTETLICNTIGMLAAGNAVFYSPHPGAKNISLWLIEKLNTIVRESCGIDNLVVTVEKPSIQAAQEMMNHPKVPLLVITGGPGVVLQAMQSGKKVIGAGAGNPPSIVDETANIEKAAADIVDGASFDHNILCIAEKSVVAVDSITDFLLFQMEKNGALHVTNPSDIKKLEKVAVTDKGVTNKKLVGKSASEILKEAGITCDFTPRLIIVETDKSHPFATVELLMPIVPVVRVPDFDEALKVAIELEQGLHHTATMHSQNISRLNKAARDMQTSIFVKNGPSFAGLGFRGEGSTTFTIATPTGEGTTTARHFARRRRCVLTDGFSIR

SEQ ID NO: 7 - protein, 469 aa, Listeria innocua
  MISC_FEATURE (1)..(469): Clip11262
MESLELEQLVKKVLLEKLAEQKEVPTKTTTQGAKSGVFDTVDEAVQAAVIAQNCYKEKSLEERRNVVKAIREALYPEIETIATRAVAETGMGNVTDKILKNTLAIEKTPGVEDLYTEVATGDNGMTLYELSPYGVIGAVAPSTNPTETLICNSIGMLAAGNAVFYSPHPGAKNISLWLIEKLNTIVRDSCGIDNLIVTVAKPSIQAAQEMMNHPKVPLLVITGGPGVVLQAMQSGKKVIGAGAGNPPSIVDETANIEKAAADIVDGASFDHNILCIAEKSVVAVDSIADFLLFQMEKNGALHVTNPSDIQKLEKVAVTDKGVTNKKLVGKSATEILKEAGIACDFTPRLIIVETEKSHPFATVELLMPIVPVVRVPDFDEALEVAIELEQGLHHTATMHSQNISRLNKAARDMQTSIFVKNGPSFAGLGFRGEGSTTFTIATPTGEGTTTARHFARRRRCVLTDGFSIR

SEQ ID NO: 8 - protein, 469 aa, Listeria monocytogenes
  MISC_FEATURE (1)..(469): ATCC 19117
MESLELEQLVKKVLLEKLAEQKDAPVKTTVKGAKSGVFDTVDEAVQAAVIAQNNYKEKSLEERRNVVKAIREALYPEIESIAARAVAETGMGNVADKILKNTLAIEKTPGVEDLYTEVATGDNGMTLYELSPYGVIGAVAPSTNPTETLICNTIGMLAAGNAVFYSPHPGAKNISLWLIEKLNTIVRESCGIDNLVVTVEKPSIQAAQEMMNHPKVPLLVITGGPGVVLQAMQSGKKVIGAGAGNPPSIVDETANIEKAAADIVDGASFDHNILCIAEKSIVAVDSIADFLMFQMEKNGALHVTNPSDIQKLEKVAVTDKGVTNKKLVGKSASEILKEAGIVCDFSPRLIIVETEKTHPFATVELLMPIVPVVRVPNFDEALDVAIELEQGLHHTATMHSQNISRLNKAARDMQTSIFVKNGPSFAGLGFRGEGSTTFTIATPTGEGTTTARHFARRRRCVLTDGFSIR

SEQ ID NO: 9 - protein, 469 aa, Listeria marthii
  MISC_FEATURE (1)..(469): FSL S4-120
MESLELEQLVKKVLLEKLAEQKEAPAKPITQGAKSGIFDTVDEAVQAAVIAQNCYKEKSLEERRNVVKAIRETLYPEIETIATKAVAETGMGNVADKILKNTLAIEKTPGVEDLYTEVATGDNGMTLYELSPYGVIGAVAPSTNPTETLICNTIGMLAAGNAVFYSPHPGAKNISLWLIEKLNTIVRESCGIDNLVVTVEKPSIQAAQEMMNHPKVPLLVITGGPGVVLQAMQSGKKVIGAGAGNPPSIVDETANIEKAAADIVDGASFDHNILCIAEKSIVAVESIADFLLFQMEKNGALHVTNPSDIQKLEKVAVTDKGVTNKKLVGKSAAEILKEAGITCDFTPRLIIVETTKTHPFATVELLMPIVPLVRVPDFDEALEVAIELEQGLHHTATMHSQNISRLNKAARDMQTSIFVKNGPSFAGLGFRGEGSTTFTIATPTGEGTTTARHFARRRRCVLTDGFSIR

SEQ ID NO: 10 - protein, 469 aa, Listeria ivanovii
  MISC_FEATURE (1)..(469): subsp. ivanovii PAM 55
MESLELEQLVKKVLLEKLAGQNEETPKKPSQGAKSGIFDTVDEAVQAAVIAQNCYKEKSLEDRRNVVKAIREALYPEIENIATRAAAETGMGNVADKILKNTLAIEKTPGVEDLYTEVATGDNGMTLYELSPYGVIGAVAPSTNPTETLICNTIGMLAAGNAVFYSPHPGAKNISLWLIEKLNTIVRESCGIDNLVVTVEKPSIQAAQEMMNHPKVPLLVITGGPGVVLQAMQSGKKVIGAGAGNPPSIVDETANIEKAAADIVAGASFDHNILCIAEKSVVAVDSITDFLLFQMEKNGAFHVTNPSDIRKLEKVAVTEKGVTNKKLVGKSASEILKEAGIACDFTPRLIIAETDRSHPFATVELLMPIVPVVRVADFDQALEVALELEQGLHHTATMHSQNISRLNKAARDMQTSIFVKNGPSFAGLGFGGEGSATFTIATPTGEGTTTARHFARRRRCVLTDGFSIR

SEQ ID NO: 11 - protein, 469 aa, Listeria seeligeri
  MISC_FEATURE (1)..(469): serovar 1/2b str. SLCC3954
MESLELEQLVKKVLLEKLAGQNEETPKKPSQGAKSGIFDTVDEAVQAAVIAQNCYKEKSLEDRRNVVKAIREALYPEIKNIATRAVAETGMGNVADKILKNTLAIEKTPGVEDLYTEVATGDNGMTLYELSPYGVIGAVAPSTNPTETLICNTIGMLAAGNAVFYSPHPGAKNISLWLIEKLNTIVRESCGIDNLVVTVEKPSIQAAQEMMNHPKVPLLVITGGPGVVLQAMQSGKKVIGAGAGNPPSIVDETANIEKAAADIVAGASFDHNILCIAEKSVVAVDSITDFLLFQMEKNGALHVTNPSDIRKLEKVAVTEKGVTNKKLVGKSASEILKEAGIACDFTPRLIIVETDRSHPFATVELLMPIVPVVRVADFDQALEVALELEQGLHHTATMHSQNISRLNKAARDMQTSIFVKNGPSFAGLGFGGEGSATFTIATPTGEGTTTARHFARRRRCVLTDGFSIR

SEQ ID NO: 12 - protein, 464 aa, Shewanella putrefaciens
  MISC_FEATURE (1)..(464): CN-32
MNTTELENMIRNILADNLKGTATAPGNIQHTIFARVEDAITASYDAYKKYMAEPLALRTRIITALKEELAPWIKEMSERAAEETGMGNAPDKISKNTAALNNTPGIEDLTTSALTGDGGMVLFELSPFGVIGAIAPSTNPTETIINNTISMLAAGNAVYFSPHPGAKKVSLWLIEKIEDIIYRVSGIRNLVTTVAEPTFDATREMMSDPRIALLAVTGGPAIVNMAMKTGKKVIGAGPGNPPVLVDETACPVKAAKDIVDGASFDHNVLCIAEKCVIVVDSIADRLMDNMQKNDAFLVKTPGDIARLRKVVINDKGEANKKLVGKSPAVILQAADLNTSTAPRLIIVEVEQDDPLVMVEQLMPVLPVVRVSDFETGLALALKVENEQHHTAIMHSQNVTRLNLAAKTMQTSIFVKNGPSYAGLGIGAEGFTTFTIATPTGEGTTSARSFARKRRCVLTNGFSIR

SEQ ID NO: 13 - protein, 464 aa, Kosakonia radicincitans
  MISC_FEATURE (1)..(464): DSM 16656
MNTTELENMIRTILADNLTGIATAPGNIQHTIFARVEDAITASYDAYKKYLAEPLALRTRIITALKEELAPWIKEMSERAAEETGMGNALDKISKNTAALNNTPGIEDLTTSALTGDGGMVLFELSPFGVIGAIAPSTNPTETIINNTISMLAAGNAVYFSPHPGAKKVSLWLIEKIEDIIYRVSGIRNLVTTVAEPTFDATREMMSDPRIALLVVTGGPAIVNMAMKTGKKVIGAGPGNPPVLVDETACPVKAAKDIVDGASFDHNVLCIAEKCVIVVDSIADRLVENMQKNDAFLVKTPGDIARLRQVVINDKGEANKKLVGKSPAVILQAADLNTSTAPRLIIVEVEQDDPLVMVEQLMPVLPVVRVRDFETGLALALKVENDQHHTAIMHSQNVSRLNLAAKTMQTSIFVKNGPSYAGLGIEAEGFTTFTIATPTGEGTTSARSFARKRRCVLTNGFSIR

SEQ ID NO: 14 - protein, 467 aa, Tolumonas auensis
  MISC_FEATURE (1)..(467): DSM 9187
MNNTELESLIRTILTEQLTPSATDTPACTASSVALFDDVDSAICAAHAAFLRYQEAPLKTRSAIIAAIRAEIAPCLSELAERAAAETGMGNTADKILKNKAALENTPGIEDLKTTALTGDEGMVLFEYSPFGVVGAVAPSTNPTETIINNSISMLAAGNAIYFSPHPGAKNISLWLIQKMEEIAFKVCGIHNLIVTVKEPTFEATQQMMAHDKIALLAITGGPGIVNMGLKSGKKVIGAGAGNPPCLVDETAEIVKAAQDIVAGASFDYNLPCIAEKSVIAVDCIADQLIQQMREFGAMQITDPQQIAQLREVCIQKGAANKSLVGKSPATILAAAGIPCPAKEPRLIILEVPANDPFVVTEQLMPVLPIVRVDNFEQGLQLALKVEDGLHHTAMMHSQNVSRLNKAAHLMQTSIFVKNGPSYAGIGVGAEGFTTFTIATPTGEGTTSARTFGRLRRCVLTNGFSIR

SEQ ID NO: 15 - protein, 461 aa, Citrobacter koseri
  MISC_FEATURE (1)..(461): ATCC BAA-895
MNTSELETLIRNILSEQLAPAQAETQGHGIFQSVGEAIDAAHQAFLRYQQCPLKTRSAIISALRQELTPHLATLAAESAAETGMGNKEDKFLKNKAALDNTPGIEDLTTTALTGDGGMVLFEYSPFGVIGSVAPSTNPTETIINNSISMLAAGNSVYFSPHPGAKNVSLKLIGMIEDIAFRCCGIRNLVVTVAEPTFEATQQMMAHPNIAVLAITGGPGIVAMGMKSGKKVIGAGAGNPPCIVDETADIVKAAEDIINGAAFDYNLPCIAEKSLIVVESVAERLVQQMQAFGALLLNAADIDKLRAVCLPEGHANKKLVGKSPAAMLEAAGIAVPAKPPRLLIGIVSADDPWVTSEQLMPMLPVVKVDNFDSALALALKVEEGLHHTAIMHSQNVSRLNLAARTLQTSIFVKNGPSYAGIGVGGEGFTTFTIATPTGEGTTSARTFARSRRCVLTNGFSIR

SEQ ID NO: 16 - protein, 462 aa, Yersinia enterocolitica
  MISC_FEATURE (1)..(462): subsp. enterocolitica 8081
MNTNDLESLIRTILTEQLTPVTAPASSAIFASVDEAINAAHSAFLRYQQSPMKTRSAIIRAIREQLKPQLVSLSERGASETGMGNKEDKFLKNKAALENTPGIEDLSTTALTGDGGMVLFEYSPFGVIGSVTPSTNPTETIINNSISMLAAGNAVYFSPHPGAKAVSLDLIAQIEEIIFNSCGIRNLVVTVKEPSFEATQQMMAHDKIALLAITGGPAIVAMSMKSGKKVIGAGAGNPPCLVDETAELVKAAQDIVAGASFDYNLPCIAEKSLIVVESVADRLLQQMQAFDALLISNPQEIDSLRKACLTPQGHANKNLVGKSPIELLKAAGITCPAKAPRLLLVEVAGDDPLVTTEQLMPLLPVVRVKDFDAALTLALHVEGGLHHTATMHSQNVSRLNLAARLLQTSIFVKNGPSYAGIGVGGEGFTTFTIATPTGEGTTSARTFARQRRCVLTNGFSIR

SEQ ID NO: 17 - protein, 464 aa, Salmonella enterica
  MISC_FEATURE (1)..(464): subsp. enterica serovar Mbandaka str. ATCC 51958
MNTSELETLIRTILSEQLTTPAQTTAQPQGKGIFQSVSEAIDAAHQAFLRYQQCPLKTRSAIISAMRQELTPLLATLAEESANETGMGNKEDKLLKNKAALDNTPGVEDLTTTALTGDGGMVLFEYSPFGVIGSVAPSTNPTETIINNSISMLAAGNSVYFSPHPGAKKVSLKLISLIEEIAFRCCGIRNLVVTVAEPTFEATQQMMAHPRIAVLAITGGPGIVAMGMKSGKKVIGAGAGNPPCIVDETADLVKAAEDIINGASFDYNLPCIAEKSLIVVESVAERLVQQMQTFGALLLSPADTDKLRAVCLPEGQANKKLVGKSPSAMLEAAGIAVPAKAPRLLIALVSADDPWVTSEQLMPMLPVVKVSDFDSALALALKVEEGLHHTAIMHSQNVSRLNLAARTLQTSIFVKNGPSYAGIGVGGEGFTTFTIATPTGEGTTSARTFARSRRCVLTNGFSIR

SEQ ID NO: 18 - protein, 462 aa, Yersinia mollaretii
  MISC_FEATURE (1)..(462): ATCC 43969
MNTHDIESLIRTILTEQLTPATASAVSAIFASVDEAVTAAHSAFLRYQQSPMKTRSAIISALREQLAPQLASLSERGASETGMGNKEDKFLKNRAALENTPGIEDLSTTALTGDGGMVLFEYSPFGVIGSVAPSTNPTETIINNSISMLAAGNAVYFSPHPGAKAVSLDLIAQIEAIIFNRCGIRNLVVTVQEPSFEATQQMMAHDKIALLAITGGPAIVAMGMKSGKKVIGAGAGNPPCLVDETAELVKAAQDIVSGASFDYNLPCIAEKSLIVVESVADRLLQQMQAFDALLITQPQEVDSLRKACLTPQGHANKNLVGKSPAELLKAAGITCPAKAPRLLLVEVAGDDPLVTTEQLMPLLPVVRVKDFDAALTLALQVEGGLHHTATMHSQNVSRLNLAARLLQTSIFVKNGPSYAGIGVGGEGFTTFTIATPTGEGTTSARTFARQRRCVLTNGFSIR

SEQ ID NO: 19 - protein, 460 aa, Escherichia fergusonii
  MISC_FEATURE (1)..(460): ATCC 35469
MNTRELENIIRNILREQLSTTADAPTNGIFDSVDEAINAAHQAFLRYQQCPLKTRSAIISAIRQELTPHLDMLATESANETGMGNKEDKFLKNKAALDNTPGIEDLTTTALTGDGGMVLFEYSPFGVIGSVTPSTNPTETIINNSISMLAAGNSVYFSPHPGAKNISLKLIAMIEEIAFRCSGIHNLIVTVAEPTFEATQQMMTHPNIAVLAITGGPGIVAMGMKSGKKVIGAGAGNPPCIVDETADLVKAAEDIINGASFDYNLPCIAEKSLIVVEEIAGTLVQQMQNFGALLLNKEETDKLRDVCLPQGMANKQLVGKSPAALLQAAGIAVPLKTPRLLIALVDACDKWVTSEQLMPMLPIVKVKDFDSALTLALKVEEGLHHTAIMHSQNVSRLNLAARTLQTSIFVKNGPSYAGIGVGGEGFTTFTIATPTGEGTTSAKTFARSRRCVLTSGFSIR

SEQ ID NO: 20 - protein, 464 aa, Salmonella enterica
  MISC_FEATURE (1)..(464): subsp. enterica serovar Urbana str. ATCC 9261
MNTSELETLIRTILSEQLTTPAQTPAQPKGKGIFQSVSEAIDAAHQAFLRYQQCPLKTRSAIISAMRQELTPLLATLAEESANETGMGNKEDKLLKNKAALDNTPGVEDLTTTALTGDGGMVLFEYSPFGVIGSVAPSTNPTETIINNSISMLAAGNSIYFSPHPGAKKVSLKLISLIEEIAFRCCGIRNLVVTVAEPTFEATQQMMAHPRIAVLAITGGPGIVAMGMKSGKKVIGAGAGNPPCIVDETADLVKAAEDIINGASFDYNLPCIAEKSLIVVESVAERLVQQMQTFGALLLSPADTDKLRAVCLPEGQANKKLVGKSPSAMLEAAGIAVPAKAPRLLIALVSADDPWVTSEQLMPMLPVVKVSDFDSALALALKVEEGLHHTAIMHSQNVSRLNLAARTLQTSIFVKNGPSYAGIGVGGEGFTTFTIATPTGEGTTSARTFARSRRCVLTNGFSIR

SEQ ID NO: 21 - DNA, 1434 bp, Lactobacillus reuteri
  MISC_FEATURE (1)..(1434): DSM 20016
ttaataccagttacgtactgagaatccttgtggtgagttcaaacgaacccgacgagtaaatgttcgtgcagtacatgttccttcaccagttggcgtagcaattgttaatgctgaagcacctgagtgagctccattatctgcaacacctgtaccaacataggatgggccattaacaacaaagattgaacattgcatccggtgagcagcattattaatatgcttaaggttattggaatgaatagtagctgtgtgatggttacctttttcaacttcaacagcagtcttcaaaacatcatcaaatgttggacaagaaacaactggtaaaattggcattaacatttcagtcattactaatggatgttccttaggaagttcacaaataatttcaactgggtggcctgtgtaaggaatattagcttggtctaagatataagttgcatccttaccaacaaatttacgatcaggagcaccattttcttggatacacatatcagctaatttatcggcttgttcacggttaactacaaaggcaccttcatcttgcatcttacgaattaattcatctttaatgctactttctgcaactacttccttttcagcagtacataaaatatcattatcaaatgaagcagatgtaatgatattatgagcagctaaatcaatatcagcagtagcatcaaccattgcaggaggattaccaggaccagcaccaaccgctttcttaccactggtcattgcttggtgaacaacagctgggccaccagttactgctaacattgcaatgtcggggtgcttcatcatttgttgaactgattcaattgttggtgtttcaatacttacaactaaattatgaaggcctgttgcatctgcaataaaatcgttcatcttttcaattgtccagcgagtaacattctttgcgccagggtgagcaccaaagtaaagagtattaccaccggcaagcatcatgatcgcattagcaattacagtttctgaagggtttgtacttgggccaaccgcaccaataacaccatatggtaaccgttcatacataaccatcccaccgtcaccgttttctacaactggttcaagaatctcgggaccaggagtgttgtacaaggcattgtttaacttagcaattttggcctctactgttcccattcctgtttcttctttgatatctttagccattttttcaatatatgggcggaatccttccttaatggcatcaatcacttgttggcgaacagcaattggcttatcccgatatatttcttgagcagcttttgcagcagcaattgcatcattgacattagtgaaaattccgcgatgaccattatcagtagtagctgcaacgtttgcacttgaagagctggcattatctagttcttcggcaagaattttgcgtacagcactttcaatatcattaatctgcat

SEQ ID NO: 22 - DNA, 1434 bp, Lactobacillus brevis
  MISC_FEATURE (1)..(1434): ATCC 367
ctaagcctcccaagtccgtaatgagaacccttctggcgagttaagccggcgccggcgcgtgtaagtcttagacgttgccgttccttcacccgttggtgtggcaatcgttaaggctgaagccccactatgcgcaccattcgttgcaacaccagtcccacaataagttgggccgttaaccacgaagattgacgtattcaaccggtgcgctgccttattgatgtgtggtaaattctcggaatgaatggaagccgtgtggtgtaacccaccttcaacttctgtagccgttgctaaaacgctatcaaagtcagggcaacaaacgactggcaaaattggcatcaacatttctgtcgttactaacggatgatccttagctgcttcaagaataatcagtgttggtgtcccggtgtaagagattcctgcttgatccaaaatgtaagtggcatctttaccaacaaacttccgatctggtgcgcccttaggcccaatggtcatttgcgctaatttttcaatatcggcagaatcggtaaccaagaaggccccttcttgttgcatccgcatgatgagttcatccttgacagcggcttcaacgacaacttccttttcggccgtgcagagaatgttattatcaaaggctgctgaatcaacaatgttgtgggctgctaaagcaatattggcagttgcatccaccattgccggtgggttaccagcaccggcaccaaccgccttcttaccactgataagcgcttggtggacaacagcaggccctccagtgattgacagcatggcaacgtcaggatgttgcataacttcttgcacggattcaattgaaggcgtttccagtgaaacgactaagttatgtaacccagttgcatcagctaccaattcgtttaatttttcgatcgtccaacgggtaatgttcttagcacctggatgggcaccaaagaacaacgtattcccaccagccaacatcatgatggcattggcaatcaccgtttcagaggggttggtactaggaccaacggcaccaatgacaccaaatggcgcgtattcatacataaccagtccaccgtcaccggtttcggcttctggctgcagaatttctggaccgggtgtgttataaagggcgttattgagtttggcaattttcgcgctaaccgttcccatgccagtttcgtcatgaatccgcttagccatatcctcaatgtatggccggaaaccctcacggatcgcatcaatcactttgttccgaactgagattggttggtcggcgtagttttcttgcgccgcttttgtggccgcaatggcttcattgaccgtcttaaagatcccatttttgccgggaacggtcgtattggtggccgttgatgactgaggattgctaagttcttcactcaaaattttacggatggcttgttcaatgttttctgtgttcat

SEQ ID NO: 23 - DNA, 1434 bp, Pediococcus acidilactici
ttatgcctcccacgaacgtaacgaaaatgcttctggcgaatttaaccgacgacggcgagtgaaggtcttagctgttgcggttccttctccagttggggttgcaatcgttaatgctgaagcacctgcatgagcaccgttagctcctaccccggttcccacatacgttgcaccgttaactacgaaaatggaagtgttcattcggtgtgccgcacggttaatatttggtaagttttcagaatgaattgaagccgtgtgatgcaatccttgttcaacttctaccgcagtcgctaaaacttgatcaaacgttgggcaagaaactactggtacaattggcatcaacatttcggtagtaaccaatggatgatccttttgggcttccaaaataattaattttggtgtgccagtgtatgcaattcctgccttatctaaaatgtaggttgcatctttaccgacgaattggcggtcaggtgcaccatttttgccaatggtcatttctgctaacttatcgatatcactagcgttagttaccaaaaatgctccttcttcttgcatctttttaataagttcatccttcacgctagattcaactactacttctttttctgcggtacaaaggatgttgttatcaaaagatgcggaatctacaatgttgtgtgcagctaacgcaacgttagcagtggcatcaacaattgcaggagggttacctgcaccagcacccacggcttttttgccactgactaatgcttggtgaaccactgctgggccgcccgtgatggacagcattgcaatgtcaggatgttgcatcatttcttgaacagattcaattgacggttcttcaattgatacaactaaattcttcatcccggtagcttcataaactaactcgtttaacttttcaattgtccaacgggtaatttttttagcacctggatgggcgccgaagtacaacgtatttccaccagctaacatcataatggcgttagcaattaccgtttcagaagggttagtacttggaccaactgccccaattactccaaacggagcgtattcgtacatcactaaaccaccgtcgccagtttcagcttcaggttgcaaaatttcagttccaggagtgttatataaagcattgtttagcttagcaattttagcttcaaccgttcccattcctgtttcatctttaatgtccttagccattttttcgatgtatggacggaatccttcacgaattgctgttaaaaccttttcccgaaaggctaatgttttatcaatgtaaacatcttcagctgccttagctgccgcgattgcttcatccacggtcttaaaaattccgttttggcccgcatcactagttgacgcagtttggctagtgccacttttctttagttcttcacttaaaatgcgtcgaatatcttcttcaagattttgaatttccat

SEQ ID NO: 24 - DNA, 1431 bp, Pediococcus claussenii
  MISC_FEATURE (1)..(1431): ATCC BAA-344
ttatgcctcccatgaacgaagtgagaatgcctcaggagaatttagacgacgacgtcttgtaaatgttttagcagttgctgtaccttcaccagtcggtgtggcaattgtcaaagctgatgctccagcatgcgcgccgtttgcacctactcctgtaccaacatacgttgcaccattaacaacaaaaattgatgtattcatacggtgtgctgcttgattaatatgtggtaagttctctgaatgaattgaagctgtatgatgtaaaccttgttcgacctcaacagctgttgctaatgcacggtcaaaggttgggcaggaaacaactggtaagattggcatcaacatttccgttgttactaatggatgattcttatgagcctcaagaataattagtttgggtgttccagtataattaattcctgcactatcaagaataaatgttgcgtctttaccaacaaatttacggtccggagcaccattcttaccaatcgtaatttgaactattttttcaatatcatgggcacttgtaaccaagaatgcaccctcgttttgcattttagaaattagttcatctttaacgctttcctcaacaacaacttctttttctgctgtacacaaaatattgttatcaaaagatgatgaatccacaatattatgtgcggctaaatcgatattagcagttgcatcaacaattgcaggtgggttaccagctccagcaccaaccgccttcttaccactaatcatagcttgatgaacaacaccagggcctccagtaattgaaagcatcgcaatatcaggatgttgcatcatctgttgaactgattcaattgaaggttcttcgattgaaacaaccatgtttctcataccagttgccttaaaaacaaattcatttaacttttcaattgtccatcgtgtgatttttttagcaccaggatgcgcaccaaagtacacagtgttaccaccagcaagcatcattaaagcatttgcaataactgtttcagatgggtttgtacttggtcctacagccccaatcactccaaatggtgcatattcatataatactaatccgccatcacccgtctcagcttctggttcgagtatttctgttccaggagtattgtatagagcattgttcaatttagcgatctttgcttcaacagtacccattcctgtttcttctttgatatctttagccatttgttcaatataaggccggaaaccttctttaatggcatctaatacttggtttcgaaaagccaatgatttgtcaacatatatttcttgcgcagctttagcggcagcaatcgcttcgtcaactgttttgaagattccgttcgttccattatcacttgaagcgcttacagaattatcactatcttgtagttcctcacttaaaattcggcggatatcttgttctaacttatccatttccat

SEQ ID NO: 25 - DNA, 1446 bp, Lactobacillus collinoides
atggcagatcaaaatattgaagcagaaatcagacgaattttacaagaagaattaagcggtaacgcttcgtccagcgctgctggtacgactaccagtcaacctgatgggttaggcaaccggatcttcaccaacgtgaacgatgccattgctgctgctaagcaagctcaggcaatctaccaagataaaccacttgccttccgtaaaaaagtcgttcaagcaattaaagatggtttcggcccatacattgaatatatggcaaagcagacccgtgaagaaactggcatgggaactgccgaagctaagattgctaagttaaagaacgccctctacaacaccccaggcgttgaattactggacccagaagttgaaactggtgacggcgggatggtcatgtatgaatacacgccattcggtgttatcggtgccgttggaccaagtacaaacccttgtgaaacggttctgaacaactccatcatgatgatgtctgctgggaacgcattgttctttggcgcccatcctggtgcaaagaacattactcgctgggcagttgaaaaattgaacgaattcgtttacaaggctactgggttgaagaacctcttagtttccttggacacaccatcaattgaatccgttcaagaaatgatgcaacatccagatgttgcaatgctggctgtaactggtggcccagctgttgtgcatcaagcattaacgagtggtaaaaaagccgttggtgccggtgctggtaacccgcctgcaatggttgatgcaactgctgatattgatttagcagctcataacctatttacttcagctaagtttgacaatgaaattctgtgtacttcagaaaaggaaatcattgctgaagattcaattaaggatgaacttcttcaaaagattgttgctaagggcgcttgcctagtaactgatcctaaagacatcaagcatttagctgacatgaccattggggacaacggtgcccctgaccggaaatatgttggtaaggatgccactgttatcttagatgccgctggtatttcatacaccggcgatcctaagttgatcatgatggatgttgataaagacaacccattggttaagacagaaatgttgatgccaatcttgcctatcgttgggtgcccagactttgacgccgttttggctacggctattgaagttgaaggtggcaatcaccatactgcttcaattcactcgaacaacatcctgcacatcaacaaggctgctcaccggatgaacacctcgatcttcgtcgcaaatggcccaacatttgccgcaactggtgtcggtgataacggttattacagtggtgctgctgcgctgacaattgctaccccaaccggtgaaggtactaccactactaagacctttacccgtcgtcgtcgtttcaactgtccacaagggttctcacttcgttcttgggaggtttaa

SEQ ID NO: 26 - DNA, 1410 bp, Listeria welshimeri
  MISC_FEATURE (1)..(1410): serovar 6b str. SLCC5334
atggaatcattagaactcgaacaactggtgaaaaaagttctgttagaaaaattagctgaacaaaaagatgtaccagtaaaaacaactacacaaggcgcaaaaagtgggatttttgatacagtggatgaggcagttcaagcagctgtccaagcacaaaatagttataaagaaaaatctctggaagaacgccgcaatgtagtaaaagcaattcgtgaagcactttatccagaaattgagtcaattgccacaagagcagttgctgaaacaggaatgggtaatgtgacagataaaattttgaaaaatactttagcgattgaaaaaacgccgggcgtagaagatttatatacagaagtagctactggtgataatggcatgacgctttatgaattatctccgtatggtgtaattggtgctgtggcgccgagtacgaatccaaccgaaacgttaatttgtaatacaatcggtatgcttgcagctgggaatgcagtgttttatagcccacatcctggtgcaaaaaatatatctctttggttgattgaaaagttgaatacgattgttcgtgaaagttgtggtattgataacttggttgtgacagtggaaaaaccttccattcaagcagcgcaagaaatgatgaatcatccaaaagtaccattacttgtcattacaggtggaccaggcgtcgtgcttcaagcaatgcaatcaggtaaaaaagtcattggagctggtgccggaaatccgccttccatcgtagacgaaacagctaatatcgaaaaagctgcagccgatattgttgacggagcctcttttgaccacaatatcttatgtattgctgaaaaaagcgttgttgccgttgatagcattactgatttcctattattccaaatggaaaaaaatggagcactacatgtgaccaatccgagcgatattaaaaaattagaaaaagttgctgtaacggataaaggtgtaacgaataaaaaattagtcggaaaaagcgcttctgaaattttaaaagaagctggaataacttgtgattttaccccgcgattaatcattgtggaaacagataaatcacatccatttgcaacagtagaattactaatgccaatcgttccagtggtaagagtgcctgattttgatgaagcgcttaaagtagctattgaattagaacaaggactacatcatacagcaacaatgcattcacaaaatatttccagattaaataaagctgcaagagatatgcaaacatcgatctttgtgaaaaatggtccttcctttgcaggtttaggttttagaggggaaggtagtactacatttactattgcaaccccaactggagaaggaaccactacagcacgtcattttgctagacgccgccgttgtgttttaacagatggtttttcgattcgttaa

SEQ ID NO: 27 - DNA, 1410 bp, Listeria innocua
  MISC_FEATURE (1)..(1410): Clip11262
atggaatcattagaactcgaacaactggtaaaaaaagttctcttagaaaaattagcagaacaaaaagaagtaccaacaaaaacaactacacaaggcgcgaaaagtggcgtttttgatacagttgacgaggctgttcaagcagcagttatagcgcagaattgctataaagaaaaatcacttgaagaacgccgcaatgttgtaaaagcaattcgtgaagcactttatccagaaattgaaacaattgcgacaagagcagttgcagagactggtatgggaaatgtgacagataaaattttgaaaaacacgttagcaatcgaaaaaacgccaggggtagaagatttatatacagaagtagctacaggtgataacggtatgacactatatgaactctctccgtatggcgtaattggtgcagtagcgccgagcacaaacccaacggaaacattgatttgtaattcaatcggtatgctcgcagctggaaatgccgttttttatagccctcatccaggggcaaaaaacatttcactgtggttgattgaaaaactaaacacaattgttcgcgatagttgtggtatagataatctaattgtcaccgtggctaaaccatccatccaagcagctcaagaaatgatgaaccatccaaaagtaccgctacttgttattacaggtggtccgggcgttgttctccaagcgatgcaatcaggtaaaaaagtgattggagcaggagcagggaacccgccttctattgttgacgaaacagctaatatcgaaaaagcggctgctgacatcgtagacggagcatcttttgaccataatattttatgtattgctgaaaaaagtgtggtagctgttgatagcattgctgatttcttgttattccaaatggaaaaaaatggtgcccttcatgttactaatccaagtgatattcaaaaattagaaaaagtagccgttaccgataaaggtgtaactaataaaaaattagtcggaaaaagtgcaactgaaatcttaaaagaagcaggaatagcttgtgattttacaccacgtttaatcattgtggaaacggagaaatctcatccatttgcaacagtagagctattaatgccaatcgttccagttgtaagggtgcctgattttgacgaagcccttgaagtggctattgaactcgaacaaggcttacatcatacagcaacaatgcattcacaaaatatctcgagattaaacaaagctgcaagagatatgcaaacttccatctttgtcaaaaatggtccgtcctttgcgggattaggctttagaggagaaggtagtactactttcactattgcaacgcctactggagaaggaacaactacagcacgtcattttgctagacgccgccgctgtgttttaacagatggtttttcgattcgttaa

SEQ ID NO: 28 - DNA, 1410 bp, Listeria monocytogenes
  MISC_FEATURE (1)..(1410): ATCC 19117
atggaatcattagaactcgaacaactggtaaaaaaagttcttttagaaaaattagcagaacaaaaagatgcaccagtaaaaacaacggtcaaaggcgcgaaaagtggggtttttgatacagttgacgaggccgttcaagcagcagttatagcacaaaataactataaagaaaaatcattagaagaacgccgcaacgttgtgaaagcaattcgcgaagcactttatccagaaattgaatccattgcagcgcgagcagttgctgaaacaggtatgggaaatgtagcagataaaattttgaaaaacacgttagcgattgaaaaaacgccaggtgtggaagatttgtatacagaagttgctactggtgataatggcatgacgctttacgaactttctccatatggcgtaatcggagctgttgcaccaagcacgaacccaacggaaaccttgatttgcaatacaatcggcatgctcgcagctgggaatgcagtattttatagcccgcatccaggtgcgaaaaatatttctctttggttgattgaaaagttgaatacgattgtccgtgaaagttgcggcattgataatttagttgttacagtcgaaaaaccatctattcaagccgcgcaagaaatgatgaatcatccgaaagtaccgctccttgttattacaggtggccctggtgtagttcttcaagccatgcaatccggtaaaaaagttattggcgcaggtgccgggaatccgccatctattgtagatgagacagcaaacatcgaaaaagcagctgctgatatcgtagacggcgcatcttttgaccataatattctatgtattgcggagaaaagtattgttgcagttgatagcatcgcagatttcttaatgttccaaatggaaaaaaatggtgcactacatgtgaccaatccaagcgatattcaaaaactagaaaaagtagctgtcacagataaaggcgtaacaaacaaaaaactagtcggaaaaagtgcttcagaaattttaaaagaagcggggattgtttgtgatttttcaccacgtttaattattgtggaaacagaaaaaacacatccgtttgcaactgtagaattattgatgccgattgttcctgttgtaagagttcctaattttgacgaagcgcttgatgtcgctattgagttagagcaaggcttgcatcacacagctacgatgcattcacaaaatatttctagattaaacaaagctgcacgagatatgcaaacatccatctttgtcaaaaatggtccttcatttgcgggattaggctttagaggagaaggtagcactactttcactattgcaacgcctaccggagaaggaaccactacagcgcgccattttgctagacgtcgccgttgtgttttaacagatggtttttcgattcgttaa

SEQ ID NO: 29 - DNA, 1410 bp, Listeria marthii
  MISC_FEATURE (1)..(1410): FSL S4-120
atggaatcattagaactcgaacaactggtgaaaaaagttcttttagaaaaattagcagaacaaaaagaagcaccagcaaaaccaataacacaaggtgcgaaaagtggtatttttgataccgtcgatgaagccgttcaagcagcagtaatagcgcaaaattgttataaagaaaaatcactagaagaacgccgcaatgttgtgaaagcaattcgcgaaactctttatccagaaattgaaacaatcgcgacgaaagcagtagcagaaacaggaatgggtaatgtagcagataaaattttgaaaaacactttagcgattgaaaaaactccaggggtagaagatttatatacagaagtagctactggcgataatggtatgacactttatgaactatctccgtatggcgttattggtgcagttgcgccgagcacgaatccgactgaaacattgatttgtaatacgatcggcatgctcgctgcgggaaatgcagtattttacagtccgcatccaggggcaaaaaatatttctctatggttgattgaaaaactaaatacaattgttcgcgaaagttgcggaattgataatttggtcgttacagtcgaaaaaccatctattcaagctgcacaagaaatgatgaatcatccgaaagtaccgttacttgtgattacaggtggcccaggcgtagttctgcaagcgatgcaatccggtaagaaagtgattggtgctggagccggaaatccgccgtcaatcgtagacgaaacagctaatattgaaaaagctgcggctgatatcgtggacggagcatcttttgaccataatattttatgtatcgcggaaaaaagtattgtggcagtagagagcattgctgatttcttattattccaaatggaaaaaaatggtgcactgcatgtgaccaatccaagtgatattcaaaaattagaaaaagtggcagtaacagataaaggcgtgaccaataaaaaattagttgggaaaagtgccgcagaaattttaaaagaagctggcataacttgtgactttaccccgcgtttaatcattgtagaaacgacaaaaacgcatccatttgcaacagtggaactattaatgccaatcgttccgcttgtaagagtgcctgattttgacgaagcacttgaagtagcaattgagttagagcaaggattacatcatactgcaacgatgcattcacaaaatatttccagattaaacaaagcggcaagagacatgcaaacatccatctttgtaaaaaatgggccttcatttgcaggattaggtttcagaggtgaaggtagcactacgtttaccattgcaacgcctaccggagaaggaaccactacagcacgtcattttgctagacgccgccgttgtgttttaactgatggtttttcgattcgttaa

SEQ ID NO: 30 - DNA, 1410 bp, Listeria ivanovii
  MISC_FEATURE (1)..(1410): subsp. ivanovii PAM 55
atggaatcattagaactcgaacaactggtgaaaaaagttctcttagaaaaattagcaggacaaaacgaagaaacaccaaaaaaaccaagccaaggtgccaaaagtggcatttttgacacagtggatgaggcagttcaagcagcagtaattgcgcaaaactgctacaaagaaaagtcgctagaagaccgcagaaatgtagtaaaagcaattcgcgaagcactttatccggaaatcgaaaatattgcgacacgtgcggctgctgaaacaggtatgggtaatgtagccgataaaattttgaaaaatacgttagcaattgaaaaaacaccaggagtagaagatctctatacagaagtagctactggcgataatggtatgacgctttatgaactttctccttatggtgttattggtgctgttgctccaagtacgaatccaacagaaacattaatttgcaacacaattggaatgcttgcagctggaaatgcagttttttatagcccgcatccaggtgcaaaaaatatttcgctttggttgattgaaaaactaaatacgattgttcgtgaaagctgcggaatcgataacctagtcgttacagtagaaaaaccatctattcaagcagcacaagaaatgatgaatcatccaaaagttccgttactagttatcactggcggccctggcgttgttcttcaagcgatgcaatccggtaagaaagtaatcggagcaggcgctggaaatccaccgtctatcgtagacgaaacagcgaatatcgaaaaagcagctgcagatatcgttgcgggcgcatcttttgatcataatattttatgtatcgcagaaaaaagcgtagtagcagtggacagcattactgattttctattattccaaatggaaaaaaatggcgcctttcatgttacgaatccaagcgatattcgcaaactggaaaaagtggcggttaccgaaaaaggcgttaccaacaagaagttagttggtaaaagcgcttcggaaattttaaaagaagcagggatagcatgtgattttacccctcgattaattattgctgaaacagatagatcccatccatttgcaacggtagaactgctaatgccaattgttccagttgtcagagtggctgattttgatcaagcacttgaagtagcacttgagttagaacaaggcttgcatcatacggcaacaatgcattcgcaaaatatttctagactgaacaaagcagcaagagatatgcaaacttctatttttgtgaaaaatggaccatcgtttgctggacttggctttggaggagaaggtagtgcgactttcactatcgctaccccaacaggtgaaggaactactacagcgcgacactttgctagacgccgtcgttgtgttttaacagatggtttttcgattcgttaa

SEQ ID NO: 31 - DNA, 1410 bp, Listeria seeligeri
  MISC_FEATURE (1)..(1410): serovar 1/2b str. SLCC3954
atggaatcattagaactcgaacaactggtgaaaaaagttctcttagaaaaattagcaggacaaaacgaagaaacaccaaaaaaaccaagccaaggtgccaaaagtggcattttcgatacagtggatgaggcagttcaagcagcagtaattgcgcaaaactgctacaaagagaagtcactagaagaccgcagaaatgttgtaaaagcaattcgtgaagcactttatccggaaatcaaaaatattgcgacacgtgcggttgctgaaacaggtatgggtaacgtagccgataaaattttgaaaaatacgttagcaattgaaaaaacaccaggagtagaagatctctatacagaagtagctacaggcgataatggtatgacgctttatgaactttctccttatggtgttattggtgctgttgctccaagtacgaatccaacagaaacattaatttgcaacacaattggaatgcttgcagctggaaatgcagttttttatagcccgcatccaggtgcaaaaaatatttcgctttggttgattgaaaaactaaatacgattgttcgcgaaagctgcgggattgataacctagtcgttacagttgaaaaaccatctattcaagcagcgcaagaaatgatgaatcatccaaaagtaccgttactagttatcactggcggtcctggtgttgttcttcaagcgatgcaatctggtaagaaagtaatcggagcaggtgcgggaaatccaccttctatcgtagacgaaacagcgaatatcgaaaaagcagctgctgatatcgttgcgggtgcatcttttgatcataatattttatgtatcgcagaaaaaagcgtagtagcagtggatagcatcactgattttctcttattccaaatggaaaaaaatggtgcgttgcatgttacgaatccaagcgatattcgcaaactggaaaaagtggcagttaccgaaaaaggcgttaccaataagaagttagttggtaaaagcgcttcggaaattttaaaagaagcagggatagcatgtgattttacccctcgattaattattgttgaaacagatagatcccatccatttgcaacggtagaacttttaatgccgattgttccagtggtacgagttgctgattttgatcaagcacttgaagtagcacttgagttagaacaaggcttacatcacacggcaacaatgcattcacaaaatatctctagactgaacaaagcagcacgagatatgcaaacatccattttcgtgaaaaatggaccatcgtttgctggacttggctttggaggagaaggtagtgcaactttcactatcgctaccccaacaggtgaaggaactactactgcgcgacactttgctagacgccgtcgttgtgttttaacagatggtttttcgattcgttaa

SEQ ID NO: 32 - DNA, 1395 bp, Shewanella putrefaciens
  MISC_FEATURE (1)..(1395): CN-32
ttagcgaatggaaaatccattggtcagcacacaacggcgtttacgggcgaagctccgtgctgatgttgtcccttcgccagtgggtgtcgcaatagtaaaggtggtaaaaccctcggcacctatgcccagtccggcataggaagggccatttttcacaaatattgaagtctgcatggtcttcgcagccaggttcagacgggtaacattctgagaatgcattatggcggtatggtgctgctcgttttccaccttcagagccagtgccagtcctgtctcgaaatcactgacccgtacaacaggcagaactggcattaattgttcgaccatcaccagcggatcgtcctgctccacttctacaataatcagtcgcggtgctgttgatgtgttcagatcagcagcctgcaggatcactgccggacttttacctaccagtttcttgtttgcttctcctttgtcattgataacgaccttgcgcagtctggcaatatcaccgggcgtttttaccagaaaggcatcgtttttctgcatattatccatgaggcgatcggcgatgctatctactacgatgacgcatttttcggcgatacaaaggacgttatgatcgaaagaggcaccatcaacgatatctttggcagccttgaccggacaggcagtctcatccaccagtacaggggggttaccagggccagcaccgataacttttttaccggttttcatggccatattgacaattgccggaccacctgtaaccgcaagcagagcaatgcgcggatctgacatcatttcgcgagtagcgtcaaaggtgggctctgctacggtggtaaccagattgcggatcccgcttacccgataaatgatgtcttcgattttttcaataagccacaaggataccttttttgcgcccggatgggggctgaagtagacagcattaccagcggccagcatactgatggtgttgttaatgatagtttcggtcggattggtgctgggggcgatagcgccaatgactccgaagggagatagttcgaataaaaccatgccaccatcaccagtcagggcgcttgtggtcaaatcctcaatccccggagtattattcagtgccgcagtgtttttactgattttgtcgggtgcattacccatgccggtttcttctgcggcgcgttcggacatctctttgatccagggtgccagctcttccttcagggctgtaatgatcctggttcgaagtgccagaggttctgccatgtattttttataggcatcgtagctggcagtgatagcatcttccacacgagcgaagatagtgtgctgaatgtttccaggggcagtagcggtccctttcagattatcagcaagaatattgcggatcatattctccagttcagtggtattcat

SEQ ID NO: 33 - DNA, 1395 bp, Kosakonia radicincitans
  MISC_FEATURE (1)..(1395): DSM 16656
ttagcggatggaaaatccattagtcagtacacaacggcgtttacgggcgaagctccgtgcagatgttgtcccctcgccagtgggagttgcaatagtaaaggtggtaaaaccctcagcctcaatgcccagcccagcataagaagggccatttttcacaaatattgaagtttgcatggtcttcgcggccaggttcagacgggaaacattctgcgaatgcattatggctgtatggtgctgatcgttttccactttcagcgccagtgccagccctgtctcaaaatccctgacccgcacaacaggcagaaccggcatcaattgttcgaccatcaccagcggatcgtcctgctccacttccacaataatcagtcgcggtgccgttgatgtgttcagatcagcagcctgcaggatcactgccgggcttttgcctaccagtttcttgtttgcttctcctttgtcattgataacgacctggcgcagtctggcaatatcaccgggtgtttttaccagaaaggcatcgtttttctgcatattttctacgaggcgatcggcgatgctatcgaccacgatgacgcatttttcggcgatgcaaaggacgttatgatcgaaagaggcaccatcaacgatatctttggcagctttgaccggacaggcagtttcatccaccagtacaggggggttacctgggccggcaccgataacttttttaccggttttcatggccatattgacaattgccgggccgcctgtaaccacaagcagagcaatgcgcggatctgacatcatttcgcgagtggcgtcaaaggtgggctctgcgacggtggtaaccagattccggatcccgcttacccgataaatgatgtcttcgattttttcaataagccacaaagataccttctttgcgccaggatgggggctgaagtagacagcattaccagcggccagcatactgatggtgttgttaatgatagtttcggtcggattggtgctgggggcgatagcgccaatgactccaaagggggaaagttcgaacaaaaccatgccaccatcgccagtcagggcgcttgtggtcaaatcctcaatccctggagtattattcagtgccgcagtatttttactgattttgtcgagtgcattacccatgccggtttcttctgcggcgcgttcggacatctctttgatccagggtgccagctcttccttcagggcagtaataatcctggttcgaagtgccagaggttctgccaagtattttttataggcatcgtagctggcagtgatagcatcttccacacgagcgaagatagtgtgctgaatatttccaggggcagtagcgatccctgtcagattatcagcaagaatagtgcggatcatattctccagttcagtggtattcat

SEQ ID NO: 34 - DNA, 1404 bp, Tolumonas auensis
  MISC_FEATURE (1)..(1404): DSM 9187
atgaataacactgagttagaaagcttaatccgcactattctgactgaacagctcacgccttccgctacggacacgcctgcatgtaccgcttcgtctgttgcactgtttgatgatgtggacagtgccatctgtgcagcgcatgccgccttcctgcgttatcaggaagcaccgttaaaaacccgcagtgccattattgccgccattcgtgctgagattgcgccctgcctgtctgaactggcagaacgtgctgccgcagaaaccggtatgggcaacaccgccgacaagatcctgaaaaacaaagcggcactggaaaatactcccggtatcgaagatttgaaaacaactgctctgaccggtgatgaaggtatggtgttgtttgaatactctccgtttggggtagttggtgccgtggcgccaagcacaaatccgaccgaaaccattatcaataacagcatcagtatgctggccgccggaaatgcgatctatttcagcccgcatcccggtgcaaaaaatatctctttgtggttaatccagaaaatggaagagatcgccttcaaagtctgcggtatccacaatctgatcgtgacggtcaaagagccgacttttgaagccacccagcaaatgatggcacatgacaaaatcgcgttgttagccatcaccggtggccccggtatcgtgaatatggggctgaaaagcgggaaaaaagtgattggtgccggcgccggtaatccgccttgtctggtggatgaaaccgcagagatcgtcaaagccgcacaagacatcgtcgcgggagcctcttttgactacaacctgccctgcatcgcagaaaaaagcgtgattgccgttgattgcatcgccgatcaactgattcagcaaatgcgcgaattcggcgccatgcagatcacggatcctcaacaaatcgcgcagttacgcgaagtctgcattcagaaaggtgcggctaataagagcctggtcggcaaaagcccggcaacgattctggcagccgcaggtattccctgcccggccaaagaaccgcgactgatcattctggaagtcccggccaatgacccgtttgttgttaccgaacaactgatgccggtgctgccgattgttcgcgttgataactttgaacaaggcctgcagctggcactgaaagtggaagatggcctgcaccatacggccatgatgcattcacagaatgtttcccgcctgaacaaggctgcacatctgatgcaaacttcaattttcgtgaaaaacggcccttcctacgcaggaattggtgtgggagcagaaggattcaccaccttcaccattgccaccccgaccggcgaaggcaccacatcagcccgcacgttcggtcgcttacgccgctgtgtactgaccaatggcttttcaattcgctaa

SEQ ID NO: 35 - DNA, 1386 bp, Citrobacter koseri
  MISC_FEATURE (1)..(1386): ATCC BAA-895
ttagcgaattgaaaagccgttggtcagtacgcagcgacgggaacgggcaaatgtgcgtgctgaggttgtcccttcgccggtcggggtggcgatggtaaaggtggtaaacccttcgccgccaacgccgataccggcataggaagggccgtttttcacaaaaatagaggtctgtaaggtgcgcgccgccagattcaggcgagagacattctgcgagtgcataatggcggtatggtgcaggccttcttcaactttcagcgccagtgccagcgcgctgtcgaaattatcgaccttcacgacgggtagcattggcatcaactgttcgctggttacccacggatcgtcggcactgacgataccgataagcaaacgcggcggttttgcgggaacggcaataccggcggcttccagcatggcggcagggcttttcccgaccagttttttattcgcgtgaccttccgggaggcagacggcgcgtaatttatcgatatccgcagcgtttaacagcaatgcgccgaaagcctgcatttgctgaaccaggcgctcagcgacgctctctacgacaatcagacttttttctgcgatacagggcaggttgtaatcgaacgctgcgccgttgatgatatcttcggccgctttgacgatatcggcggtctcatcaacgatgcagggcggattgcccgccccggcgccaatgacctttttaccgcttttcattcccatcgcaacaatgccggggccgccagtaatagccagcaccgcaatattgggatgcgccatcatttgctgagttgcctcaaaggtcggttctgcgacagtgacgaccagattacggatgccgcagcagcggaaggcgatatcttcgatcatgccgatcaatttgagtgagacgttcttcgcgccaggatgcgggctgaaataaacgctgttgcccgcagccagcatactgatgctgttgttaataatggtttcggtagggttggtgctgggcgcgacggaaccaatgacgccgaacggtgaatattcaaacagcaccatgccgccatcgccggtgagggccgtggttgtcaaatcctcaatgcctggcgtgttatccagcgcggctttgtttttaagaaatttatcttctttgtttcccatccccgtttccgctgcgctctccgccgccagcgtggcaagatgcggcgtaagctcctggcgcagggcgctgataatggcgctgcgcgttttgagcggacactgctgataacgtaagaaagcctggtgcgccgcgtctatcgcttcgccgacggactgaaaaataccgtgtccttgcgtttcggcctgcgcaggcgccaactgttcgcttaaaatattacggatgagggtttccagttcagaagtattcat

SEQ ID NO: 36 - DNA, 1389 bp, Yersinia enterocolitica
  MISC_FEATURE (1)..(1389): subsp. enterocolitica 8081
atgaataccaatgaccttgaatcgctcattcgcactatcctcaccgagcaactgacgccggtcacggcccctgcctccagcgccatttttgccagcgtggatgaagccattaatgctgctcacagcgcgtttttgcgctatcagcaaagcccgatgaaaactcgcagcgccattatccgcgctatccgtgagcaattaaagccacaacttgtctctctgtccgagcgcggtgccagtgaaaccggcatgggtaataaagaagataaattcctgaaaaacaaagctgcactggaaaacacaccgggtattgaagacttatctaccaccgccctgaccggtgatggcggcatggtgttattcgagtattcacccttcggcgttattggttcagtcacccccagcactaacccgaccgaaaccattattaataacagcatcagtatgttggcagcgggtaatgcagtctatttcagcccccaccctggtgctaaagccgtgtcactggatctcatcgcccaaattgaagagatcattttcaacagttgcggcattcgcaatctggtggtgacagtaaaagaaccgagtttcgaagccacccaacagatgatggcacacgacaaaattgccttactcgcgattactggtggcccggccattgtggcgatgagcatgaaaagcggcaagaaagtgattggtgccggtgcgggtaacccaccttgtctggtggatgaaaccgccgagttagtcaaagcggcgcaggatatcgtggcgggagcttcatttgactacaacctgccgtgcattgcagagaaaagcctgatcgtggtggaaagtgttgccgaccgtttattgcaacagatgcaggccttcgatgcattactgataagcaatccgcaagagatcgacagcttacgcaaagcctgcctgacgccgcagggccatgccaataaaaatctggtgggtaaaagtccaattgaactgctgaaagcagccggcatcacctgcccagctaaagccccgcgcctgttattggtcgaagtagctggtgacgatccactggtcaccaccgaacaattgatgccgctgttaccggtggtgcgggtaaaggattttgatgcggccctgacattggcactgcacgtcgagggcggcctgcatcataccgcaaccatgcactcacaaaatgtctcgcgcttgaatctggctgcacgtttgttgcaaacctccatttttgtcaaaaatggcccgtcctatgctgggataggggtcggcggtgaaggctttaccacctttactattgccaccccaaccggggagggtaccacttcggcgcgtacctttgcgcgtcaacgccgctgtgtactgactaatggtttctctattcgctga

SEQ ID NO: 37 - DNA, 1395 bp, Salmonella enterica
  MISC_FEATURE (1)..(1395): subsp. enterica serovar Mbandaka str. ATCC 51958
ttagcgaatagaaaagccgttggtcagtacgcagcgccgggagcgggcaaaagtacgcgctgacgtggtcccttcaccggttggcgtggcaatagtaaaggtggtaaagccttcgccgccgacgccgatcccggcataagaggggccgtttttgacgaatatcgaggtttgcagggtgcgggctgcgaggttcaggcgcgacacgttctgcgagtgcataatggcggtatgatgcagcccctcttcaaccttcagggccagcgccagcgcgctatcgaaatcgctgacttttaccaccggcagcatcggcatcagctgttcgctggtgacccacggatcgtcagcgctaaccagcgcaatcagcagacgcggcgcttttgcagggacagcgatcccggcggcttccagcatggccgatgggctcttgccgaccagttttttattcgcctggccttcaggcaggcagacggcgcggagtttgtcggtatcggcagggcttagcagcagcgcgccgaaggtttgcatttgctgcaccagacgctcggcgacgctctccactacgatcaggctcttctcggcaatgcagggcaggttataatcgaatgacgcgccgttgatgatatcttctgccgctttcaccaggtccgctgtttcatcgacgatgcagggcgggttacccgcgccagcgccaatcaccttcttaccgctcttcatgcccattgccacaatgcccgggccaccggtaatagccagtaccgcgattcgcgggtgggccatcatctgctgggtcgcttcgaaggtgggttcagctacagtcaccaccagattgcggatgccgcagcagcggaaggcaatctcttcaatcaggctaatcagcttcagagagacctttttcgctcccggatgcgggctaaagtagacgctgttgcccgccgccagcatgctgatgctgttgttgatgatggtttccgtcgggttggtgcttggggcgaccgaaccgatgacgccaaacggcgagtattcaaagagcaccatgccgccgtcgccggtcagcgcggtggtggtgagatcttctacgcccggcgtgttgtccagcgcagccttgtttttgaggagtttatcttctttgttgcccatccctgtttcattggcactctcttccgccagggtcgccagcagcggcgtcagctcctgacgcatcgcgctgataatggcgctgcgggtttttagcgggcactgctgataacgtaagaacgcctggtgcgcggcatcgatggcctcgctcacggactggaaaatccctttgccctgaggctgggccgtagtttgcgccggcgttgttaattgctcgctaagaatggtgcgaatcagggtttcgagttcagaagtattcat

SEQ ID NO: 38 - DNA, 1389 bp, Yersinia mollaretii
  MISC_FEATURE (1)..(1389): ATCC 43969
atgaacacccatgatattgaatctctcattcgcactatcctcaccgagcaactgacgcctgcgacggcctctgccgtcagcgccatttttgccagcgtggatgaagccgtgactgccgcccacagcgcctttttgcgctatcagcaaagcccgatgaaaacccgtagcgccattatcagcgccctgcgtgagcagttagcccctcagttggcgtcactctctgagcgtggtgccagcgaaaccggtatgggcaacaaagaagataaattcctgaaaaacagggccgcgctggagaatacccccggcatcgaagacctctccaccacggctctgacgggcgacggcggtatggtgctgttcgaatattcgccgttcggcgtgattggctctgtcgcccccagcactaaccccaccgaaaccattatcaataacagcatcagcatgttagccgcgggtaatgcggtctattttagcccgcaccccggcgctaaagccgtctcactggatctgattgcccaaattgaagcgatcattttcaaccgttgcggcatccgcaatttggtggtgacggtgcaagaaccgagctttgaggccacccaacagatgatggcccacgacaaaatcgctctactggcgatcaccggtgggccagccattgtggcgatgggcatgaagagcggcaaaaaagtgattggtgcgggcgcgggtaatccgccttgtctggtggatgagactgccgaactggtgaaagcggcgcaagatatcgtgtccggcgcgtcattcgactacaacctgccctgcattgccgagaagagtttgattgtggtggagagtgtcgccgaccgcctgttgcagcagatgcaagctttcgacgcgctgctgatcactcagccgcaagaggtcgatagcctacgcaaagcctgcctgaccccccaaggccacgctaacaaaaatctggtgggcaaaagcccggctgaactgctgaaagcggcgggtatcacttgccctgccaaagccccacgcctactgctggtggaagtggcgggtgacgatccgctagtgaccacggaacaactgatgccgctgctgccagtggtgcgggtaaaggattttgatgcggcgctgacactggcgctgcaagtggaaggcggcctgcatcacaccgcaaccatgcactcccagaatgtctcgcgcctgaatctggcggcccgcctattgcagacctccatttttgtcaaaaatggcccctcctatgcggggatcggggtcggcggcgagggctttaccaccttcaccatcgccacccccaccggagagggcaccacctcggcccgcacctttgcgcgtcaacgccgctgtgtgctgactaacggtttctccattcgctga

SEQ ID NO: 39 - DNA, 1383 bp, Escherichia fergusonii
  MISC_FEATURE (1)..(1383): ATCC 35469
atgaatacccgcgaactggaaaacatcatccgcaatattctgcgcgaacaactgagcacaacagcagatgccccgacgaatggcatttttgattctgttgatgaagcgattaatgccgcccatcaggcctttttgcgctatcaacaatgcccactgaaaacccgtagcgccattatcagcgccattcgccaggagctgactccacatctcgatatgttggcgacagaaagcgccaacgaaacaggcatgggcaataaagaggataaattcctcaaaaacaaagccgcgctcgataacacaccaggtattgaagacctgaccacaaccgcgctcactggtgatggcggcatggtgttatttgaatattcgccttttggtgttattggttctgtgacgccgagcactaacccaaccgaaaccattattaacaacagtattagcatgttagccgctggaaacagtgtctatttcagcccacatccgggggcaaaaaatatctctttgaaattgattgccatgattgaagagatcgcttttcgctgtagcggtatccacaacctgattgtcaccgttgctgaaccaacatttgaagccacacagcaaatgatgactcaccccaatatcgccgttctggcgattaccggtggacctggcattgtcgcaatgggcatgaaaagcggtaaaaaagtcattggggctggcgccggaaatccgccatgcatcgtagatgaaaccgcagatctggtaaaagctgcggaagatattattaatggtgcctcgtttgactacaacctgccctgcattgctgagaaaagcctgattgtcgttgaggagattgcaggtacgttggtgcaacaaatgcagaattttggcgctctgcttctcaacaaagaggaaaccgataagttacgtgacgtttgtctgccacaaggaatggcaaataaacaactggtaggtaaaagtccggcagctctgttgcaggcggcaggcattgctgtgccgctaaaaacaccacgtctgttaattgcccttgttgacgcctgcgacaagtgggtaaccagcgaacaacttatgccaatgctgccaatcgtaaaagttaaggatttcgatagcgcactgacgctggcactgaaagtggaagaaggtttgcatcacaccgccattatgcactcgcaaaatgtttcgcgactcaacctggcagcccggaccttacagacctcaatctttgttaagaatggtccgtcatatgctggtatcggtgtcggtggtgaaggatttaccacctttacgatcgctacccccacgggtgaaggtactacctcggccaaaacgtttgcccgttcccgtcgttgcgtgttgaccagcggtttttcgatccgttaa

SEQ ID NO: 40 - DNA, 1395 bp, Salmonella enterica
  MISC_FEATURE (1)..(1395): subsp. enterica serovar Urbana str. ATCC 9261
ttagcgaatagaaaagccgttggtcagcacgcagcgccgggagcgggcaaaagtacgcgctgacgtggtcccttcaccggttggcgtggcgatagtgaaggtggtaaagccttcgccgccgacgccgatcccggcataagaggggccgtttttgacgaatatcgaggtttgcagcgtgcgggccgcgaggttcaggcgcgacacgttctgcgagtgcataatggcggtatgatgcagcccctcttcaaccttcagggccagcgccagcgcgctatcgaaatcgctgacttttaccaccggcagcatcggcatcagctgttcgctggtgacccacggatcgtcagcgctaaccagcgcaatcagcagacgcggcgcttttgcagggacagcgatcccggcggcttccagcatggccgatgggctcttgccgaccagttttttattggcctgaccttcaggcaggcagacggcgcggagtttgtcggtatcggccgggcttagcagcagcgcgccgaaggtttgcatttgctgcaccagacgctcggcgacgctctccactacgatcaggctcttctcggcaatgcagggcaggttgtaatcgaatgacgcgccgttgatgatatcttccgccgctttcaccaggtctgctgtttcatcgacgatgcagggcgggttacccgcgccagcgccaatcaccttcttaccgctcttcatgcccattgccacaatgcccgggccaccggtaatggccagtaccgcgattcgcgggtgggccatcatctgctgggtcgcttcgaaggtgggttcagccacggtcaccaccagattgcggatgccgcagcagcggaaggcaatctcttcaattaggctaatcagcttcagagagacctttttcgcgcccggatgcgggctaaagtaaatactattgcccgccgccaacatgctgatgctgttattgatgatggtttccgtcgggttggtgcttggggcgaccgaaccgatgacgccaaacggtgagtattcaaacagcaccatgccgccgtcgccggtcagcgcggtggtggtgagatcttctacgcccggcgtgttgtccagcgcagccttgtttttgaggagtttatcttctttgttgcccatccctgtttcattggcactctcttccgccagggtcgccagcagcggcgtcagctcctgacgcatcgcgctgataatggcgctgcgggtttttagcgggcactgctgataacgtaagaacgcctggtgcgcggcatcgatggcctcgctcacggactggaaaatccctttgcccttaggctgggccggcgtttgcgctggcgtggttaattgctcgctaagaatggtgcgaatcagggtttcgagttcagaagtattcat

SEQ ID NO: 41 - protein, 299 aa, Dictyostelium discoideum
  MISC_FEATURE (1)..(299): Slime mold
MINRLFSINNIKNGSKFFSSSTTVETKQPLVLLEKHLVNGKYTGIQIVKLNKPKQLNALTFEMGVDYKKVVDTLAEDKDLKCVVLTGEGKAFSAGGDLDFLIERTKDTPENNQRIMERFYRTFLYIRSLPVPIISAINGAAIGAGFCLALATDIRVVSNKAPVGLTFTKLGIHPGMGVTHSITNIVGQDVASYMLLSSDIIKGDEAQRLGLVLKSVESDQVLPTALNLAETISKNSTIAVNSTTKTLRNKYNSDLDKSLTREADAQSQCWASKDIVEGILAIRESRDPKHNYLLFDDQK

SEQ ID NO: 42 - protein, 261 aa, Clostridium acetobutylicum
MELNNVILEKEGKVAVVTINRPKALNALNSDTLKEMDYVIGEIENDSEVLAVILTGAGEKSFVAGADISEMKEMNTIEGRKFGILGNKVFRRLELLEKPVIAAVNGFALGGGCEIAMSCDIRIASSNARFGQPEVGLGITPGFGGTQRLSRLVGMGMAKQLIFTAQNIKADEALRIGLVNKVVEPSELMNTAKEIANKIVSNAPVAVKLSKQAINRGMQCDIDTALAFESEAFGECFSTEDQKDAMTAFIEKRKIEGFKNR

SEQ ID NO: 43 - protein, 155 aa, Clostridium difficile
NSKKVVIAAVNGFALGGCELAMACDIRIASAKAKFGQPEVTLGITPGYGGTQRLTRLVGMAKAKELIFTGQVIKADEAEKIGLVNRVVEPDILIEEVEKLAKIIAKNAQLAVRYSKEAIQLGAQTDINTGIDIESNLFGLCFSTKDQKEGIVSFR

SEQ ID NO: 44 - protein, 258 aa, Clostridium pasteurianum
MGNIIFEEEDGIEKVTINRPKALNALNSETLKELGTVINDISVNDGIKAVIITGSGSKAFVAGADIAEMSTLNSIEATNFSRLAQNVFSQIENLPKLVVAAVNGFALGGGCELAMACDVRFASKKAKFGQPEVNLGILPSFGGTQRLPKLVGKGIAKELIFSTDMITADEAYRIGLANKVYEPEELLVKSQEFAEKVMTKSPWGVKLAKACINNGLDVDLEAGLKYEANSFGLCFSTEDQKEGMKAFLEKRKADFKGL

SEQ ID NO: 45 - protein, 262 aa, Clostridium pasteurianum
MDFNNIILEKEEKIAVVTINRPKALNALNSETLTELDSVIDEIDKDNEILAVVLTGAGKSFVAGADISEMKDMNVVEGRKFGILGNKVFRKLENLEKPVIAALNGFTLGGGCEIAMSCDIRIASTKAKFGQPEVQLGITPGFGGTQRLARLIGPGAAKELIYTGKIINAEEAYRLGLVNRVIEPETLLDEAKQLANTIAANAPIAVKLAKSAINRGIQTDIDTGVSIESEVFGACFSTEDQKEGMNTFLNDKKYLTGNFKNK

SEQ ID NO: 46 - protein, 260 aa, Megasphaera elsdenii
MDYQNIIFAVEDGIATITINRPKALNALNQATVSELKDVVEKIAADKAIKVVIITGAGAKSFVAGADIKEMASKNAAEGREWGQFGQNVFTEIENLPQPVIAAINGFALGGGCELSCACDIRYAAENAKFGQPEVGLGITPGFGGTQRLTRVVGRGHAKELIYTGGMIDAEKAKAIGLVNEVFPQEELMPAAVKLAKKIAKNAPIAVQLSKAAINRGINCDVVTGIAYEAEVFGLCFSTADQKEGMAAFCEKRKATFEGK

SEQ ID NO: 47 - protein, 259 aa, Metallosphaera sedula
MEFETIETKKEGNLFWITLNRPDKLNALNAKLLEELDRAVSQAESDPEIRVIIITGKGKAFCAGADITQFNQLTPAEAWKFSKKGREIMDKIEALSKPTIAMINGYALGGGLELALACDIRIAAEEAQLGLPEINLGIYPGYGGTQRLTRVIGKGRALEMMMTGDRIPGKDAEKYGLVNRVVPLANLEQETRKLAEKIAKKSPISLALIKEVVNRGLDSPLLSGLALESVGWGVVFSTEDKKEGVSAFLEKREPTFKGK

SEQ ID NO: 48 - protein, 259 aa, Clostridicum kluyvery
MEFKNIILEKDGNVASITLNRPKALNALNAATLKEIDAAINDIAEDDNVYAVIITGSGKAFVAGADIAEMKDLTAVEGRKFSVLGNKIFRKLENLEKPVIAAINGFALGGGCELSLSCDIRIASSKAKFGQPEVGLGITPGFGGTQRLARAIGVGMAKELIYTGKVINAEEALRIGLVNKVVEPDKLLEEAKALVDAIIVNAPIAVRMCKAAINQGLQCDIDTGVAYEAEVFGECFATEDRVEGMTAFVEKRDKAFKNK

SEQ ID NO: 49 - protein, 502 aa, Sulfolobus tokodaii
MAIRTGEQYLDSIKIRNKAEIYVMGKEVKDVTTHPFLKPSVMAFKATFDAAWEEDTKELARAWSPFINEEVNRFNHIHRSPEDLAAKVKLLRKLSHKTGACFQRCVGWDALNTLWIMTNIMAQKGKKEYKDRFVEYLSYVQKKDLALAGAMTDAKGVRTLKPHQQPNKNAYVRIEEVTKDGIYVSGAKANITGVAATEEIVVLPTRAMGPEDKDYAVAFSIPTDTEGIKIIVGRQLNDARRLEGGDIDALPYFYNHEGLVIFDHVFVPMDRVFLMGEYEFTSQLVEVFSAYHRQGYGGCKAGLGDVIIGASMNLAKQLGVEKASHVQEKLTEMIFLTETMYSAGIAASLNAVKVCDNCWWVNPMHANVTKHLVARFPAQISQLSIDIAGGIIGTAPSEWDLKNPKLREYIAKYLQGVEGYTAEDRLRMVRLLENVSLGVAFQIESVHGAGSPAAQRIMFSRLYDLNYAEEVAKRLAGKKTDLQWKPKAEPWRESETEKLVKS

SEQ ID NO: 50 - protein, 483 aa, Geobacter metallireducens
MALRDGNSYRESLRALNIKVYAFGEKIDSIVDHPLFQPHINAAALTFDLAHDPTTEALVTATSHLTGSKISRFTHIHQSTDDLIKKVKMLRLIAGKTGSCYQRCVGWDALNANYTVTYEMDQELGTDYHQRFRRYLEYIQDNDLMVAGAMTDPKGDRGLPPAKQKDPDMFVHVVAKNDKGIVIRGAKVHQTGIVNSHEMLIMPTMAMGEEDGDYAVACALPTDSPGVIHIFGRQTNDTRRLEKGDLDQGNAEYGTVGGEALTILEDVFVPWERVFMCGEYKYAGLLVERFASYHRQNYGGCKAGVSDVIIGATTAMAEYNGAAKASHVRDKIVEMVHLTETLYCGSIACSCEGAPTPSGAYFVNPLLANTVKQNVTRFIYEIARLSHDISGGCMATMPSEKDLHHDEIGKYVEKYFRGVDEAPTEERMRMARLVENMTGGTALVESMHGAGSPQAQRVMILRQANLGHKVKLAKKLAGIKEEK

SEQ ID NO: 51 - protein, 463 aa, Sulfolobus solfataricus
MRSKEDFLKSLKDGRNLYYRGKLVEDITTHQILKTAALHAAKLYEYADRVYEDNKMGKMSKFFKVPWTSQDLLDRHKLIYDLTMYCNGVFNISQAIGSDAIFALMITAKQVDRKYGTDYSKRVEKYFERVAKEDLTLATAQTDVKGDRSKRPSEQVDPDMYVRVVDVKSDGIVVRGAKAHTTQSAVSDEIIVIPTRVMRDSDKDYAVAFAVPANTKGLKMYIRPIDEIEGNSSSVLSRKDYELETLTVFNDVFVPWDRVFLFKEYDYAGTLAMLFATFHRFTALSYRSATMNLYLGASKVASQVNGIENEKHVRDDIVDIILYKEIMRSSAIAAAVYPVNMEGIAVPNPLFTNVGKLYSNMHFHDVVRDLIDIAGGIIATMPSQEDLESDEGKNIVKYLRGSVDGEERAKVLKLAKELGASTFTGYLLTGMIHAEGSMEASKIELFRSYNFKEAENLVKRVLS

SEQ ID NO: 52 - protein, 479 aa, Syntrophobacter fumaroxidans
MGLKTKAEYIESLRGMKPTVYMFGEKIESVVDNPRLRAGIEATGATYELAETEEYRPLIVTESPLIHEPVNRYTLPPSSIADLVARVKINRLMGTRVGTCFQRCTGLDCLSALSIVTYDIDAKHSTPYFKRFIEFLKHVQKNDLTCNAGVTDVKGDRSLAPHEQEDKDMYVRVVERNADGIVVRGAKAHQTGSLSSHEIIVLPTRALRKGDEDYALAFAIPNDTPGLIHVVGRSSLDTRQLDGCDLGNLHYSKYCPTVIFKDVFVPWERVFMCGEVEFAVEMVNRFSAYHRQSHGGCKSGKIDCMVGAALTMMDYNGTEKAGHLKQKAIEMVHRAETLYGCSLAASYEGKKEPSGTYFIDTVLANASKIHEGKEMSEAGRLLVDIAGGFVADLPSDRDLAIPEVGELLKKYLKGVASVPVEDRVKMYRLIEKLVMESADTISDIHGGGSPEAHRITILRESNLKAKKDAAKRLAGIESK

SEQ ID NO: 53 - protein, 486 aa, Porphyromonas gingivalis
MMTSEQYVESLRKLNLKVYFMGERIENPVDHPMIRPSMNSVAMTYKLAEMDEYKHLMTATSNLTGKQVNRFCHLHQSTEDLKDKVKMQRLMGQKTASCFQRCVGMDAFNAIYSTTYEMDQALGTTYHKRFIEYMKYVQDNDLVVDGAMTDPKGDRGLSPSEQADPDLYLHIVEVREDGIVVSGAKAHQTGAVNSHEHLIMPTIAMREADADYAVSFAVPSDAEGVIMIYGRQSCDTRKMEEGADIDLGNSEFGGHEALVVFDRVFVPNDRVFMCKEYQFAGMMVERFAGYHRQSYGGCKVGVGDVLIGAAALAADYNGVPKASHIKDKLIEMIHLNETLYACGIACSSEGTQMKAGNYMIDLLLANVCKQNITRLPYEIARLAEDIAGGLMVTMPSQQDFRHPEIGPIVKKYLAGATGKSTENRMRVLRLIENITLGTAAVGYRTESMHGAGSPQAQRIMIARQGDLEGKKKLARAIAHIDESLDK

SEQ ID NO: 54 - protein, 529 aa, Polynucleobacter necessarius
  MISC_FEATURE (1)..(529): subsp. Asymbioticus
MSQSTSQFMNSKDYQESLRSLKPTVYVDGRLIESVADEPSLRPGVQALGVTYDMVHDPALAPLMLADSNGTPVPRMLHINQSSGDLLNKLEAVRVLCQETGCAQRYLAHDALNAIAQVSARIDDAKGSNEHSAKFSEYLSHVQTKDLALGIAMTDAKGDRSRRPHEQENPDTYVHIVSQDAKGVVISGTKAIVTGAPYMHEFLVMPGRNMTKEDAAFAICCAVPVDAKGITIVARPAGRPGDKVEHGKPIFSSKYGQSTGVVIFDKVFVPWDRVFYAGEWEHSSVLTYNYATHHRHSCIAARAGFGDLLIGAGALMCEANGLDPATKSNLRDPMVELIKITEGFYACGVAASVYGTQDPYSKSFMPEPVFSNIGKLLLATQIYDMHRLAHEVSGGLIVALPGPDEDHNPATAATLAEVLRANPAVPYDKRIEVARFIEDLTASYQGGWYSVISLHGGGSPAAMKQEIYRQYPIGNKVELVERLLDRGVLTSSEERAITKNKQPGRCCDQGCSAPGQAVMVPLPEPGRRT

SEQ ID NO: 55 - protein, 257 aa, Sulfolobus tokodaii
METIVIKKETPIGWIYLNRPDRLNAINQQMIKELRQGIDEMVYDSDIKVIIITGNGKAFSAGADISRFKELNGYTAWQFAKSGRELMDYIENISKPTIAMVNGYALGGGLELAMACDIRIAAEEAQLGLPEINLGIYPGFGGTQRLVRLIGKGKALELMLTGDRISAKEAEKIGLVNKVVPLSNLEQETRNFALKLAEKPPISIALIKLLVNQGIDLPILAGLNMESLGWGVVFSTEDEKEGVSAFLEKRKAQFKGK

SEQ ID NO: 56 - protein, 258 aa, Gordonia terrae
  MISC_FEATURE (1)..(258): C-6
MTEHQTIVVETSGRVGIITLNRPKALNALNTELMNEVVGAVKEFDVDQGIGAIVITGSEKAFAAGADIKEMSSKSYADVVNEQFFGAWDELSRARTPIIAAVTGYALGGGCELAMLCDTIIAGDNAVFGQPEINLGVIPGIGGSQRLTRAVGKAKAMDMVLTGRQMKVDEAERLGLVSRVVPKEDCRAAAIEVAEIIASKSLIAAAAAKDAVNRAFESSLVEGVRAERALFYSTFATDDQTEGMAAFVEKRDPNFTHR

SEQ ID NO: 57 - protein, 258 aa, Halalkalicoccus jeotgali
MADRVLIERENDIATIIVNRPEKRNAMDIPTRKALYAAFEEVSEDDDVRAIVLRGAGDGSFIAGGDIDSFADFDHMDGMEYSEKYAQGLYNYVADRHKPTIAAVDGYALGGGTEIALACDIRLATDDAKFGLPEVGIGVIPAGGGTQRLVQVVGAGLASELILTGRIISADEAKRIGLANHVYAAEEFDNEVRAMAEDLASKAPVAQRLAKESIRRSLDIDAGLEYERLAGAFLFGTDDQKEGANAFLEDREPKYRNR

SEQ ID NO: 58 - protein, 257 aa, Carboxydothermus hydrogenoformans
MEFEKIKFEVTDGYAVIYLNNPPVNALGQKVLKDLQKALQEIEKNPEIRAVIISGEGSKVFCAGADITEFADRAKGILPEVEGSVLFRQIELFPKPVIAALNGSSYGGGTELAISCHLRILADDASMALPEVKLGIIPGWGGTQRLPRLIGKTRALEAMLTGEPITAEEALSYGLVNKVVPKDQVLTEARALAAKLAKGAPIAMREILKAVTLGLDTSIEEGLKIEKEGSKVAFSSEDAVEGRTAFFEKRPPNFKGR

SEQ ID NO: 59 - protein, 257 aa, Thermomicrobium roseum
MSVRVEREGAITLVTVERPERLNALDTATLRALLAAVQELATEEAIAVVVLTGAGDRAFIAGADISEMVEKSPAEALAFAELGHAVCRAIEEAPQPYIAAVNGYALGGGCEIALACDIRLASERAVFAQPEVTLGIPPGWGGSQRLPRVVPPGIARELLYTGRRVDAQEALRIGLVNAVYPADQLLERARELANRIAANGPLAVRLTKAAVRFGLEQGLEAGLTYERQVFAYAFTTEDQREGMRAFLEKRRPAFRGR

SEQ ID NO: 60 - protein, 274 aa, Methylobacterium extorquens
MNADAETASTDELLFAVDAAGIARITLNRPKARNALTFAMYRGLVELCERIEADHAIKAVIITGAGDKAFAAGTDIAQFRSFSKPEDAIGYERFMDRVLGGLERLRVPTIAAVAGACTGGGAAIAAACDMRIASRDARFGIPIARTLGNCLSQNTLRRLANLIGAPRVKDILFTARLVEAQEALAIGLVNEVVEDAAAVAARADALATLLASHAPLTLQATKEGLRRIGEEGAAEAAEGERPGDDLIVMTYMSADFREGMEAFLGKRPPNFKGR

SEQ ID NO: 61 - protein, 407 aa, Clostridium sporogenes
MSDRNKEVKEKKAKHYLREITAKHYKEALEAKERGEKVGWCASNFPQEIATTLGVKVVYPENHAAAVAARGNGQNMCEHAEAMGFSNDVCGYARVNLAVMDIGHSEDQPIPMPDFVLCCNNICNQMIKWYEHIAKTLDIPMILIDIPYNTENTVSQDRIKYIRAQFDDAIKQLEEITGKKWDENKFEEVMKISQESAKQWLRAASYAKYKPSPFSGFDLFNHMAVAVCARGTQEAADAFKMLADEYEENVKTGKSTYRGEEKQRILFEGIACWPYLRHKLTKLSEYGMNVTATVYAEAFGVIYENMDELMAAYNKVPNSISFENALKMRLNAVTSTNTEGAVIHINRSCKLWSGFLYELARRLEKETGIPVVSFDGDQADPRNFSEAQYDTRIQGLNEVMVAKKEAE

SEQ ID NO: 62 - protein, 374 aa, Clostridium sporogenes
MSNSDKFFNDFKDIVENPKKYIMKHMEQTGQKAIGCMPLYTPEELVLAAGMFPVGVWGSNTELSKAKTYFPAFICSILQTTLENALNGEYDMLSGMMITNYCDSLKCMGQNFKLTVENIEFIPVTVPQNRKMEAGKEFLKSQYKMNIEQLEKISGNKITDESLEKAIEIYDEHRKVMNDFSMLASKYPGIITPTKRNYVMKSAYYMDKKEHTEKVRQLMDEIKAIEPKPFEGKRVITTGIIADSEDLLKILEENNIAIVGDDIAHESRQYRTLTPEANTPMDRLAEQFANRECSTLYDPEKKRGQYIVEMAKERKADGIIFFMTKFCDPEEYDYPQMKKDFEEAGIPHVLIETDMQMKNYEQARTAIQAFSETL

SEQ ID NO: 63 - protein, 264 aa, Clostridium sporogenes
MADIYTMGVDIGSTASKTVVLKNGKEIVSQAVISVGAGTSGPKRAIDSVLKDAKLSIEDLDYIVSTGYGRNSFDFANKQISELSCHAKGVYFDNNKARTVIDIGGQDIKVLKLADSGRLLNFIMNDKCAAGTGRFLDVMSRVIEVPVDELGKKALESKNPCTISSTCTVFAESEVISQLARGVKTEDLIAGICKSVASRVASLAKRSGIEELVVMSGGVAKNIGVVKAMEAELGRDIYISKNSQLNGALGASLYAYESFQKERS

SEQ ID NO: 64 - protein, 412 aa, Clostridium sporogenes
MENNTNMFSGVKVIELANFIAAPAAGRFFADGGAEVIKIESPAGDPLRYTAPSEGRPLSQEENTTYDLENANKKAIVLNLKSEKGKKILHEMLAEADILLTNWRTKALVKQGLDYETLKEKYPKLVFAQITGYGEKGPDKDLPGFDYTAFFARGGVSGTLYEKGTVPPNVVPGLGDHQAGMFLAAGMAGALYKAKTTGQGDKVTVSLMHSAMYGLGIMIQAAQYKDHGLVYPINRNETPNPFIVSYKSKDDYFVQVCMPPYDVFYDRFMTALGREDLVGDERYNKIENLKDGRAKEVYSIIEQQMVTKTKDEWDNIFRDADIPFAIAQTWEDLLEDEQAWANDYLYKMKYPTGNERALVRLPVFFKEAGLPEYNQSPQIAENTVEVLKEMGYTEQEIEELEKDKDIMVRKEK

SEQ ID NO: 65 - protein, 368 aa, Lachnoanaerobaculum saburreum
MWHCLETLKKISASPKEQLNKYLEEGKKVIAVAPVYTPEEIIHAFGFVPMGVWGADIEINESKKYYPAFICSIMQTVLELGIKGNYNGVSAIVVPSLCDSLKTLGQNWKYAVKDIPFIPMTYPQNRKSDYAVDFTLEMYKRVISDLENITGEKFDEGKLKNTYEIYNEHNRVMREFTKVSEEYEVSATDRSAVFKSAWFMLKEEHTELVRELIELIKKEGKISKKLRIYTTGILADAPDLLNIFDSNNMQIVGDDIAYESRQYRTDIPDGNGLYALAKKFSNMDNCTLLYDKDKRRVDFIIEEAKKKRADGIVVLMTKFCDPEEFDYVPIKRAANEAGIPHINIEVDRQMKNYQQANTMLQTFADMLV

SEQ ID NO: 66 - protein, 409 aa, Lachnoanaerobaculum saburreum
MEEAKKQKPTVDPNSAKARLGRIAAKAYSDCVEAKKRGELVGWCASNFPVEIPETLGLYVCYPENQAAGIAARGGGERMCSESEGDGYSNDICAYARISLAYMKLKEAPEQDMPQPDFVLCCNNICNCMIKWYENIAKELNIPMIMIDIPFNPDYEVSDAMTAYIRNQFWDAIHQLEEITGKKWSNERYEEVRKISGRSSRAWLEATATAKYSPSPFNGFDLLNHMAVMVTARGKLEAAEAMETLLQEYKDNHEKGESTFKGEEKYRIMFEGIACWPWLRATATGLKSRGINMVTTIYADAFGFIYDDFDGMCRAYANVPNCMNIEHARDKRIKLCKDNSVEGLLVHTNRSCKLWSGFMSEMSRQIGEECGIPVVSFDGDQADPRNFSEAQYDTRVQGLTEIMEANKEI

SEQ ID NO: 67 - protein, 256 aa, Lachnoanaerobaculum saburreum
MYTLGVDIGSTTSKAVILEDGENIVASSIVIATVGTAGVEEAVKNVLNFSKLELNDIKAVVATGYGRMNYDVADYKVSELTCHALGVHKEFPNVRTVIDIGGQDAKVISLAANGKMTNFVMNDKCAAGTGRFLDVMANILNLDIQDLEVEALKSDNPANISSTCTVFAESEVISQLATGRNIPDLVAGICKSVAVRVAALAKRVGIVEEVCMSGGVAKNSGVRNAMSKELGVDIVFSKDAQLMGALGAAIYGFKKL

SEQ ID NO: 68 - protein, 264 aa, Peptostreptococcus stomatis
MSSVYTMGIDIGSTSSKCVIMKDGKEIVSEGVVSLGAGTKGSDLVIEEVLGKAGMTFDEIDLIVSTGYGRNSYERAAKTVSELSCHAKGGGYIFGGAGTIIDIGGQDIKVLKLNDKGGLVNFLMNDKCAAGTGRFLEVMSGVLDVKLDELGELDAKATEVTPISSTCTVFAESEVISCMAKKIPLENIIRGIHASVATRVASLARRGGLKTPVAMTGGVSKNKGIVRALKEELECDILISPDSQMAGAIGAALYAYDEYQKQNA

SEQ ID NO: 69 - protein, 372 aa, Peptostreptococcus stomatis
MSNIDVLLGKLDVSLLGQVDKYVSEGKKVIGCAPVYTPEELVYAAGMVPIGVWGAEGEVGLSKEYFPAFYAAIILRLMDLGLEGKLDKMSGMIIPGLSDGLKGLSQNWKRAIKQVPALYIGYGQNRKIEAGITYNEKQYIKLRGQLEEIAGCKIEDAKVEEAIVLYNKHRKAMQEFSSLAASHLNTITPILRARVMTSAFLFDKAEHLAILEELNKELKALPEEKFAGKKVVTTGILANSPGMLEILDEYKLGIVDDNINHESGQFDYLVDEGTGNPVRALSKWISDIEGSTLLYDPEKLRGQIIIDKVKKHQADGVIYLMTKFSDSDEFDYPIIRKELENAGILHILVEVDQQMTNFEQAKTALQTFADMI

SEQ ID NO: 70 - protein, 411 aa, Peptostreptococcus stomatis
MSNTGMVEEKPAKVLLGEIVAKHYKEAWEAKNNGELVGWCASNFPQEIFETMDIKVVYPENQAAAISAKGGGQRMCEIAENEGYSNDICAYARISLAYMDVKDAPELNMPQPDFVACCNNICNCMIKWYENIAKELNIPLILIDVPYNNDYEAEDDRVEYLRGQFDYAIKQLEELTGKKWDEKKFEEVMEVSQRTGRAWLKATGYAKYTPSPFSGFDVFNHMAVAVCARGKIESAIAFEKLAEEFDENVRTGKSTFKGEEKFRVLFEGIACWPHLRHTFKQLKDAGVNVCGTVYADAFGYIYDNTYQLMQAYCGTPNAISYERATDMRLKVIEENNIDGMLIHINRSCKQWSGIMYEMERDIREKTGIPTATFDGDQADPRNFSEAQYDTRVQGLIELMEANKAAKMKEAH

SEQ ID NO: 71 - protein, 408 aa, Clostridium difficile
MSEKKEARVVINDLLAEQYANAFKAKEEGRPVGWSTSVFPQELAEVFDLNVLYPENQAAGVAAKKGSLELCEIAESKGYSIDLCAYARTNFGLLENGGCEALDMPAPDFLLCCNNICNQVIKWYENISRELDIPLIMIDTTFNNEDEVTQSRIDYIKAQFEEAIKQLEIISGKKFDPKKFEEVMKISAENGRLWKYSMSLPADSSPSPMNGFDLFTYMAVIVCARGKKETTEAFKLLIEELEDNMKTGKSSFRGEEKYRIMMEGIPCWPYIGYKMKTLAKFGVNMTGSVYPHAWALQYEVNDLDGMAVAYSTMFNNVNLDRMTKYRVDSLVEGKCDGAFYHMNRSCKLMSLIQYEMQRRAAEETGLPYAGFDGDQADPRAFTNAQFETRIQGLVEVMEERKKLNRGEI

SEQ ID NO: 72 - protein, 375 aa, Clostridium difficile
MEAILSKMKEVVENPNAAVKKYKSETGKKAIGCFPVYCPEEIIHAAGMLPVGIWGGQTELDLAKQYFPAFACSIMQSCLEYGLKGAYDELSGVIIPGMCDTLICLGQNWKSAVPHIKYISLVHPQNRKLEAGVKYLISEYKGVKRELEEICGYEIEEAKIHESIEVYNEHRKTMRDFVEVAYKHSNTIKPSIRSLVIKSGFFMRKEEHTELVKDLIAKLNAMPEEVCSGKKVLLTGILADSKDILDILEDNNISVVADDLAQETRQFRTDVPAGDDALERLARQWSNIEGCSLAYDPKKKRGSLIVDEVKKKDIDGVIFCMMKFCDPEEYDYPLVRKDIEDSGIPTLYVEIDQQTQNNEQARTRIQTFAEMMSLA

SEQ ID NO: 73 - protein, 266 aa, Clostridium difficile
MYTMGLDIGSTASKGVILKNGEDIVASETISSGTGTTGPSRVLEKLYGKTGLAREDIKKVVVTGYGRMNYSDADKQISELSCHARGVNFIIPETRTIIDIGGQDAKVLKLDNNGRLLNFLMNDKCAAGTGRFLDVMAKIIEVDVSELGSISMNSQNEVSISSTCTVFAESEVISHLSENAKIEDIVAGIHTSVAKRVSSLVKRIGVQRNVVMVGGVARNSGIVRAMAREINTEIIVPDIPQLTGALGAALYAFDEAKESQKEVKNI

SEQ ID NO: 74 - protein, 399 aa, Clostridium difficile
MLLEGVKVVELSSFIAAPCCAKMLGDWGAEVIKIEPIEGDGIRVMGGTFKSPASDDENPMFELENGNKKGVSINVKSKEGVEILHKLLSEADIFVTNVRVQALEKMGIAYDQIKDKYPGLIFSQILGYGEKGPLKDKPGFDYTAYFARGGVSQSVMEKGTSPANTAAGFGDHYAGLALAAGSLAALHKKAQTGKGERVTVSLFHTAIYGMGTMITTAQYGNEMPLSRENPNSPLMTTYKCKDGRWIQLALIQYNKWLGKFCKVINREYILEDDRYNNIDSMVNHVEDLVKIVGEAMLEKTLDEWSALLEEADLPFEKIQSCEDLLDDEQAWANDFLFKKTYDSGNTGVLVNTPVMFRNEGIKEYTPAPKVGQHTVEVLKSLGYDEEKINNFKDSKVVRY

SEQ ID NO: 75 - protein, 255 aa, Escherichia coli
  MISC_FEATURE (1)..(255): strain K12
MSELIVSRQQRVLLLTLNRPAARNALNNALLTQLVNELEAAAIDTSISVCVITGNARFFAAGADLNEMAEKDLAATLNDTRPQLWARLQAFNKPLIAAVNGYALGAGCELALLCDVVVAGENARFGLPEITLGIMPGAGGTQRLIRSVGKSLASKMVLSGESITARQAQQAGLVSDVFPSDLTLEYALQLASKMARHSPLALQAAKQALRQSQEVALQAGLAQERQLFTLLAATEDRHEGISAFLQKRSPDFKGR

SEQ ID NO: 76 - protein, 257 aa, Rhodobacter capsulatus
MSYHTIRYEISEGLAVITLDRPEVMNALNAAMRHELTAALHRARGEARAIVLTGSGRAFCSGQDLGDGAAEGLNLETVLREEYEPLLQAIYSCPLPVLAAVNGAAAGAGANLALAADVVIAAQSAAFMQAFTRIGLMPDAGGTWWLPRQVGMARAMGMALFAEKIGAEEAARMGLIWEAVPDVDFEHHWRARAAHLARGPSAAFAAVKKAFHAGLSNPLPAQLALEARLQGELGQSADFREGVQAFLEKRPPHFTGR

SEQ ID NO: 77 - protein, 701 aa, Pseudomonas stutzeri
MTDVIRLERRGDIALILVNNPPVNALGHAVRKGLLDAFQEADEAPEVTAVVLVCEGPTFMAGADIKEFGKPPQAPSLPEVIEVIEGCRKPSVAVIHGTALGGGLEVALGCHYRIARSDAKVGLPEVKLGLLPGAGGTQRLPRLAGVEKALEMIVSGQPIGAAEALEHYIVDELFEGDLIEAGLTYARRLVEEGRGPRRSGEQTRGLEGVDNEALIRAKHAEVAKRMPGLFSPLRCIAAVEAATRLPLAEGLKRERELFTECLNSPQRGALIHSFFAERQAGKIDDLPSDVTPRPIRTAAVIGGGTMGVGIALSFANAGVPVKLLEINDEALQRGLQRARETYAASVKRGSLTEDAMEQRLALIAGVTDYGALADADVVVEAVFEEMGVKQQVFEQLDAVCKPGAILASNTSSLDLNAIAGFTRRPEDVVGMHFFSPANVMRLLEVVRGERTSDEVLAAAMAIGKQLKKVSVVVGVCDGFVGNRMVFQYGREAEFLLEEGATPQQVDAALRNFGMAMGPFAMRDLSGLDIGQAIRKRQRATLPAHLDFPTVSDKLCAAGMLGQKTGAGYYRYEPGNRTPQENPDLAPMLEAASREKGIERQALDEQYIVERCIFALVNEGAKILEEGIAQRSSDIDVIYLNGYGFPAFRGGPMYYADSVGLDKVLARVKELHARCGDWWKPAPLLEKLAAEGRTFTEWQAGQ

SEQ ID NO: 78 - protein, 655 aa, Haliangium ochraceum
MIVGVIGSGAIGPDLAYGFASALASVPGARVYLHDIKQEALDAGMQRIRGYIAKGLARGKISERVAGALETVLVPTLSLADLAPCSYVLEAATEELGVKRAILRSLEDTVDSECLIGFATSGLPRAIIAAEVKHPERCFVNHPFYPAWRSLPVEVVLSGSPAHGQRMLATLEALGKVPVITADAPCFAADDIFCNYCSEAARIVEEGIANPAQVDAIVHGAIGGGGPLNVLDATRGNLLTVHCQELMRDADTGTPWFEPPAILRERGDALWHDPKAPHDPAFDEALRERVLDRILAVLLARTVFVLDHGICAATELDWMTRTALGFRTGLVDLVDELGPERVAELCQRYAAEHPGFVIPDSIREQHKPRFYGNLRVTRQDELAIVRIFRPEVKNALDRRTLSELDHLMAALSADDSVEGVVLSSAGGALAGADITELARVRTTEEAVSTCAFGQAVLNRIAAMDKPVVAAVDGPVLGGGAELSMACHARVVGPRLSMGQPEVNLGIIPGYGGTQRLPRLIGVERALAMMRTAQSIDAQTACEWGWASGTPMVDFVGAAATLIRSHLAGEAELAPLDPAPMSVPAAAAPVDIGHRSRVIDEILVDVVQSGLRAPLSEGLATEAAGFGRCVLTVDLDIGLKNFMQNGPRVPALFLHE

SEQ ID NO: 79 - protein, 255 aa, Anoxybacillus flavithermus
MFSIQQEGYVAILALHRPPANALASSVLKELSERLDALKEDEQVRVIVLHGEGRFFSAGADIKEFTAIEASEQAAELARAGQQVMEKIEQFPKPIIAAIHGAALGGGLELAMSCHLRIVAENAKLGLPELQLGIIPGFAGTQRLLRHVGMAKALEMMWTSEPITGAEAVQWGLANKAVPEEQLLDTAKQLAQKIAQKSPISVQAVLKLVNEARTKTFHECVEKEAQLFGQVFVTEDAKEGISAFIEKRTPQFQGK

SEQ ID NO: 80 - protein, 260 aa, Streptomyces avermitilis
MSTAPEAADLVLHERHGGVLTITINRPAQKNAVDHEAAVQLAAAVDLLDADPELSVGVLTGAGGVFSAGMDLKAFAKGELPLLPSRGLGGLTRASVRKPLVAAVEGWALGGGFELVLACDLIVAAEDARFGFPEVMRGLVAAEGGLVRLPRRLPYHVAARVLLTGEPLTAVEAKEYGLVNELTPPGAALDAARELAGRVARNAPLALAAVKEVLRETQGLKESDAFRRQDELTSGLAASEDAREGAQAFAEKRAPVWHGR

SEQ ID NO: 81 - protein, 560 aa, Advenella kashmirensis
MDNGRKLIERGWHLFNRIEKLAFPTLALMHGPCLGGGLELALACRYRIAIDSPKPVIGLPEVKLGIFPAWGGLMRLPRLIGPQTALNMMLTGRTLDGRKARSAGLVDLLVAPRVAEKSAIDLVTSGKPARQARGLAGLLNRAPFKSLVAAQARKSVKQKDPYGHYPATLTMLDLWEKHDGDPLADPQALTRLLQSDVTRNLIRVFHLQERLKAFGKKDNATPVNHVHVIGAGVMGGGIAAWCALQGIKTTLQDTDAQRIAGAFKNAVSIYARKDRYTAQAARDRLIPDLAGHGIATADLVIEAISENPQAKQSLYQQIEPKMKEGAILATNTSSLSIAQLRSVLVHPERFVGIHFFNPVSRMPLVEVVHADGIAQETLDTAAAFVGKIGKLPLPVQDTPGFLVNAVLAPYMLQAMRCIDEGMDPEVIDTAMLEFGMPMGPITLADTVGLDIAMAAGKQLSEGQEPPRCLQEKIAQGKLGVKSGEGFYVWKDRKHDQRSSKAIPQGLAQRLIKPLIEQTEKQLANNIVQDADLADAGVIFGTGFAPFTGGPIHYKQSKGGL

SEQ ID NO: 82 - protein, 237 aa, Oligotropha carboxidovorans
MSLSPLANGVRVLTLDRPSKANALNAEVVDQLLACVAQAEAEDCRVLILAANGKAFCGGFDFGGYESMSAGDLLLRFVRIEELLQRMRQSSFVSIALVHGAAMGAGADIVASCTYRIGTDASRFRFPGFRFGVALGTRHLAQLVGPQRARDILLTNATIDALTAVDIGLLTHLVDAGSMRQKADEIIAQIGSLDRVARNRILHLTSAQNNDGDMAELVKSVSAPGLHERIAQYRAGH

SEQ ID NO: 83 - protein, 266 aa, Riemerella anatipestifer
MYKLIDVDNHFEGKLQIAYINQPESFNSLNKVVLEELLHFIKACDADSSVRCIAISGKGKAFCSGQNLKEALDYKAEANEERFIQRIVIDYYNPLVKAIVYAKKPVIALVNGPAVGAGAMLALICDFAVASESAYFSLAFSNIGLVPDTAGTYYLPKLLGRSLASYLAFTGKKLSAKESLERGLVVDVFSDATFSEQSLQVLEHITHQPTVALGLTKKAFNKSYQNSLSEQLDLESILQQDAAETWDFQEGIAAFLAKRKPQYKGK

SEQ ID NO: 84 - protein, 422 aa, Fusobacterium necrophorum
  MISC_FEATURE (1)..(422): subsp. funduliforme Fnf 1007
MSETINLDEMSAKQLLGYYQEKLDEEARQAKREGKLVCWSASVAPPEFCVAMDIAMVYPETHAAGIGARKGSLDLLEVADEKGYSLDICSYARVNLGYMELLKQQALTGETPEKLANSPAAKVPLPDLVITCNNICNTLLKWYENLAKELNIPCIVIDVPFNHTMPITKHSKEYIADQFKYAIQQLEEITGKKFDYDKFLEVQEQTQRSVYQWNRLAALAHYKPSPLNGFDLFNFMALIVCARSRDYAEITFKKFADELEENLKNEVYAFKGAEKNRVTWEGIAVWPYLGHTFKSLKGMGSIMTGSAYPGIWNLTYTPGDMESMAEAYTRVYINTCLQNKADVLSKIVTDGKCDGILYHLNRSCKLMSFLNVETAELVEKATGVPYVSFDGDQTDPRNFAPAQFDTRVQALNEMMEVNNETK

SEQ ID NO: 85 - protein, 277 aa, Fusobacterium necrophorum
  MISC_FEATURE (1)..(277): subsp. funduliforme Fnf 1007
MQDDRSFKKGKRRGMYTVGVDIGSSSSKVVILKDGTEIVSQSAIQSGIGSNRAIVALEDNLKKANLTKEDIGFTVVTGYGRFTFEGADKQISEISCHARGIHFLLPNVRTIIDIGGQDAKAISLDEKGHVRQFFMNDKCAAGTGRFLTVMARVLEISLDEMGTYDALSKNPCNISSTCAVFAESEVISQLAKGNTKEDVIAGVHNSVAHKILGLVYRTSMEEKFAICGGVAQNTGALRAIREALKKEVIVAPNPQLTGALGAAIFAYDELKKLRKGE

SEQ ID NO: 86 - protein, 374 aa, Fusobacterium necrophorum
  MISC_FEATURE (1)..(374): subsp. funduliforme Fnf 1007
MKGRLEELIHIFEDVANNPKKMVAEYKKEVGKEVIGVMPVYAPEEIIHAAGCLPIGLWGGKKEVSKARAYLPPFACSIMQTVMELQIGGTYDILDAVLFSVPCDTLKCLSQKWKGKSPVIVFTHPQNRVIEGANAYLVKEYQAVKEKLEGILGRTIPMEAIEESVKVYNENRRVMREFVEVAAQYPQIIDPIVRHNVMKSRWFLRKEKHTEYVKELIAELKKETIVPWDGKKVILTGIMTEPVELLQIFKDEKLAIVADDLAHESRQFRGDVPEEGGDVLYRMAKWWQNLEGCSLATDTNKGRGQMLMDMCKDTKADAVIVCMMKFCDPEEFDYPVYYREFTESGIKNITVEVDLEVSSFEQIRTRIQTFKDIL

SEQ ID NO: 87 - protein, 422 aa, Desulfosporosinus youngiae
  MISC_FEATURE (1)..(422): DSM 17734
MTDTTTMSAKELLGFYQEELYEEARQAKKEGKLVCWSASVAPSEFCVAMDVAMIYPETHAAGIGARKGALDVLEVADEKGYNLDTCSYARVNMGYMELLKQEALTGITPEKLEKSPAARIPLPDFVITCNNICNTLLKWYENLAVELNIPCIIIDVPFNHTMPIPQYAKDYIAEQFKEAITQLEEICGRKFDYDKFLKVQEQTQRSVAQWNRIAALSGHKPSPLNGFDLFNYMALIVCARSRDYAEITFKKFADELEENLKNGIYAFKGNEQKRVTWEGIAVWPHLGHTFKGLKNLGNIMTGSAYPGLWNLTYTPGDMSSMAEAYTRIYINTCLDNKVKVLSDVISGGKCDGVIYHQNRSCKLMSLLNVETADILQKQNHLPYVSFDGDQTDPRNFAPAQFDTRIQALDEMMKQNKEGVSNE

SEQ ID NO: 88 - protein, 372 aa, Desulfosporosinus youngiae
  MISC_FEATURE (1)..(372): DSM 17734
MSRIETIISELTSIANNPRQAMEDYKKETGKGSVGVMPYYAPEEIIHAAGYLPVGIWGGQKSISKARAYLPPFACSIMQSVVEMQLEGVYDDLEAVLFPVPCDTLKCLSQKWKGTSPVIVLTHPQNRKLEAANKFLAEEYRLVREKLEKILNVKITDEALNQSIEIYNENRKVMREFTEIAANYPNIIDPVKRHALIKARFFMEKAKHTALVKELNAELKALPVEAFTGKKVVLTGIMAEPNEVLDILQDNGFAVVADDLAQESRLFRNDVPSGTDPLYRLAKWWQEFDGCSLAVDAKKPRGPMLMDMVKASKADAVVVCMMKFCDPEEFDYPIYYRQFEEAGIKSLFIEIDLEPTSFEQTKTRVQSFREML

SEQ ID NO: 89 - protein, 272 aa, Desulfosporosinus youngiae
  MISC_FEATURE (1)..(272): DSM 17734
MFTMGIDIGSSSSKVVILEDGVNIIAGEVIQIGTGSTGPKRVLDEALAKAGLTLQDMAKIIATGYGRSSVEEAHKQISEISCQAKGVFFLVPSAKLIIDIGGQDVKAIKLDSKGCVKQFFMNDKCAAGTGRFLDVMSRVLEVNLDEMAEYDARATEPATVSSTCTVFAESEVISQLANGVAKENIIAGVHQSVASKACGLAYRCGVEEDIVMCGGVAKDLGVVRAISKELKKPVIVAPNPQITAALGAAIFAFEEVMETVMVAFEEVRGANK

SEQ ID NO: 90 - protein, 422 aa, Peptoniphilus indolicus
  MISC_FEATURE (1)..(422): ATCC 29427
MNTIDISNMKAKEMLGYFQNKLDEEAREAKKNGKLVCWSASVAPSEFCVTMDIALVYPETHAAGIGARKGSLAMLDVADRKGYNTDICSYARVNLGYMELLKEYAKTGVKPKELEESPAADVPLPDLVITCNNICNTLLKWYENLAAELNIPCIVIDVPFNHTMPIPKYSKEYIADQFKEAIRQLEEITGKDFDYDKFLEVQEQTQRSVAQWNRLAALSKYEPSPLNGFDLFNYMALIVCARSKNYAELTFKKFADELEENMQNGVYPYKAGEQSRITWEGIAIWPYLGHTFKTLKGYGSIMTGSAYPGLWNLEYTPGDMLSMAEAYTRIYINTCLDNKVDVLRKIIKNGKCDGVAYHLNRSCKLMSLLNVETAEILNKENNLPYVSFDGDQTDPRNFSEAQYDNRIQTLTEMMSANKKMRG

SEQ ID NO: 91 - protein, 263 aa, Peptoniphilus indolicus
  MISC_FEATURE (1)..(263): ATCC 29427
MYTMGVDIGSTSSKIIILEDGIKIIGNIVVQSGTGTSGPTIATAKAKSFLSNNNLTLDDISKIVVTGYGRFSFDIADKQISEITCHTKGINFLVPEARTILDIGGQDTKAISVNDKGQVLQFFMNDKCAAGTGRFLEVMAKILEIPLEKMGEYDRLSTNPVAISSTCTVFAESEVISQLSKGISKENILAGVHNSTANKVCGLLYRTGIKEKIVLCGGVAQNQGVVRALQEELKKEITIAPHPQMTGAIGAALFAYEEANKNL

SEQ ID NO: 92 - protein, 372 aa, Peptoniphilus indolicus
  MISC_FEATURE (1)..(372): ATCC 29427
MNKINEIINLLDEVSKDPKLTVKKYKEKTGKGVVGVMPLYAPEEIIHAAGFLPMGLWGAQKEVSKARIYLPPFACSIMQTNMELQIEGAYDDLDAVVFSVPCDTLKCMSQKWKGKSPVIVFTHPQNRKLESANKFLVTEYEILKDKLEKILNVKISDESITNSIEIYNENRKVMREFSDLAGQYPNIIDPIQRHIVFKSRWFMEKSEHTKLVKELISEIKKLPIEEWDGYKVIATGIMIEPEEILQIFKDKKIAIVADDLAQESRQFRHDVPEGDQPLLRLAKWWQNLEGCALATDTKKLRGQMLIDMAKKYNADAVLICMMKFCDPEEFDYPVYYREFQEAGIKNLLIEIDLEMTAFEQTNTRLQTLVETL

SEQ ID NO: 93 - protein, 422 aa, Desulfosporosinus meridiei
  MISC_FEATURE (1)..(422): strain ATCC BAA-275 / DSM 13257 / NCIMB 13706 / S10
MTDTTAMSAKELLGFYQEELYEEARRAKKEGKLVCWSASVAPSEFCVAMDVAMIYPETHAAGIGARKGALDVLEVADEKGYNVDTCSYARVNLGYMELLKQEALTGITPEKLEKSPAARIPLPDFVITCNNICNTLLKWYENLAVELNIPCIIIDVPFNHTMPIPQYAKDYIAEQFKEAITQLEEICGKKFDYDKFLKVQEQTQRSVAQWNRIAALSSHKPSPLNGFDLFNYMALIVCARSKDYAEITFKKFADELEENLNKGIFAFKGNEQKRVTWEGIAVWPHLGHTFKGLKNLGNIMTGSAYPGLWNVSYTPGDMSSMAEAYTRIYINTCLDNKVKVLSDVISGGKCDGVIYHQNRSCKLMSFLNVETADILQKENGLPYVSFDGDQTDPRNFSPAQFDTRIQALDEMMKQNKEGVSNE

SEQ ID NO: 94 - protein, 372 aa, Desulfosporosinus meridiei
  MISC_FEATURE (1)..(372): strain ATCC BAA-275 / DSM 13257 / NCIMB 13706 / S10
MSRIETIISELSSISNNPRKAMEDYKKETGKGSVGVMPYYAPEEIIHAAGFLPVGIWGGQKSISKARAYLPPFACSIMQSVMEMQLEGVYDDLEAVLFPVPCDTLKCLSQKWKGTSPVIVFTHPQNRKLEAANKFLAEEYRLVREKLETILNVKITDEALNQSIETYNENRKVMREFTDLAANYPQIIDPRIRHAIIKARFFMEKSKHTAMVKELNSELKSLPVEAFTGKKVVLTGIMAEPNEVLDILKDNGFAVVADDLAQESRLFRNDVPSGTDPLYRLAKWWQEFDGCSLATDAKKSRGPMLMEMVKGSKADAVVVCMMKFCDPEEFDYPIYYRQFEEAGIKSLFIEIDLETTSFEQTKTRVQSFSEML

SEQ ID NO: 95 - protein, 261 aa, Desulfosporosinus meridiei
  MISC_FEATURE (1)..(261): strain ATCC BAA-275 / DSM 13257 / NCIMB 13706 / S10
MFTMGIDIGSSSSKVVILEDGVNIIAGEVIQIGTGSTGPKRVLNEALSKAGLKLEDMAKIIATGYGRSSVEEAHKQISEISCQAKGVFFLVPSAKLIIDIGGQDVKAIRLDSKGGVKQFFMNDKCAAGTGRFLDVMSRVLEVNLDEMAEYDARATEPATVSSTCTVFAESEVISQLSNGVAKENIIAGVHQSVASKACGLAYRCGVEEDIVMCGGVAKDLGVVRAISKELKKPVIVAPNPQITAALGAAIFAFEEVRGANK

SEQ ID NO: 96 - protein, 477 aa, Acidaminococcus fermentans
MPKTVSPGVQALRDVVEKVYRELREAKERGEKVGWSSSKFPCELAESFGLHVGYPENQAAGIAANRDGEVMCQAAEDIGYDNDICGYARISLAYAAGFRGANKMDKDGNYVINPHSGKQMKDANGKKVFDADGKPVIDPKTLKPFATTDNIYEIAALPEGEEKTRRQNALHKYRQMTMPMPDFVLCCNNICNCMTKWYEDIARRHNIPLIMIDVPYNEFDHVNEANVKYIRSQLDTAIRQMEEITGKKFDEDKFEQCCQNANRTAKAWLKVCDYLQYKPAPFNGFDLFNHMADVVTARGRVEAAEAFELLAKELEQHVKEGTTTAPFKEQHRIMFEGIPCWPKLPNLFKPLKANGLNITGVVYAPAFGFVYNNLDELVKAYCKAPNSVSIEQGVAWREGLIRDNKVDGVLVHYNRSCKPWSGYMPEMQRRFTKDMGIPTAGFDGDQADPRNFNAAQYETRVQGLVEAMEANDEKKGK

SEQ ID NO: 97 - protein, 379 aa, Acidaminococcus fermentans
MAISALIEEFQKVSASPKTMLAKYKAQGKKAIGCLPYYVPEELVYAAGMVPMGVWGCNGKQEVRSKEYCASFYCTIAQQSLEMLLDGTLDGLDGIITPVLCDTLRPMSQNFKVAMKDKMPVIFLAHPQVRQNAAGKQFTYDAYSEVKGHLEEICGHEITNDAILDAIKVYNKSRAARREFCKLANEHPDLIPASVRATVLRAAYFMLKDEYTEKLEELNKELAAAPAGKFDGHKVVVSGIIYNMPGILKAMDDNKLAIAADDCAYESRSFAVDAPEDLDNGLQALAVQFSKQKNDVLLYDPEFAKNTRSEHVCNLVKESGAEGLIVFMMQFCDPEEMEYPDLKKALDAHHIPHVKIGVDQMTRDFGQAQTALEAFAESL

SEQ ID NO: 98 - protein, 260 aa, Acidaminococcus fermentans
MSIYTLGIDVGSTASKCIILKDGKEIVAKSLVAVGTGTSGPARSISEVLENAHMKKEDMAFTLATGYGRNSLEGIADKQMSELSCHAMGASFIWPNVHTVIDIGGQDVKVIHVENGTMTNFQMNDKCAAGTGRFLDVMANILEVKVSDLAELGAKSTKRVAISSTCTVFAESEVISQLSKGTDKIDIIAGIHRSVASRVIGLANRVGIVKDVVMTGGVAQNYGVRGALEEGLGVEIKTSPLAQYNGALGAALYAYKKAAK

SEQ ID NO: 99 - protein, 336 aa, Carboxydothermus hydrogenoformans
MKLNYFCSYWPVEISEGAGISTVRYFPSDESKAPVRLPAYCCSYARGSLAEIEEEGDGDFWGFAHSCDTMQSLYGITKSLLGDDRVFLFVPPVDLTTAFAREYYREALIYLWRELSQKSGVNGEEKLKLTWEKLKELRNKVKSLENLTSIIPSSEIFELLKKLQTLPLDEALDYLEAKKAEFTSLSVAQKAIGIILTGAVVTNSKLYLALEQQGFRVVYDDTCTGFRHFAGEIEDKDDILEAIVSYYLSKPPCPCRHKGVWARAEYLKNLYHNKNARAIVLLQNKFCDPFAWDVPYLVDYFKKQGVPVLVLEVEGGEIGEQNKTRLQAFRESVGGV

SEQ ID NO: 100 - protein, 404 aa, Carboxydothermus hydrogenoformans
MAKKIFKPLKASEKINKILKNHYLKAKYLPTLGKFFGYKTAWITSGAPVELLRAFGIEPVYPENYGAICGARKVSPSLCQVAENRGYSLDLCSYAKSNLGSIWNPKESPFNGLPRPDLLVVCNNICGTVLKWYETLSREFNIPLFIIDTPFITGEPQPWQIQYVAKQIEKLAIELEKFFRKKLDLNRLEKVILLANETVDLWKGIRNFAKNKPSPVNVTDLFINLGPMVVLRGTEVARDFYEEVYREVEERYKAGVPAVEGEKYRLVWDNIPIWYGLYRFYGYFAERGAVFVTDSYTGGWAVNIKKGPPFYALAETYAGVFLNRDLEFRKNQLQSFIEEFSADGFVMHSNRSCKAYSFVQEEIRRQIMRSLGVPGLIVDADMTDSRLYSEETVLNRVQAFLESL

SEQ ID NO: 101 - protein, 254 aa, Carboxydothermus hydrogenoformans
MYLGVDIGSLTTKVVLIDRGKNLIAYRYSKTGPAGKETAERLIQEVLIKANISRDDIQGIVATGYGRVLFSGKEFSEITCQARGIGHLYPEAKTIIDIGGQDSKVISLGKNGKVLDFAMNDKCAAGTGRFLEVMSQALEVRLEEIGELAEKSQEAAKISSVCTVFAESEVISNLSRGQSREAVARGICEAVAARTAILAQKVGVVEPVVFTGGVAKNTGVVAALERKLGVKLLIPEDSTITAALGAALLAAENS

SEQ ID NO: 102 - protein, 261 aa, Oscillibacter valericigenes
MNNIYTMGIDVGSTASKCLILKDGSEIVAKSLVDVGAGTSGPTRAIAEVLEAAGMKKEDMAFILATGYGRNSLDDIADHQMSELSCHAKGAFFLFPDVHTVIDIGGQDVKILEIENGVMVNFAMNDKCAAGTGRFLDVMARVLEVKVEDLADLGAQSTKNVEISSTCTVFAESEVISQLAKGSDKRDIIHGIHKSVASRVVGLANRIGVRDAVVMTGGVAQNGGVVSALQEALGHPIHTSPLTQYNGALGAALFAWQKATK

SEQ ID NO: 103 - protein, 427 aa, Oscillibacter valericigenes
MAENEKATAAAPEAAPVKKAPKPVSPGTQALRDVVTKVYAAAWDAKKAGRPVGWSSSKFPCEIAEALGLAVVYPENQAAGIGAQHDGQRMCESAESLGFDPDICGYARISLAYSAGVETTNESRRVPMPDFVLCCNNICNCMTKWYENIARMHNIPLIMIDVPYNNEVTVSDSQVAYIRGQFDDAIKQMEKIAGVKFDEKKFEQACANANRTAKAWLTVCDYLQYKPAPMSGFDLFNHMADVVTARGKVETAEAFELLASELEQHVKNGTSTAPFPEQYRVMFEGIPCWPNLRTLFKPLKANGVNVTAVVYAPAFGFVYNGLDEMARAYCKAPNSVCIEQGVDWREGICRENKVDGVLVHYNRSCKPWSGYMAEMQRRFTKDLGVPCAGFDGDQADPRNFNEAQYETRVQGLVEAMEENKKQKEARA

SEQ ID NO: 104 - protein, 380 aa, Oscillibacter valericigenes
MSIETIVKEFADVAADPKAQLKKYKAEGKKCIGVMPYYAPEELVAAAGMVPFGMWGSNDKTISRAKEYCATFYCTIAQLDLEMLLDGTMDLLDGVITPTICDTLRPMSQNIRVAMGEKLPCIFLAHPQNRKPAYGKKFCLDQYTHIKTELEKIAGAPITDAALSETIKVYNKSRAARREFVKLVSDHCDVITPTKRSAVLKAAWFMPKAEYTEKLKALNAELKALPVCDWKGTKVVTSGIICDNPKLLEIFEENKIAIAADDVAHESRSFRVDAPETGDPMEALAQQFANQDYDVLLYDEHSSENRRGEFVAKLVKDSGAKGLVLFMQQFCDPEEMEYPSLKKALDEAKIPHIKLGVDQQMRDFGQARTAIQAFADVISL

SEQ ID NO: 105 - protein, 422 aa, Desulfosporosinus orientis
  MISC_FEATURE (1)..(422): strain ATCC 19365 / DSM 765 / NCIMB 8382 / VKM B-1628
MTDTANMSAKELLGFYQEELYEEARQAKKEGKLVCWSASVAPSEFCVAMDVAMIYPETHAAGIGARKGALDMLEVADEKGYNLDTCSYARVNLGYMELLKQEALTGITPEKLEKSPAARVPLPDFVITCNNICNTLLKWYENLAVELNIPCIVIDVPFNHTMPIPQYAKDYIAEQFKEAIAQLEEICGKKFDYDKFLQVQEQTQRSVAQWNRIASLSGHKPSPLNGFDLFNYMALIVCARSRDCAEITFKKFADELEDNLSKGIYAFKGNEQKRITWEGIAVWPHLGHTFKGLKNLGNIMTGSAYPGLWNLSYTPGDMSSMAEAYTRIYINTCLDNKVKVLSDIISGGKCDGVIYHQNRSCKLMSFLNVETADILQQQNHLPYVSFDGDQTDPRNFAPAQFDTRIQALDEMMKQNKEGVSHE

SEQ ID NO: 106 - protein, 372 aa, Desulfosporosinus orientis
  MISC_FEATURE (1)..(372): strain ATCC 19365 / DSM 765 / NCIMB 8382 / VKM B-1628
MSRIEAIISELSSIANNPRKAMEDYKKETGKGSVGIMPYYAPEEIVHAAGYLPVGIWGGQKSISKARAYLPPFACSIMQSVVEMQLEGVYNDLAAVLFPVPCDTLKCLSQKWKGTSPVIVMTHPQNRKLEAANKFLAEEYRLVREKLEKILNVQITDEALNHSIDVYNENRKAMREFTDIAANYLNIIDPRKRHEIIKARFFMEKSKHTALVKELNSELKSLPVEDFTGKKVILTGIMAEPNEVLDILKENDFAVVADDLAQESRLFRIDVPAGPDPLYRLAKWWQEFDGCSLAVDTKKLRGPMLMNMVNVDKADAVVVCMMKFCDPEEFDYPIYYRQFEEAGIKSLFIEIDLEPTSFEQTKTRVQSFREML

SEQ ID NO: 107 - protein, 266 aa, Desulfosporosinus orientis
  MISC_FEATURE (1)..(266): strain ATCC 19365 / DSM 765 / NCIMB 8382 / VKM B-1628
MYTMGIDIGSSSSKVVILEDGVNLIAGEVIQIGTGSTGPKRVLEEALAKTGLTLADMAKIIATGYGRSSVEVSDKQISEISCQAKGVYFLVPTAKLIIDIGGQDVKAIRLDRIGGVRQFFMNDKCAAGTGRFLDVMSRVLEVDLDEMAEYDARATEPATVSSTCTVFAESEVISQLANGVAKENIIAGVHQSVASKACGLAYRCGVEEDVVMCGGVAKDLGVVRAISKELKKPVIVAPNPQITAALGAALFAYEEVMEANKLRKEV

SEQ ID NO: 108 - protein, 411 aa, Peptostreptococcus anaerobius [sequence omitted]

SEQ ID NO: 109 - protein, 372 aa, Peptostreptococcus anaerobius
  MISC_FEATURE (1)..(372): CAG621
MSNLEELFGKLAVCPLEQIDKYVADGKKVIGCAPVYAPEELVYASGMIPMAIWGAEGEVTLAKEYFPAFYVSIILRLLDLGLEGKLDKMSGMILPGLSDGLKGLSQNWKRAVKNVPALYIGYGQNRKIEAGIVYNARQYEKLKVQLEEIAGKKIEDAQIEEAIVLYNKHRKAMQAFSDLAAKHLNTVTPSLRAKVMSSACLMDKAEHLEIVEAINAELSAMPEEKFDGKKIVTTGLLANSPEILKIFEEFKLGIVADNINHESGQFDYLVDEATGNPIKALSKWISDIEGSTLLYDPEKLRGQIIIDKAKKYDADGVVYLLSKFSDSDEFDYPIIRKQLEEAGYMHILVEVDQQMTNFEQAKTALQTFADMI

SEQ ID NO: 110 - protein, 263 aa, Peptostreptococcus anaerobius [sequence omitted]

SEQ ID NO: 111 - protein, 258 aa, Chloroflexus aggregans
  MISC_FEATURE (1)..(258): strain MD-66 / DSM 9485
MSDETLVLSTIEGPVAILTLNRPQALNALSPALIDALIRHLEHCDNDDTIRVIIITGAGRAFAAGADIKAMADATPIDMLTTDMIARWARIAAVRKPVIAAVNGFALGGGCELAMMCDIILASETAQFGQPEINIGIIPGAGGTQRLTRAIGPYRAMEMVLTGATISAQEAYAYGLVNRVCPPDSLLDEARRLAQTIAAKPPLAVRLAKEAVRAAAETTVREGLAIELRNFYLLFASADQKEGMRAFIEKRTANFSGR

SEQ ID NO: 112 - protein, 257 aa, Marivirga tractuosa
MEFIKVNTQYKKHIALINLNRPKELNALNLQLMTELKDTLKVLDEDENVRVIILTGNEKAFAAGADIKQMAGKTAIDMLNVDQFSTWDQIKKTKKPLIAAVSGFALGGGCELAMTCDMIVASESAKFGQPEIKIGVMPGAGGTQRLTRAIGKAKAMELVLTGNFISAEEAMHYGLVNKVVPTEMYLEAAAELAEQIAQMSPVAAKLAKESVNRAFETHLDEGLHFERKNFYLTFASEDQTEGMEAFVEKRKPEFKGK

SEQ ID NO: 113 - protein, 257 aa, Marinithermus hydrothermalis
  MISC_FEATURE (1)..(257): strain DSM 14884/JCM 11576/T1
MYENLIVETLEGGVGLIRIHRPKRLNALNQATMDEIVRAVRAFEADDAVRAIVLTGDERAFAAGADVTEMDGANVPEMLSGYRFEQWETLRRTTKPLIAAVSGFALGGGLELAMLCDIIVASETARLGQPEINLGIMPGAGGTQRLTRQVGKYLAMEMVLTGRMLTAEEAYRHGLVNRVVPVEFYLEEAIQIAREIAKKAPVAVRLAKDAILKAEDTPLEVGLAYERHNFYLLFGTEDKQEGIRAFLEKRKPEWKGR

SEQ ID NO: 114 - protein, 259 aa, Chitinophaga pinensis
  MISC_FEATURE (1)..(259): strain ATCC 43595/DSM 2588/NCIB11800/UQM 2034
MQPQFIIIHRQVAPYVAHIQLNRPKELNALNLELMIELRDALKMLDADDNVRAIVISGNEKAFAAGADIKQMAGKTAMDMYNIDQFSTWDTIKKTKKPLIAAVSGFALGGGCELVMLCDMIVASETARFGQPEIKIGVMPGAGGTQRLTRAVGKALAMEMVLTGRFITAQEAARAGLINRVIPVELFLQEAIRLATEVAALSPLAVKMAKESVLKAFDSSLEEGLHFERKNFYLLFASEDQKEGMQAFVDKRSPVFKGK

SEQ ID NO: 115 - protein, 258 aa, Megasphaera elsdenii
  MISC_FEATURE (1)..(258): DSM 20460
MYTLGIDVGSSSSKAVILEDGKKIVAHAVVEIGTGSTGPERVLDEVFKDTNLKIEDMANIIATGYGRFNVDCAKGEVSEITCHAKGALFECPGTTTILDIGGQDVKSIKLNGQGLVMQFAMNDKCAAGTGRFLDVMSKVLEIPMSEMGDWYFKSKHPAAVSSTCTVFAESEVISLLSKNVPKEDIVAGVHQSIAAKACALVRRVGVGEDLTMTGGGSRDPGVVDAVSKELGIPVRVALHPQAVGALGAALIAYDKIKK

SEQ ID NO: 116 - protein, 428 aa, Megasphaera elsdenii
  MISC_FEATURE (1)..(428): DSM 20460
MSEEKTVDIESMSSKEALGYFLPKVDEDARKAKKEGRLVCWSASVAPPEFCTAMDIAIVYPETHAAGIGARHGAPAMLEVAENKGYNQDICSYCRVNMGYMELLKQQALTGETPEVLKNSPASPIPLPDVVLTCNNICNTLLKWYENLAKELNVPLINIDVPFNHEFPVTKHAKQYIVGEFKHAIKQLEDLCGRPFDYDKFFEVQKQTQRSIAAWNKIATYFQYKPSPLNGFDLFNYMGLAVAARSLNYSEITFNKFLKELDEKVANKKWAFGENEKSRVTWEGIAVWIALGHTFKELKGQGALMTGSAYPGMWDVSYEPGDLESMAEAYSRTYINCCLEQRGAVLEKVVRDGKCDGLIMHQNRSCKNMSLLNNEGGQRIQKNLGVPYVIFDGDQTDARNFSEAQFDTRVEALAEMMADKKANEGGNH

SEQ ID NO: 117 - protein, 372 aa, Megasphaera elsdenii
  MISC_FEATURE (1)..(372): DSM 20460
MSQIDELISKLQEVSNHPQKTVLNYKKQGKGLVGMMPYYAPEEIVYAAGYLPVGMFGSQNPQISAARTYLPPFACSLMQADMELQLNGTYDCLDAVIFSVPCDTLRCMSQKWHGKAPVIVFTQPQNRKIRPAVDFLKAEYEHVRTELERILNVKISDLAIQEAIKVYNENRQVMREFCDVAAQYPQIFTPVKRHDVIKARWFMDKAEHTALVRELIDAVKKEPVQPWNGKKVILSGIMAEPDEFLDIFSEFNIAVVADDLAQESRQFRTDVPSGIDPLEQLAQQWQDFDGCPLALNEDKPRGQMLIDMTKKYNADAVVICMMRFCDPEEFDYPIYKPEFEAAGVRYTVLDLDIESPSLEQLRTRIQAFSEIL

SEQ ID NO: 118 - protein, 258 aa, Chloroflexus aurantiacus
  MISC_FEATURE (1)..(258): strain ATCC 29364 / DSM 637 / Y-400-fl
MSEESLVLSTIEGPIAILTLNRPQALNALSPALIDDLIRHLEACDADDTIRVIIITGAGRAFAAGADIKAMANATPIDMLTSGMIARWARIAAVRKPVIAAVNGYALGGGCELAMMCDIIIASENAQFGQPEINLGIIPGAGGTQRLTRALGPYRAMELILTGATISAQEALAHGLVCRVCPPESLLDEARRIAQTIATKSPLAVQLAKEAVRMAAETTVREGLAIELRNFYLLFASADQKEGMQAFIEKRAPNFSGR

SEQ ID NO: 119 - protein, 258 aa, Ruegeria pomeroyi
  MISC_FEATURE (1)..(258): DSS-3
MAFETIIVEVEDHVALIRLNRPDALNALNTQLLGELCTALEEADGNDKVRCIVITGSDKAFAAGADIREMSQKTYVEVYSENLFAAANDRVSAIRKPIIAAVAGYALGGGCELAMLCDFIIAADTAKFGQPEINLGVIAGIGGTQRLTRLVGKSKSMDLNLTGRFMDAEEAERAGLVSRVVPAKKLVEEALSAAQKIAEKSMISAYAVKEAVNRSYETTLSEGLLFERRVFHSMFATEDQKEGMAAFLEKRAAQFRDK

SEQ ID NO: 120 - DNA, 900 bp, Dictyostelium discoideum
  MISC_FEATURE (1)..(900): Slime mold
atgattaatagattattttcaattaataatattaaaaatggatcaaaattttttagttcatcaacaacagttgaaactaaacaaccattagttttattagaaaaacatttagtaaatggaaaatatacaggtattcaaattgttaaattaaataaaccaaaacaattgaatgcattaacatttgaaatgggagttgattataagaaggtggtggatacattagcagaagataaagatttgaaatgtgttgtattgacaggtgaaggtaaggcattttcggcaggtggtgatttagatttcttaattgaaagaactaaagacacaccagaaaacaatcaaagaattatggaaagattctatagaacatttttatatattcgttcattaccagtaccaatcatttctgcaatcaatggtgcagcaattggtgcaggtttctgtttagctttagcaactgatattcgtgtcgttagtaataaagcaccagtgggtttaacattcaccaaattaggtattcatccaggtatgggtgtaactcattcaattacaaatatagttggtcaagatgttgcatcctatatgttattatcaagtgatattatcaaaggtgatgaagctcaaagattaggtttagttttaaaatcggttgaatctgatcaagttttaccaactgctttaaatctcgctgaaacaatctcaaaaaattcaactatcgctgtaaactctacaacaaaaactttacgtaataaatataattcagatttagataaaagtttaactcgtgaagctgatgctcaaagtcaatgttgggcttcaaaagatatagttgaaggtattttagcaattagagaaagtagagatccaaaacataattatttattatttgatgatcaaaaataa

SEQ ID NO: 121 - DNA, 786 bp, Clostridium acetobutylicum
atggaactaaacaatgtcatccttgaaaaggaaggtaaagttgctgtagttaccattaacagacctaaagcattaaatgcgttaaatagtgatacactaaaagaaatggattatgttataggtgaaattgaaaatgatagcgaagtacttgcagtaattttaactggagcaggagaaaaatcatttgtagcaggagcagatatttctgagatgaaggaaatgaataccattgaaggtagaaaattcgggatacttggaaataaagtgtttagaagattagaacttcttgaaaagcctgtaatagcagctgttaatggttttgctttaggaggcggatgcgaaatagctatgtcttgtgatataagaatagcttcaagcaacgcaagatttggtcaaccagaagtaggtctcggaataacacctggttttggtggtacacaaagactttcaagattagttggaatgggcatggcaaagcagcttatatttactgcacaaaatataaaggcagatgaagcattaagaatcggacttgtaaataaggtagtagaacctagtgaattaatgaatacagcaaaagaaattgcaaacaaaattgtgagcaatgctccagtagctgttaagttaagcaaacaggctattaatagaggaatgcagtgtgatattgatactgctttagcatttgaatcagaagcatttggagaatgcttttcaacagaggatcaaaaggatgcaatgacagctttcatagagaaaagaaaaattgaaggcttcaaaaatagatag

SEQ ID NO: 122 - DNA, 468 bp, Clostridium difficile
aatagtaaaaaagtagtgatagctgctgtaaacggatttgctttaggtggatgtgaacttgcaatggcatgtgatataagaattgcatctgctaaagctaaatttggtcagccagaagtaactcttggaataactccaggatatggaggaactcaaaggcttacaagattggttggaatggcaaaagcaaaagaattaatctttacaggtcaagttataaaagctgatgaagctgaaaaaatagggctagtaaatagagtcgttgagccagacattttaatagaagaagttgagaaattagctaagataatagctaaaaatgctcagcttgcagttagatactctaaagaagcaatacaacttggtgctcaaactgatataaatactggaatagatatagaatctaatttatttggtctttgtttttcaactaaagaccaaaaagaaggaattgtcagctttcgttga

SEQ ID NO: 123 - DNA, 777 bp, Clostridium pasteurianum
atgggaaatattatctttgaagaagaagatggaatagaaaaagttacaattaacagacctaaagctcttaatgcattaaatagtgaaacattaaaagaacttggtacagtaataaatgacatatctgtaaacgatggaataaaagctgtaataataacaggttcgggatcaaaagcttttgtagctggtgcagatatagctgaaatgagtactctaaattcaatagaggcaacaaatttttcaagacttgcccaaaatgtattttcacaaatagaaaatctacctaaattagtagtagcagcagttaacggttttgctcttggaggaggatgtgagcttgcaatggcttgtgatgtaaggtttgcttcaaaaaaagctaaatttggtcaaccagaagttaatttaggaatattgccaagtttcggaggaactcaacggcttccaaaattggttggaaagggaatagcaaaagaattgatattttctacagatatgattactgccgatgaagcttatcgtataggacttgctaataaagtctatgaacctgaggaattattagtaaaatcacaggagtttgctgaaaaggtaatgactaaatctccatggggtgttaaattagcaaaagcatgtataaataatggattagatgtagatttggaagcaggacttaaatatgaagcaaattcatttggtctgtgtttttcaacggaagatcaaaaggaaggtatgaaagcatttttagaaaaaagaaaagcagacttcaaaggactttaa

SEQ ID NO: 124 - DNA, 789 bp, Clostridium pasteurianum
atggattttaataatattatccttgaaaaagaggaaaaaattgccgtagttacaattaatagacctaaagctcttaatgctttgaacagtgaaacgttaactgagcttgattctgtaattgatgaaattgacaaagataatgaaattttagcagtggtattaacgggagcgggaaaatccttcgtagctggagccgatatatcagaaatgaaagacatgaatgtagtagaaggaagaaaatttggaatactaggtaataaggtgttcagaaaacttgaaaatttagaaaagccagtaatagcagcccttaatggatttacattgggtggtggttgtgaaattgctatgtcttgcgatataagaatagcttctactaaggcaaaatttggacagccagaggtacagcttggaataactccaggttttggcggtactcaaagattagctagattaataggcccaggagctgcaaaggaacttatatatactggaaaaattataaatgctgaagaggcctatagattaggacttgttaatagagttatagaaccagaaactttattagatgaagcaaaacaattggcaaatactatagcagccaatgcacctatagctgttaagttggctaaatcagcaataaatagaggaattcaaactgatattgatacaggtgtgtcaattgaatcagaagtatttggagcttgtttctctacagaagatcaaaaagaaggtatgaatacattcttgaatgataaaaaatatttaactggtaattttaagaataaataa

SEQ ID NO: 125 - DNA, 783 bp, Megasphaera elsdenii
atggattaccagaacattatttttgctgtagaagacggtattgcaacgattacgatcaatcgcccgaaggctctgaacgctttgaaccaggctacggtcagcgaattgaaagacgtcgttgaaaagattgcagctgataaagctatcaaagtcgtcatcatcaccggtgcaggcgctaaatccttcgtcgctggcgctgacatcaaagaaatggcttccaagaacgctgctgaaggccgcgaatggggccagttcggtcagaacgtcttcacggaaatcgaaaacctgccgcagcctgtcatcgcagctatcaacggcttcgctctcggcggcggctgcgaactctcctgcgcttgcgatatccgctatgcagctgaaaacgctaaattcggccagccggaagtcggcttgggcatcactccgggctttggcggcacgcagcgcctgacccgtgtcgtaggccgcggccacgcgaaagaactcatctacacgggcggcatgatcgacgctgaaaaagcaaaagctatcggcttggtcaatgaagtcttcccgcaggaagaactgatgccggctgctgttaaattggctaagaagatcgctaagaacgctcctattgcagtacagctctccaaagctgccatcaaccgcggcatcaactgcgacgtcgtaaccggtatcgcttatgaagctgaagtcttcggcctctgcttctccacggctgaccagaaggaaggcatggctgctttctgcgaaaaacgcaaagcaacgtttgaaggtaaataa

SEQ ID NO: 126 - DNA, 780 bp, Metallosphaera sedula
atggaatttgaaacaatagaaactaaaaaagaaggaaacttgttctggattacgttaaatagacccgataaactaaacgcactaaacgctaaattacttgaggagttagatagggcagtctctcaggcagagtctgacccagagattagggttatcatcattacagggaaaggaaaggccttctgcgcaggggctgacataacccagtttaaccagttaaccccagcagaagcctggaaattctctaagaaaggaagagagatcatggacaagatagaggcactgagcaaacccaccattgccatgatcaatggatatgcccttgggggtggactagagctagccttagcctgtgatataaggatcgcagcggaggaggcccaactaggccttccagagataaacctagggatatatccggggtatggggggactcagaggttaaccagagttataggaaagggaagagccctggagatgatgatgacgggcgatcgtattcctggtaaggatgctgagaaatatggtctcgtgaatagggttgtccccctagctaacttggagcaagagacaaggaagctggcagaaaagatagccaagaagtctcctatctctctcgccttaatcaaggaagttgtaaacaggggactagactctcccctactgtcaggtctagcgttggaaagcgtaggatggggagtcgtgttttctacggaggacaagaaggagggggtaagtgccttcctggagaagagagagcctacgtttaagggaaaatag

SEQ ID NO: 127 - DNA, 779 bp, Clostridicum kluyvery
atggaatttaaaaatatcattcttgaaaaggatggaaatgtggcttcaataacgttgaatagacctaaggcattaaatgcattaaatgcagcaactttaaaagagatagatgccgcaataaacgacattgctgaagatgataacgtatatgctgtgataattactgggtcaggtaaagcttttgtagcaggagcagatatagctgagatgaaagatcttactgcagttgagggaagaaagttttcagttcttggcaataaaatatttagaaaattagaaaatttagaaaaaccagttatagcagctataaatggatttgcactgggtggtggctgtgaattgtcattgtcttgcgatataagaatagcttcatcaaaggctaagtttggtcaaccagaggttggtcttggaattactccagggtttggaggtactcaaagacttgcaagagcaataggcgttggtatggctaaggaacttatatataccggaaaagtaattaatgctgaagaggcattaagaataggtttggtaaataaagtagttgagccagataaattattggaagaagctaaagctttagtagatgctattattgttaatgcacctatagctgttagaatgtgtaaggctgctataaatcaaggacttcagtgtgatatagatacaggtgtagcttatgaagcagaagtatttggggaatgttttgctacagaagatagagtagaaggaatgacagcatttgtagaaaaaagagacaaggcttttaaaaataagta

SEQ ID NO: 128 - DNA, 1509 bp, Sulfolobus tokodaii
atggcaattagaactggagagcaatatttagattctataaaaattagaaataaggctgaaatttacgtaatgggaaaagaagtaaaggatgtaaccactcatcccttcttgaaaccttctgtaatggcatttaaggcaacatttgatgctgcttgggaagaggacacaaaagaattagccagagcatggagtcctttcataaatgaagaagtgaatagatttaatcacatacacaggtcaccagaagacttagctgctaaagtgaaattactgagaaaattaagccataagaccggtgcatgtttccaaagatgtgtaggatgggacgctctgaacactttgtggattatgacgaatataatggctcaaaaaggtaaaaaagaatataaggatagatttgtcgaatacttaagttacgtccaaaagaaggatttagcattagctggtgctatgacagatgcaaaaggtgtaagaacattaaaaccgcatcaacaaccaaataagaacgcttatgttagaattgaggaagttaccaaagacggtatttatgtttctggtgcaaaggcaaatattactggtgtagctgcaacagaagaaattgtggttttacctactagggctatggggccagaagataaagattatgctgttgcattttcaataccgacagatactgagggtataaaaattatagttggtagacaattaaatgatgctagaagattagaaggtggtgacatagatgctttaccgtacttctataaccacgagggtttagtaatctttgaccatgtttttgtaccaatggatagagtattcttaatgggagaatacgagtttacttcacaattagttgaagtattctcagcatatcatagacaaggatatggtggttgcaaggctggtttaggagatgtaattattggtgcatcaatgaatttagcaaaacaattaggagtagaaaaagcttcacatgtacaagaaaaactaacggaaatgatattcttaactgagaccatgtattctgcaggaattgcagctagtttaaatgcagttaaggtctgcgataattgttggtgggttaatcctatgcacgctaatgttacaaaacatttagtagctagatttccagcccagatttctcagttatctatcgatattgcaggtggaataataggtactgcaccaagtgagtgggatctcaagaatcctaaattaagagaatatattgccaaatacttacaaggtgttgagggttatacagctgaagatagattaagaatggttagattactggaaaacgttagtctgggtgttgcattccaaattgaatctgtacacggtgcaggaagtccagcagcacaaagaataatgtttagtagactttatgacttaaactatgctgaggaagtcgcaaagaggttagctgggaagaagactgatttacagtggaaacctaaagcagagccttggagagaaagtgagacagaaaaattagtaaaaagttaa

SEQ ID NO: 129 - DNA, 1452 bp, Geobacter metallireducens
atggcactaagagatgggaattcctaccgggaaagccttcgggcgctcaatatcaaagtctatgcctttggagagaagattgacagcatagtagatcacccattgttccagccccatatcaatgcggctgcattgacgttcgacttggcccatgatccgaccacggaagcgctcgtcacagccacctcacacctgacggggagtaaaatcagccgcttcacccatatccaccagagcaccgacgatctcataaaaaaggtgaagatgttgcggcttattgcagggaagacgggaagttgctaccagcgctgtgtggggtgggatgccctgaacgctaactatacggtaacctatgagatggaccaggagcttggtaccgactatcaccagcgttttaggcgttacctcgaatatatacaggacaatgacctgatggtggcgggagcaatgaccgatcccaagggggacagggggctgcctccggcaaaacagaaagacccggacatgttcgtgcacgtggtggcaaagaatgacaaggggatagtcattcgtggggcaaaggttcaccagaccggaattgtcaattcccatgaaatgctgattatgccaaccatggccatgggggaggaggacggcgactatgcggttgcctgtgctctccccacggattcccccggtgtcatccatatctttggtcgtcaaaccaacgatacacgccgtctggaaaagggagaccttgatcagggtaatgctgagtatggaactgtcggaggcgaggctttgaccatacttgaagatgtcttcgtcccgtgggaacgcgtcttcatgtgcggagagtacaagtatgcggggctgctggttgagcgtttcgcgagctatcatcgacagaactatggtggatgcaaggcaggcgtgagcgatgtgatcatcggcgcaactaccgctatggcagagtacaacggagcagccaaggcttcccacgtgcgtgacaagatcgtggagatggtccacctcaccgagaccctttattgcggttccatcgcctgctcctgtgagggtgctcccacgccgtcaggggcctatttcgtcaatcccctgctggccaatacggttaagcagaacgtgacccgtttcatctatgagattgcacgcctttcccacgatatttccggtggctgcatggcaaccatgccttcggagaaggatctgcaccacgatgagatcggcaaatatgtagagaagtatttccggggggtggacgaagctcccactgaagagcgcatgcggatggcccggctcgttgaaaatatgacgggcggcacggctttggtggaaagcatgcatggtgccggctctccccaggcgcagagagtcatgatcctccgccaggcaaatctcggccataaggtaaagcttgccaagaaactggccggcataaaggaagaaaaatag

SEQ ID NO: 130 - DNA, 1392 bp, Sulfolobus solfataricus
atgagatcaaaagaagatttcctaaagtccttaaaagatggaagaaatttgtattatagggggaagttagtagaagatataacaacacatcagatcttaaagacagccgcattgcacgcagctaagttatatgaatacgctgatagagtctatgaggataataaaatgggaaaaatgagcaagttctttaaggtaccttggacatctcaagatttgctagatagacataaactaatttacgatttaacgatgtattgtaatggggtatttaacatttcacaagcaataggaagtgatgcgatctttgcccttatgatcacggcaaaacaagttgatagaaaatacggaactgattactcaaaacgtgttgaaaaatattttgagagagttgctaaagaagatttaacgttagccactgcccagactgacgttaagggagatcgaagtaagaggccttctgaacaagttgatccagatatgtatgttagagtagttgatgtgaaaagcgatggaatagttgttagaggagcaaaggctcatacaactcaatctgcggtatctgatgagattattgtcataccaaccagagtaatgagggatagcgataaagattacgcagtagcctttgcggttccagctaatactaaaggtttgaagatgtatattagaccaattgatgaaattgagggcaattcctcctcagtactcagtagaaaagattatgagctagaaacattaaccgtcttcaacgacgttttcgttccttgggatagggtatttttatttaaggaatacgactacgctggaacattggctatgctatttgcaaccttccataggtttactgcattatcgtataggtcagcgaccatgaatctatatttgggagcatctaaagtggcatctcaagtaaatggcattgagaatgaaaagcatgtgagagatgatatagttgatataattctctacaaggaaattatgaggagtagcgcgatagctgcggctgtgtatccagtaaacatggagggtatagctgtgcccaacccgctttttactaatgttggtaaattatactccaatatgcatttccatgatgttgtaagagatttaattgacattgctggggggataatagctactatgccctctcaagaagatttggaaagtgatgaaggaaagaatattgttaaatatttaaggggctcagttgatggagaggaaagagcaaaagtgttaaaactagctaaggaattaggggctagtacgtttactggctatttgctaactggtatgatacatgcggaaggttctatggaagctagcaaaatagagctattcagaagttataattttaaggaggccgagaacttagttaaaagggtattaagctag

SEQ ID NO: 131 - DNA, 1440 bp, Syntrophobacter fumaroxidans
atgggactcaaaacgaaggcggaatatatagaatccttgcgaggcatgaagccgacggtctacatgttcggtgagaagatcgaaagcgttgtggacaatccacgcctgcgagcgggcatcgaggcgacgggggcgacgtacgaactggcagagacggaggagtatcgccctctcattgtgactgaaagtcccctcattcacgaacccgtcaaccggtatacgttgcccccgtcgtccatcgcggacctcgtcgccagggtgaagatcaatcgtctcatgggcactcgtgtcgggacctgctttcaacggtgcacggggctggactgcctgtccgccctttccatcgtgacctacgacatcgacgccaagcattccaccccttacttcaaacggttcatcgagtttctgaagcatgttcagaaaaacgacctgacctgcaacgccggcgtgaccgacgtcaagggcgaccgttccctggccccccacgagcaggaagacaaggacatgtacgtgagggtcgtggaacgcaatgcggacggcatcgtcgtgaggggcgccaaggcgcaccagaccggttccctctcctcgcacgaaatcatcgtcctgccgacgcgtgccctgcgaaagggcgacgaggactacgcgctcgcttttgccatccccaacgacactcccggcctgattcacgtcgtgggccgatcgagcctcgacacccgccagctggacggctgcgacctgggcaaccttcactattccaagtactgcccgaccgtgatcttcaaggacgtgttcgttccctgggagcgggtcttcatgtgcggcgaggtggaattcgccgtggagatggtgaaccgcttttcggcttatcaccgccagagccacggcggctgcaagtcgggcaagatcgactgcatggtcggagcggccctcaccatgatggactacaacgggacggagaaggccgggcatctcaagcagaaggccatcgagatggtccaccgggcggaaaccctctacggctgcagcctggccgcgtcctacgagggcaaaaaagaaccttccggaacctacttcatcgacacggtgctggccaatgcgtccaagatccacgaaggcaaggaaatgagcgaggccggccgcctgctggtggacatcgccggaggcttcgtggccgatctgccttcggatcgcgacctggccattcccgaagtcggggaactgctgaaaaaatacctgaagggggtggcgtcggtgccggtggaagaccgcgtcaaaatgtaccggctgatcgaaaagctcgtcatggaaagcgccgatacgatttcggacatccatggaggcggttctcccgaggcccacaggatcacgatcctgcgggaaagcaacctcaaggccaagaaggacgcggccaagcggttggcgggaatcgaatcgaagtag

SEQ ID NO: 132 - DNA, 1461 bp, Porphyromonas gingivalis
atgatgactagcgaacagtacgtagaaagtcttcggaaacttaatctgaaggtttacttcatgggtgaaaggatcgaaaaccctgtagatcatcccatgattcgtccctcaatgaattcagtagctatgacttataagcttgctgagatggacgaatacaagcatttaatgacagcaacttcaaacttgactggtaagcaagtgaatcgtttctgccatctacatcagagcacagaggatctgaaagacaaagtgaagatgcagcgtctcatgggacaaaaaacagcttcatgcttccagcgttgtgtgggaatggatgcattcaatgccatctattctactacttacgaaatggatcaagctctgggtaccacttatcacaagcgtttcatcgagtacatgaaatatgtacaagacaacgacttggtcgtagatggagccatgacagaccccaaaggggatcgcggtttatctccctcagaacaagccgatccggatctttatctgcacattgttgaagttcgtgaagatgggatcgtcgtttccggtgcaaaggcacaccaaaccggagcagtcaattcgcacgagcatctgatcatgcctacgatcgctatgcgcgaagctgatgctgactatgccgtttcttttgccgttcccagtgatgcagagggcgttattatgatctatggccgccagtcatgcgacactcgcaaaatggaagaaggggcagacattgacctcggcaactctgaattcggcggacatgaagctcttgttgtattcgaccgcgtattcgtgcccaatgaccgcgtgttcatgtgcaaagaataccagtttgcaggtatgatggtagaacgtttcgccggataccaccgtcagtcttatggaggatgtaaagtaggtgttggtgatgtacttatcggtgcagctgctctcgcagcagactacaatggagttcctaaggcatctcacattaaggataaactcattgagatgatccacctgaatgaaaccctttatgcttgcggtattgcatgctcttcagagggaactcagatgaaagccggcaactatatgatcgatttgctgttagctaatgtttgtaagcaaaatatcacccgccttccttatgaaatagctcgcttggcagaagatattgcaggaggtttgatggtaaccatgccttctcaacaagacttccgccatccggaaataggcccgatcgtaaagaaatatcttgcaggggcaacaggcaaatcgacagaaaaccgtatgcgtgttctgcgtttgatagagaatatcacgctgggaacagctgccgtcggttatcgaaccgagtctatgcacggagccggatctcctcaagctcagagaatcatgatcgctcgtcagggagatcttgagggcaagaaaaagcttgcacgggcgattgctcatatcgacgaatcactcgataagtaa

SEQ ID NO: 133 - DNA, 1590 bp, Polynucleobacter necessarius
  MISC_FEATURE (1)..(1590): subsp. Asymbioticus
atgagtcaaagcacctcccagttcatgaatagcaaagactatcaagagtcattgcgctcactaaagccaactgtctatgtcgatggtcgattgatcgaatccgtcgccgatgagccttctcttcgccctggagtccaagccttaggagtgacttatgacatggtccatgacccagcgctagcaccgctcatgttggctgactcgaatggcactcctgtaccaagaatgctgcacattaatcagtcttctggagatctcttaaataaattagaagcggtacgtgtactctgccaagaaactggatgtgcccaacgctatttagcccatgatgcgttaaatgcgattgcacaagtttctgcgcgcattgatgatgccaaaggaagtaatgagcatagtgctaaattttctgagtatctatcgcatgtacaaacgaaggacttggcattaggcattgccatgacagatgcaaaaggagatcgctcccgcagacctcatgagcaagaaaatccagatacttacgtacatatcgtttctcaagatgctaaaggggtcgtgatctcgggtacaaaagcgattgtgactggcgccccttacatgcatgaattcttagtcatgccaggtcgcaatatgactaaagaggatgcagcctttgcgatttgctgtgctgtccctgtggatgccaaaggtattacgattgtggcacgcccagcgggacgcccaggcgacaaggtcgagcatggtaaaccgatattttctagtaaatatggtcaatcgactggggtagtgatattcgataaagtattcgttccctgggatcgtgttttttatgctggcgaatgggaacactctagcgtgctgacttataactacgccacccatcatcgtcatagctgcatcgcggcgcgagcaggctttggagatctgttaattggtgctggcgctttaatgtgcgaagcgaacggattggatccagcaaccaaatctaatttacgtgatccgatggttgaactcattaagatcactgaaggattttatgcttgcggtgtggctgctagcgtctatggaacgcaagatccgtacagtaaatcatttatgcctgagccggtattttctaatatcggaaaactcttattagcaacgcagatttatgacatgcatcgcttggcacatgaagtatcgggaggattaatcgtagcgttgccaggaccagacgaagatcacaacccagcaactgcagccactttggcagaggtgttacgagccaatccagccgtcccttatgacaagcgaattgaagttgcacggtttattgaagatctcacagcgtcttatcaaggcggttggtattccgtcattagcctacatggtggcggctctccagcagcaatgaagcaagaaatctatcgtcagtaccctattggcaataaagtagagctagtggaacgtttattagatcgcggagtgctgactagtagcgaagagcgggcgattacgaaaaataaacaacctgggcgctgctgcgatcaaggctgtagcgcgccaggacaagcagtgatggtacctttgccagagcctggcagaagaacttaa

SEQ ID NO: 134 - DNA, 777 bp, Gordonia terrae
  MISC_FEATURE (1)..(777): C-6
gtgaccgaacaccagaccatcgtcgtcgagaccagcggccgggtgggcatcatcaccctcaaccgcccgaaagcgctgaacgcgctcaacaccgagttgatgaacgaagtggtcggcgccgtcaaggagttcgacgtcgaccaggggatcggcgccatcgtgatcaccggttcggagaaggcgttcgccgcgggcgccgacatcaaggagatgtcatcgaagtcctacgcggatgtggtgaacgagcagttcttcggcgcctgggatgagctgtcgcgggcgcgtacgccgatcatcgccgcagtgaccggctacgccctcggcggcggctgcgaactcgcgatgctgtgcgacaccatcatcgccggcgacaacgccgtcttcggtcagcccgagatcaacctcggcgtcatccccggcatcggtggttcgcagcgcctcacccgcgccgtcggcaaggccaaggcgatggacatggtgctcaccggccggcagatgaaggtcgacgaggccgagcgtctgggcctggtctcgcgggtggtgcccaaggaggactgccgcgccgccgcgatcgaagtcgccgagataatcgcctcgaagtcgctgatcgccgccgcggccgccaaggacgcggtcaaccgtgccttcgagtcgagcctggtggagggtgtccgcgccgagcgcgcgctgttctactcgacgttcgcgaccgacgaccagaccgagggcatggccgccttcgtcgagaagcgggacccgaacttcacccaccgctga

SEQ ID NO: 135 - DNA, 777 bp, Halalkalicoccus jeotgali
atggcagacagagtactcatcgaacgagagaatgacatagcgacgatcatcgttaatcggcctgagaagcgtaatgcgatggatatcccgacgcgaaaagccctctatgccgccttcgaagaggttagcgaggatgacgatgtgcgggcaatcgtgctccgcggagcaggagatgggtcgtttatcgccggtggcgatattgattctttcgccgacttcgaccacatggacggcatggagtacagcgagaagtacgcccaagggctgtacaactatgttgcggaccgccacaaaccaaccatcgccgcggttgacggctacgctctcggtggaggcaccgaaatcgccctcgcttgcgacattcgcctcgccacggacgacgcgaagttcggcctgcccgaagtcggcatcggcgtcatcccagccggtggtggaacacagcgactcgttcaagtcgtcggagccgggcttgcaagcgaacttatcctcactggccgcattatcagcgccgacgaggcaaagagaattggtcttgcaaaccatgtctacgccgccgaggaattcgataatgaagtccgagccatggccgaagatcttgcctcgaaggcgcctgtcgcccagcgacttgcaaaagaatccatccgacgtagccttgatatcgacgccggccttgaatacgagcgactggccggagcgtttctgttcggcaccgacgaccagaaagagggtgcaaacgccttccttgaggaccgagagccgaagtaccgaaaccggtaa

SEQ ID NO: 136 - DNA, 774 bp, Carboxydothermus hydrogenoformans
gtggaatttgaaaaaattaaatttgaggttacggacggttatgccgttatttacctaaacaacccgccggtaaatgctcttggccagaaagttttaaaagatttacaaaaagctttgcaggaaattgagaaaaatcccgagattcgggcggtaataattagcggggaaggtagcaaggttttctgtgccggggcagatatcacggaatttgctgaccgggctaaagggattttaccggaagtggaaggaagtgttcttttccggcaaattgagcttttccccaagccggtgattgctgcgctgaacggtagctcctacggcggaggaaccgaattagcgataagctgtcacctgcgcattttagcagatgatgcttccatggctttgcccgaagtaaaactgggcattatccctggctggggaggtacccagaggttaccccggttaattggtaaaaccagagccctggaagcaatgcttaccggagagccaataacggcagaagaagccttaagctacggtctggtaaacaaagtcgtacccaaagaccaggtactaacagaagcccgggcgctggcagctaagcttgccaaaggggcgcccatcgctatgcgggaaattttaaaggcggtaactttagggctggatacttcaatagaagaaggtttaaaaattgagaaagaaggttccaaagtggcgtttagcagtgaagatgcggtggagggaagaactgctttctttgaaaaacggccgccgaattttaaaggccggtaa

SEQ ID NO: 137 - DNA, 774 bp, Thermomicrobium roseum
atgagcgtgcgtgtcgagcgggagggggcgatcaccctcgtcacggtcgagcgcccggaacgactgaacgcgctcgataccgcgacgttgcgtgccttactcgcggcagtgcaggaactggcaacggaggaggcgatcgctgtcgtcgtcctcaccggggcaggcgatcgcgcgttcatcgccggagccgatatcagcgagatggtagagaagtcgccagccgaggcgctcgccttcgccgagttgggacacgccgtttgccgggcgatcgaggaagcgccgcaaccgtacatcgcagcggtcaatggctacgcgctaggaggcggctgcgagatcgcgctggcgtgcgatatccgcctcgccagcgagcgcgccgtcttcgcccagccggaagtaacgctgggtattccaccaggctggggcggatcgcaacggctgccgcgcgtcgttcctcctggtatcgcgcgcgagttgctctatacggggcgccgcgtcgatgcgcaggaagcactgcggatcgggctcgtcaatgccgtctatccggctgaccaactcctcgagcgagctcgggaactggcgaaccggatcgcggccaacgggccactcgcggtccgcttgaccaaggcggcggttcgcttcggtctcgagcaggggctggaagctggactgacctacgagcggcaggtgttcgcgtacgcgttcaccaccgaggatcagcgggaggggatgcgggcatttctggaaaagcgtcgtccggcttttcgcgggcgctga

SEQ ID NO: 138 - DNA, 825 bp, Methylobacterium extorquens
atgaacgctgacgccgagaccgcctcgaccgacgaactgctcttcgcggtggatgcggcgggcatcgcccgcatcaccctcaaccggccgaaggcgcgcaacgcgctgaccttcgcgatgtatcgcgggctggtggagttgtgcgagcggatcgaggcggaccacgcgatcaaggcggtgatcatcaccggcgccggggacaaggcgttcgcggcgggtaccgacatcgcccagttccgtagcttcagcaaaccggaagacgcgatcggctacgagcgcttcatggaccgggtgctcggcggcctggagcgcctgcgggtgccgaccatcgcggcggtcgccggagcctgcaccgggggcggtgcagcgatcgctgcggcctgcgacatgcgcatcgccagccgcgacgcccgcttcggcatccccatcgcccgcacgctcggcaattgcctctcgcagaacaccctgaggcggctggcgaacctcattggggcgccccgcgtgaaggacattctgttcaccgctcggctcgtcgaggcgcaggaggctctggcgatcggcctcgtcaacgaggtggtcgaggatgccgcggccgtcgcggcccgagcggatgcgctggccaccctgctcgcgagccacgcgcccctcaccctccaggccaccaaggaaggcctgcgccgcatcggcgaggagggcgcggcggaggccgccgagggcgagcggcccggcgacgacctgatcgtgatgacctatatgagcgcggatttccgggagggcatggaagccttcctgggcaagcgcccgccgaacttcaaagggcgctga

SEQ ID NO: 139 - DNA, 1224 bp, Clostridium sporogenes
atgagtgatagaaataaggaagtaaaagaaaaaaaggcaaagcattatcttagagagattactgcaaagcattacaaagaagctctcgaagcaaaagaaaggggagaaaaggttggttggtgtgcatctaacttcccacaagaaatagctacaacattgggggtaaaagttgtttatccagaaaatcatgcagcagctgtagcagctagagggaatggacaaaatatgtgtgaacatgctgaggctatgggtttttctaatgatgtatgtggttatgcaagagtaaatttagctgttatggacataggtcatagtgaagatcaaccaatacctatgccagactttgtactttgctgtaataacatttgtaatcaaatgattaaatggtatgagcatatagcaaaaactttagatataccaatgattcttatagatataccatacaatacagaaaatactgtttcacaagatagaattaaatatattagagcacaatttgatgatgcaataaaacaattggaagaaataacaggcaaaaaatgggatgaaaataaatttgaagaagttatgaaaatatcccaagaaagtgcaaaacaatggttaagagcagcatcctatgcaaagtataaaccttcaccatttagcggatttgatttatttaatcatatggctgtagcagtttgtgcaagaggtacacaagaagctgcagatgcatttaagatgttagcagatgaatatgaggagaatgtaaaaactggaaaatccacttataggggagaagaaaaacaacgtatattatttgaagggattgcctgttggccatatttgagacataaattaactaagcttagtgaatatggtatgaacgtaactgcaactgtatacgcagaagcctttggtgttatatatgagaatatggatgaattaatggctgcttataataaagttcctaattcaattagttttgaaaacgcattaaaaatgagattaaatgctgttacaagcactaatacagaaggtgctgttattcatataaatagaagctgtaaattatggagtggatttttatatgagctagcaagaagattagaaaaggaaacaggaattcctgtagtatcatttgatggggaccaggcagacccaagaaatttctcagaagctcaatatgatactagaattcaaggacttaatgaagtaatggttgctaaaaaggaggctgaataa

SEQ ID NO: 140 - DNA, 1125 bp, Clostridium sporogenes
atgtcaaattcagataaattttttaatgactttaaggatattgtagaaaatcctaaaaaatatataatgaagcatatggaacaaactggacaaaaggctataggatgtatgccattatatactcctgaggaacttgtattagctgctggaatgtttccagtaggggtatggggaagcaatacagaactttcaaaagctaaaacatatttcccagcatttatttgttcaatattacaaacaacattggaaaatgcattaaatggagaatatgatatgttatctggtatgatgattacaaattattgtgattcattaaaatgcatgggacaaaattttaaactaaccgttgaaaatattgagtttatcccagtaacagttccacaaaatagaaaaatggaagctggaaaagagtttttaaaaagtcaatataaaatgaatattgagcaattagaaaagatttctggtaataaaataacagatgaatctttagaaaaagctatagaaatatatgatgaacacagaaaagtaatgaatgacttttcaatgttagcatcaaaatatccaggtataataacaccaactaaacgtaattatgttatgaaatctgcttattatatggataaaaaagaacatactgaaaaagttagacaattaatggatgaaattaaagctatagaaccaaaaccatttgaaggaaagagagttataactacaggtataattgcagattcagaagatttacttaaaatattagaagaaaataatatagctatagttggtgatgatatagcacatgaatctagacaatatagaacattgactccagaagcgaacacaccaatggataggttagctgagcaatttgctaatagagaatgtagtactttatatgatcctgaaaagaaaaggggtcaatatatagtagaaatggctaaagagagaaaagcagatggaattatatttttcatgacaaaattctgtgacccagaggaatatgattatccacaaatgaaaaaggattttgaagaagcaggcattccacatgtactaatagaaactgatatgcaaatgaaaaattatgaacaagctagaactgcaattcaggctttttcagaaacactttaa

SEQ ID NO: 141 - DNA, 795 bp, Clostridium sporogenes
atggcagacatttatactatgggtgtagacataggttcaactgcatcaaaaacagtagtattaaaaaatggtaaagaaattgtaagtcaagcagtaataagtgtaggggccggaacaagtggccccaagagagctatagattctgtattaaaagatgctaaattatccattgaagatttagactatattgtatccactggatatggaagaaatagtttcgattttgctaacaaacaaatttctgaattaagttgtcatgcaaaaggggtctatttcgataacaataaagctagaacagttattgatataggcggacaagatattaaagtattaaaattagcggatagtggaagacttttaaactttataatgaatgataaatgtgctgcaggaacgggacgatttttagatgtaatgtctagagtaatagaagttccagttgatgagttaggaaaaaaagcattagaaagcaaaaatccttgtactattagttctacctgtacagtatttgcagagtcagaagtaatttctcaacttgcaagaggagttaaaactgaagatttgatagcaggaatttgtaaatctgtagcatcaagagtggctagccttgcaaagagaagtggtatagaagaattagtagttatgagtggaggagtagctaaaaatataggtgtagtaaaggcaatggaagcagaattgggaagagacatatatatatctaaaaattctcaattaaatggagcattgggagcaagtctatacgcttatgaaagttttcaaaaagaaaggagctaa

SEQ ID NO: 142 - DNA, 1239 bp, Clostridium sporogenes
atggaaaacaatacaaatatgtttagtggagtaaaggttattgaattagcaaattttatagctgctccagcagcaggtagattttttgctgatggtggtgcagaggtaataaaaattgaatcacctgctggagatcctttaagatatactgctccttcagaaggaagaccattaagccaagaagaaaatactacttatgatttggaaaatgcaaataaaaaagcaatagtattaaatcttaaaagcgaaaaaggtaaaaagatattacatgaaatgttagcagaagcagatatattattaactaattggagaacaaaggctttagttaaacaaggattagactatgaaacactaaaagaaaaatatcctaaattagtttttgcacaaataactggttatggtgaaaaaggaccagataaagatcttccaggctttgattatactgcatttttcgctagaggcggtgtttcaggtactctttatgaaaaaggaactgtgcctccaaatgttgttccaggacttggagaccatcaagctgggatgtttttagcagcgggtatggcaggagctttatataaagcaaaaacaacaggacaaggagataaagtaacagtaagtttaatgcatagtgctatgtatggactaggtattatgatacaagctgctcaatataaagatcatggattagtatatccgataaatcgtaatgaaactccaaatccttttatagtttcatataaatctaaggatgattactttgttcaagtatgtatgccaccatatgatgttttctatgatagatttatgaccgctttaggaagagaagatttagttggagacgaaagatacaataaaatagaaaatttaaaagatggacgtgctaaggaagtatacagtataatcgaacaacaaatggttacaaagacaaaggatgaatgggataacatatttagagatgcagacattccatttgctatcgcacaaacttgggaagatttattagaagatgaacaagcttgggcaaatgattatttgtataagatgaaatatccaacaggaaacgaaagagcattagtaagacttccagtattctttaaagaagcaggattaccagaatataatcaatcaccacaaatagcagaaaatactgtagaagttttaaaagaaatgggatatacagaacaagagattgaggaattagaaaaagataaagatataatggtaaggaaggaaaaataa

SEQ ID NO: 143 - DNA, 1107 bp, Lachnoanaerobaculum saburreum
atgtggcattgtttagaaactttaaaaaagattagtgcgtctccaaaggaacagcttaataaataccttgaagaaggaaaaaaagttattgctgttgcaccggtttatacacctgaggagattatccatgcttttggatttgtacctatgggggtatggggcgcagatattgaaattaatgagtcaaaaaaatattatcctgcatttatttgctcaataatgcagacagtattggagctgggaataaagggaaattataacggagttagtgctatagtggttccttcgctatgtgactcattaaaaactttgggacaaaattggaaatatgcggtaaaggacattccttttataccaatgacctatccacaaaatagaaaatctgattatgctgttgatttcacattggagatgtataagagagtgatcagtgatttggaaaatattaccggagaaaagtttgatgaaggtaaactcaaaaacacttatgaaatttataatgagcataatagggttatgagagaatttacaaaagtttcggaagagtatgaagtttcggcaacagatagaagtgcagtatttaaaagtgcttggtttatgcttaaggaggaacatacagaacttgttagggaattgatcgaacttataaaaaaagagggtaaaatatctaagaagctaagaatttatacaacaggaatattggcggatgcaccggatttactcaatatttttgacagcaataatatgcaaatcgtaggtgatgatattgcttatgaatccagacagtatagaacagatatacccgatggaaatggtttatatgctcttgcaaagaagttttcaaatatggacaactgtactcttttatatgataaggataagagaagggttgactttattattgaagaagcaaagaaaaaaagagctgacggaatagtagttcttatgaccaagttttgcgatcctgaagaatttgactatgtgcctataaagagggcggcaaatgaagcaggtattccacatatcaatatagaagtggatagacaaatgaaaaattatcaacaggcaaatactatgttacaaacatttgcagacatgttggtttag

SEQ ID NO: 144 - DNA, 1230 bp, Lachnoanaerobaculum saburreum
gtggaagaagctaaaaaacaaaagcctacagttgatccaaacagcgcaaaggctagattgggcaggatagcagcaaaagcatatagtgactgtgttgaggctaaaaagcgaggagaattggtaggatggtgtgcaagtaattttccggtggagatacctgagaccttgggattgtacgtatgttaccctgagaatcaggcggcaggtattgctgccagaggcggtggagaacgaatgtgcagtgagagtgaaggtgacggatactctaatgatatatgcgcatatgcaagaatttcgcttgcatatatgaagctgaaggaagctcctgaacaggatatgccacagcctgactttgttctatgttgtaataatatatgcaactgcatgattaagtggtatgaaaatatagcaaaagaacttaatattcctatgattatgattgatataccttttaatcctgattatgaagtttcagatgctatgacagcatatatcagaaatcagttttgggatgcaatacatcaattggaggaaattacaggcaaaaaatggagtaatgaaagatatgaagaggtaaggaaaatatcaggaagaagctccagagcatggcttgaggctacagcgactgccaaatattcaccatctccgtttaacggatttgatttattaaatcatatggcggttatggttactgccagaggaaaacttgaagctgcagaagcaatggaaacacttttgcaggagtacaaggataatcatgagaagggagagtctacgttcaagggagaagaaaaatatagaataatgtttgagggtatagcatgctggccatggcttcgtgctactgctacaggacttaagagtcgtggaatcaatatggttacaactatatatgcggatgctttcggatttatctatgatgactttgacggaatgtgcagagcatatgccaatgttcctaattgtatgaatatagagcatgcaagagataagagaataaaactttgtaaggacaatagtgttgaagggcttctcgttcacacaaacaggtcttgtaaactttggtcaggatttatgtctgaaatgagcaggcaaataggtgaagaatgtggtattccggttgtaagctttgatggagaccaagcagatccaagaaatttctcagaggctcaatatgatacgagagttcagggattgacagagataatggaagcaaataaggaaatttaa

SEQ ID NO: 145 - DNA, 771 bp, Lachnoanaerobaculum saburreum
atgtacacattgggtgttgatataggctcaactacatccaaagcggtaatattggaggatggagaaaatatagttgcatcttcaattgttatagcaactgtaggaacggcaggagtagaagaggctgtaaaaaatgtactaaacttttcaaaactcgaactaaatgacattaaagcagtggttgctacaggatatggaagaatgaattatgatgtagcagattacaaggttagtgaattgacatgtcatgcattaggtgtacataaggagttcccgaatgtcagaactgtaattgatatcggaggtcaggatgccaaggtaatatctcttgcggcaaacggtaagatgacaaattttgttatgaatgataaatgtgcggcagggacaggtagatttcttgatgtaatggctaatatattaaatcttgatatacaggatttggaggtggaagccttaaaatcagataatccggcaaatatatcaagtacttgtacagtttttgcggaatcggaagtcatatcacagcttgctacaggaagaaatattcctgatttggttgcagggatatgcaaatctgttgcagtaagggttgccgccctggctaaacgagtaggtatagttgaagaagtgtgtatgagcggcggagtggcaaaaaactcgggtgtgaggaatgctatgagtaaagagcttggtgtagatatagtgtttagtaaggatgctcaacttatgggagcacttggagccgcaatatacggttttaaaaagttataa

SEQ ID NO: 146 - DNA, 795 bp, Peptostreptococcus stomatis
atgagcagtgtatacacaatgggtattgacattggatcaacatcatcaaagtgtgtgataatgaaggatggtaaggaaattgtaagtgaaggtgtagttagcttgggtgctggaactaagggttctgacctagttattgaggaagtgcttggtaaggcaggaatgactttcgatgaaatagacctaatcgtatcgactggatatggtagaaatagctatgaaagagctgccaagactgttagtgagcttagttgtcatgccaagggtggtggatatatctttggtggtgccggaactattatagatatcggtggtcaggatataaaggtattgaagctaaatgacaagggtggtcttgttaacttcctgatgaatgataagtgtgctgccggtacaggtaggttcttggaagttatgtctggcgtattggatgtaaagctagatgaactaggggaactagatgccaaggctacagaagttacaccaatcagttctacatgtacagtttttgctgagtcagaagttatatcatgtatggctaagaagattcctctagaaaatatcataagaggtatacacgcatctgttgcaacaagggttgctagtttggcaagaagaggtggtttgaagactcctgtagccatgacaggtggagttagtaagaacaagggtatagtaagggctcttaaagaagaactagaatgtgatatcttgatatctcctgattctcagatggctggtgctataggtgcagccctatatgcatatgacgaataccagaagcaaaacgcttaa

SEQ ID NO: 147 - DNA, 1119 bp, Peptostreptococcus stomatis
atgagtaatatagatgtattgttaggtaaacttgatgtaagtcttttgggacaggtagacaagtatgtttcagaaggtaagaaggtaataggttgcgcgccagtttatacaccagaagaattagtatatgctgcaggcatggtaccaattggtgtatggggtgcagaaggtgaagtaggtctatcaaaggaatacttcccagcattttatgcagctataatccttagattaatggaccttggtttagaaggtaagcttgacaagatgtcaggtatgattataccgggactaagtgacggtctaaagggacttagccagaactggaagagggctataaagcaggttccggccctatacataggctatggtcagaacagaaaaattgaagctggtattacttacaatgaaaagcagtacatcaagctaagaggacagttagaagaaatagctggttgcaagatagaagatgctaaggttgaagaggctatagttctttacaacaagcacagaaaggcaatgcaggaattcagttctctagcagctagtcacttaaatactattacacctattctaagagctagagtaatgacaagtgccttcttgttcgacaaggcagaacatttagctatattggaagaattgaataaagaattaaaggcgttacctgaagaaaaatttgctggcaagaaggtagttactactggtattcttgcaaatagcccaggtatgctagaaatactagatgagtacaaacttggtatagttgatgacaatatcaaccatgaatcaggccagtttgactacctagttgatgaaggtactggtaatccagttagagccttatctaagtggatttcagatatagaaggaagtactttgttgtatgatccagaaaaacttaggggacagataataattgacaaggttaagaagcatcaggcagatggtgttatatacctaatgactaagtttagtgattctgatgaattcgactatccaatcatcagaaaagaattagaaaatgcaggtatcttgcatatactagttgaggttgatcagcaaatgactaactttgaacaggcgaaaacagcattacagactttcgctgatatgatttaa

SEQ ID NO: 148 - DNA, 1236 bp, Peptostreptococcus stomatis
atgagtaatacaggaatggtagaagaaaagccggcaaaagtattgttaggagaaattgttgcaaagcactataaggaagcttgggaggcaaagaataatggtgaactagttggatggtgtgcatctaacttcccacaggaaatattcgaaactatggatataaaggttgtttatccagaaaaccaggctgctgctatatctgctaagggtggcggacagagaatgtgcgaaatagctgaaaatgaaggatattcaaatgatatctgtgcttacgctagaatatctttggcatacatggacgttaaggatgctccagaattaaatatgccacagccagacttcgttgcttgctgtaacaatatttgtaactgtatgatcaagtggtatgaaaatatagctaaggaattgaatatacctttaattttaatagacgttccttacaacaatgactacgaggctgaagacgatagagttgaatatctaagaggtcagtttgattatgctatcaagcagttagaagaactaactggcaagaagtgggatgaaaagaagtttgaagaagtaatggaagtttctcagagaacaggtagggcttggttaaaggctactggatatgctaagtatactccatcaccattctcaggctttgacgtattcaaccacatggctgttgcagtttgtgcaagaggtaagatagaatcagctatagcattcgaaaagctagctgaagaatttgacgaaaacgtaagaactggtaagtcaacatttaagggcgaagaaaagttcagggtgttatttgaaggtatagcatgttggccacacctaagacatacattcaagcagcttaaggatgctggtgttaatgtctgtggtacagtatatgcggatgctttcggatatatctatgacaatacatatcagttaatgcaggcttactgcggaactccaaatgctatttcatacgaaagggcaactgatatgagactaaaggttattgaagaaaacaatatagatggtatgttaatccacatcaacagaagttgtaagcagtggtcaggtatcatgtacgagatggaaagagatattagagaaaagactggtataccaacagctacattcgatggtgaccaggccgatccaagaaacttctctgaagctcagtatgatactagagtacagggtcttatagaactaatggaagctaataaagctgcaaagatgaaggaggcgcactaa

SEQ ID NO: 149 - DNA, 1227 bp, Clostridium difficile
atgtctgaaaaaaaagaagctagagtagtaattaatgatttattagctgaacaatatgcaaatgcatttaaagctaaagaagaaggaagacctgtaggttggtcaacatcagtatttcctcaagagttagcagaagtatttgacttaaacgtattatatccagaaaaccaagcagctggagtagcagctaaaaaaggttctttagaattatgtgaaatagctgaatctaaaggatattctattgacctatgtgcatatgcaagaacaaattttggtcttttagaaaatggtggatgtgaagctttggatatgccagctccagatttcctactttgctgtaacaatatatgtaaccaagttataaaatggtatgaaaatatttcaagagaattagatatacctttaataatgattgatacaactttcaataatgaagacgaagttactcaatcaagaatagattatattaaagctcaatttgaagaagctataaaacaactagaaattatatcaggaaagaaatttgaccctaagaagtttgaagaagtaatgaaaatatcagctgaaaacggaagactatggaagtattctatgagtttaccagcagattcttctccttctccaatgaatggatttgacttatttacttacatggctgtaatagtttgtgctagaggtaaaaaagaaactacagaagcatttaagttacttatagaagaattagaggacaacatgaaaactggtaaatcttctttcagaggggaagaaaaatacagaataatgatggaaggtataccttgttggccatatataggatacaagatgaaaacattagctaaatttggagttaacatgacaggtagtgtttacccacatgcttgggcattacaatatgaagttaatgatttagatggaatggcagtagcatatagtactatgtttaacaatgtaaacctagaccgtatgacaaaatatagagttgattctttagtagagggtaaatgtgatggagcattctatcatatgaacagaagctgtaaacttatgagtttaatacaatatgaaatgcaaagaagagcagctgaagaaactggattaccatatgctggatttgatggtgaccaagcagaccctagagctttcactaatgctcaatttgaaacaagaattcaaggtttagttgaagtaatggaagaaagaaaaaaacttaatagaggtgagatataa

SEQ ID NO: 150 - DNA, 1125 bp, Clostridium difficile
atggaagctattttatctaaaatgaaagaagtagttgaaaatccaaatgcggctgtaaaaaaatataaaagtgaaactggtaaaaaagctataggttgtttcccagtttattgcccagaagaaattatacatgcagctggaatgcttccagttggtatatggggaggacaaacagaattagatttagctaaacaatatttccctgcatttgcatgttcaataatgcaatcatgtttagaatatggattaaaaggtgcttatgatgaattatctggagttattataccaggtatgtgtgatacactaatttgtttaggacaaaactggaaatcagcagtacctcatataaaatatatatcattagtacacccacaaaatagaaaacttgaagctggtgtaaaatacttaatcagtgagtacaaaggcgtaaaaagagaacttgaagaaatttgtggatatgaaatagaagaagcaaaaattcatgaaagtatagaagtttacaatgaacatagaaaaactatgagagactttgttgaagtagcttataaacattctaatactataaaaccatcaataagaagcttagtaattaagagtgggttctttatgagaaaagaagaacatactgagctagtgaaagatttaatagcaaaattaaatgctatgccagaagaagtctgttctggaaagaaagttttattaacaggtatattagctgattctaaagatatattagacattttagaagacaacaatatatcagttgtagctgacgacttagcacaagaaacaagacaattcagaacagatgtaccagcaggtgatgatgcgttagagagattagcaagacaatggtcaaacatagaaggatgttcattagcttatgaccctaagaaaaaacgtgggtcacttatagtagatgaagttaaaaagaaagatatagatggtgttatcttctgtatgatgaaattctgtgacccagaagaatacgattatcctttagttagaaaagatatagaagatagtggaatacctactttatatgttgaaatcgaccaacaaactcagaataatgaacaagccagaactcgtattcaaacttttgctgagatgatgagtttagcg

SEQ ID NO: 151 - DNA, 798 bp, Clostridium difficile
atgtacacaatgggattagatataggttcaactgcatcaaagggagtaatcttaaagaatggggaagatattgtagcttctgaaacaatatcctctggtactgggactactggaccatcaagagttttagaaaaattatatggcaagacaggtcttgcaagagaagatattaaaaaagttgtagttacaggatatggaagaatgaactattcagatgctgataagcaaataagtgaattaagctgtcatgctagaggggtaaatttcataattccagagacaagaaccattattgacataggtggtcaagatgcaaaggtattaaaattagataataatggaagactattaaactttcttatgaatgacaaatgtgctgcaggtacaggaagatttttagatgtaatggcaaaaataatagaggttgatgtatctgaactcggaagtatatctatgaattctcaaaatgaagtatcaataagcagtacatgtacagtatttgcagagtctgaggttatatcacatttatctgaaaatgcaaaaattgaagatatagtggcaggtattcatacttcagtagcaaagagagtttctagcctagtaaaaagaataggagtacaaagaaatgtagttatggttggtggggttgctagaaatagtggtattgtaagagctatggcaagagaaatcaacacagaaattattgtacctgatatacctcaattaactggtgctttaggagcagcgttatatgcttttgatgaagcaaaagaatcacaaaaagaagtgaaaaatata

SEQ ID NO: 152 - DNA, 1194 bp, Clostridium difficile
cttttagaaggagttaaagtagtagaactttcaagtttcatcgcagcaccatgttgtgcaaaaatgttaggtgactggggtgcagaggttattaagattgaacctatagaaggtgatggaataagagttatgggtggaacatttaaatctccagcatcagatgatgaaaaccctatgtttgaattagaaaatggaaataaaaagggtgtaagtattaatgtaaaatcaaaagaaggagtagaaatattacataaattattatcagaagcagacatatttgtaactaatgttagagttcaagcattagaaaaaatgggtatagcttatgaccaaataaaagataagtatccaggattaatattctctcaaatattaggatatggtgaaaaaggacctttaaaagataaaccaggatttgactatactgcatacttcgcaagaggaggagttagccaatctgttatggaaaaaggaacatctccagcaaatacagcagcaggatttggtgaccactatgcaggtctagcactagcagcaggaagtttagcagcattacataaaaaagctcaaactggtaaaggtgagagagtaacagtaagtcttttccatacagctatatatggaatgggaacaatgataacaacagcacaatacggaaatgaaatgcctttatcaagagaaaatccaaacagcccattaatgactacatataaatgtaaagatggaagatggattcaattagctttaatacaatacaacaagtggttaggcaaattctgtaaggttataaatagagaatatatattagaagacgatagatataataacatagattcaatggttaatcatgttgaagatttagttaagatagttggagaagctatgttagaaaaaacattagacgagtggtcagctttattagaagaagcagacttaccatttgaaaaaattcaaagctgtgaagatttattagatgacgaacaagcttgggcaaatgacttcttatttaagaaaacatacgatagcggaaatacaggtgtcttagttaatactccagttatgtttagaaatgaaggaattaaagaatatacaccagcaccaaaagtaggtcaacatactgtagaagtattaaaatctttaggctacgatgaagagaaaataaataactttaaagatagtaaagttgtaagatat

SEQ ID NO: 153 - DNA, 768 bp, Escherichia coli
  MISC_FEATURE (1)..(768): strain K12
atgagcgaactgatcgtcagccgtcagcaacaagtattgttgctgacccttaaccgtcccgccgcacgtaatgcgctaaataatgcccttctgacgcaactggtaaatgaactggaagctgcggctaccgatagcagcatttcggtctgtgtgattaccggtaatgcacgcttttttgccgctggggccgatctcaacgaaatggcagaaaaagatctcgcggccaccttaaacgatacacgcccgcagctatgggcgcgattgcaggccttcaacaaacctctcatcgcagccgtcaacggttacgcgcttggtgcgggttgcgaactggcattgttgtgcgatgtggtggttgccggagagaacgcgcgttttggtttgccggaaatcactctcggcatcatgccaggcgcaggaggaacgcaacgtttaatccgtagtgtcggtaaatcgttagccagcaaaatggtgctgagcggagaaagtatcaccgctcagcaagcacagcaggccgggctggttagcgacgtcttccccagcgatttaaccctcgaatacgccttacagctggcatcgaaaatggcacgtcactcgccgctggccttacaagcggcaaagcaagcgctgcgccagtcgcaggaagtggctttgcaagccggacttgcccaggagcgacagttattcaccttgctggcggcaacagaagatcgtcatgaaggcatctccgctttcttacaaaaacgcacgcccgactttaaaggacgctaa

SEQ ID NO: 154 - DNA, 774 bp, Rhodobacter capsulatus
atgagctatcacacgatccgctacgagatctccgaagggctggcggtgatcacgctcgatcgccccgaggtgatgaatgcgctgaacgcggcgatgcggcacgaattgaccgcggcgctgcaccgcgcgcggggcgaggcgcgggcgatcgtgctgaccggatcggggcgggccttttgctctgggcaggatctgggcgatggcgcggccgaggggctgaacctggaaaccgtgctgcgcgaggaatacgagccgcttttgcaggcgatttacagctgtccgctgccggttctggcggcggtgaacggcgcggcggcgggggcgggggccaatctggctctggcggccgatgtggtgatcgcggcgcaatctgcggccttcatgcaggctttcacccggatcgggctgatgccggatgcgggcgggacctggtggctgccgcggcaggtcggcatggcccgcgccatggggatggccctgttcgccgagaagatcggcgccgaagaggccgcgcgcatggggctgatctgggaagccgtgcccgatgtcgatttcgagcatcactggcgggcccgggcggcgcatctggcgcggggcccttcggcggcctttgcggcggtgaagaaggcctttcatgccggtctgagcaatcccctgcccgcgcagctggcgctggaagcccggttgcagggcgaactgggccagagcgcggatttccgcgagggcgtgcaggcctttctggaaaagcgcccgccgcatttcaccgggcgctag

SEQ ID NO: 155 - DNA, 2106 bp, Pseudomonas stutzeri
atgacggatgtcattcggctcgaacgccggggcgatatcgctctgatcctggtcaacaacccgccggtcaacgcccttggccatgccgtacgaaaaggcctgttggatgcctttcaagaggctgacgaggcgcccgaggtgacggccgtggtgctggtctgcgaaggcccgaccttcatggccggcgccgatatcaaggagttcggcaaaccgccgcaggcaccgagcctgccggaggtgatcgaggtgatcgagggctgccgcaagccgagcgtcgcggtgatccacggcaccgccctgggtggtgggctggaggtcgcgctgggctgccattaccgtatcgcccggtcggacgccaaggtcggcctgccggaggtgaagctgggcctgctgcccggcgccggcggtacccagcgcttgccgcggctggccggtgtcgagaaggcgctggagatgatcgtcagcggccagcccatcggtgcggcggaggcgctggagcactatatcgtcgacgagctgttcgaaggcgatctgatcgaggccggtctgacctatgcgcgtcgccttgtcgaggagggccgcggtccgcgccgcagtggcgagcagacccgcggtctggaaggcgtcgacaacgaggcgctgattcgcgccaagcacgccgaggtggccaagcgcatgccggggctgttctcgccgctgcgctgcattgccgcggtggaagccgccaccaggctgccgctggccgaaggcctcaagcgcgagcgcgagttgttcaccgagtgcctgaattcaccgcagcgcggcgcgctgatccattcgttcttcgccgagcgtcaggccggcaagatcgacgacctaccatccgacgtcaccccccgcccgatcaggaccgccgcggtgatcggcggcggcaccatgggcgtcggcatcgccttgagcttcgccaacgccggggtgccggtgaagctgctggaaatcaatgacgaggcgttgcaacgcggcctgcagcgtgcccgcgaaacctacgcggcgagcgtcaagcgcggcagcctgaccgaggatgcgatggagcagcgcctcgcgctgatcgctggcgtcaccgactacggcgccctggctgatgccgacgtggtggtcgaggccgtgttcgaagagatgggcgtcaagcagcaggtcttcgagcaactggatgcggtgtgcaagccgggtgcgatcctcgcctccaacacctcgtcgctggacctgaacgccatcgccggcttcaccaggcgccccgaggatgtggtcggcatgcacttcttcagcccggccaatgtcatgcgcctgctggaagtggtgcgcggtgagcggaccagcgatgaagtgctcgccgccgccatggcgatcggcaagcagctgaagaaggtctcggtggtggtcggcgtctgcgacggcttcgtcggcaaccgcatggtcttccagtacggccgcgaggcggagttcctgctggaggaaggcgccacgccacaacaggtcgacgctgccctgcgcaatttcggcatggccatgggaccgttcgccatgcgcgatctgtccggtctcgacatcggccaggcgatccgcaagcgccagcgcgcgacgctgccggcgcacctggattttcccaccgtctcggacaagctctgcgccgccggcatgctggggcagaagaccggtgccggctactaccgctacgaacccggcaaccgcaccccgcaggagaatcccgacctcgcgcccatgctggaagccgcgtcgcgggaaaagggcatcgagcggcaggcgctggacgagcagtacatcgtcgagcgctgcatcttcgcgctggtcaacgagggcgcgaagattctcgaggaaggcattgcccagcgctccagcgacatcgacgtcatctacctcaacggctacggcttcccggccttccgcggcgggccgatgtactacgccgacagcgtcggcctggacaaggtgctggcgcgagtaaaagaactgcacgcgcgttgcggcgactggtggaagccggcgccactgctggaaaaactggccgccgaaggccgcaccttcaccgaatggcaggccgggcaatga

SEQ ID NO: 156 - DNA, 1968 bp, Haliangium ochraceum
atgatcgtcggagtcatcgggtcgggcgccatcggcccagacctcgcctacggattcgcctcggccctggccagcgttcccggcgccagggtctatctacacgatatcaagcaggaggccctcgacgccggtatgcagcgcatccgcggctacatcgccaagggcctggcccgcggcaagatcagcgaacgcgtcgccggcgccctggagacggtgctcgtgcccacgctctcgctcgccgatctcgcgccgtgcagctacgtgctcgaggccgccaccgaggagctcggggtcaagcgcgccatcttgcgcagcctcgaggatacagtcgatagcgagtgcctcatcggcttcgccacctcgggcctgccgcgcgcgatcatcgccgccgaggtcaaacatcccgagcgctgcttcgtcaatcaccccttctaccccgcctggcgttcgctgcccgtcgaggtcgtgctctcgggtagcccggcgcacggccagcgcatgctggccaccctcgaggccctgggcaaagtccccgtcatcaccgcggacgcgccctgcttcgcggccgacgacatcttttgcaactactgctcggaggccgcgcgcatcgtcgaggaaggcatcgccaatcccgcccaggtcgacgccatcgtccacggcgccatcggcggcggcggcccgctcaacgtcctcgacgccacccgcggcaacctgctcaccgtgcactgccaggagctgatgcgcgacgccgacaccggcacgccgtggttcgagccgcccgccatcctgcgcgagcgcggcgacgccctgtggcacgatcccaaggccccgcacgaccccgccttcgacgaggccctgcgcgagcgcgtgctcgaccgcatcctggccgtgctgctcgcgcgcacagtgttcgtgctcgatcacggcatctgcgccgccaccgagctcgactggatgacgcgcaccgcgctcggcttccgcaccggcttggtcgacctggtggacgaactcggccccgagcgcgtggccgagctgtgccagcgctacgccgccgagcaccccggcttcgtcatcccggacagcatccgcgagcagcacaagccgcgcttctacggcaacctgcgcgtcacccgccaggacgagctggccatcgtgcgcatcttccgccccgaggtgaagaacgcgctcgaccgccgcaccctgagcgagctcgaccacctcatggccgcgctgtcggccgacgacagcgtcgagggcgtggtcctgagcagcgccggcggcgcgctggccggcgccgacatcaccgagctagcgcgcgtgcgcaccaccgaggaggcggtgtccacctgcgctttcggacaagcggtcttgaaccgcatcgcggccatggacaagcccgtggtcgccgccgtcgacggcccggtgctgggcggcggcgccgagctgtcgatggcgtgccatgcgcgcgtcgtcggcccgcgcctgagcatgggccaacccgaggtcaacctcggcatcatccccggctacggcggcacccagcggctgccgcggctcatcggcgtggagcgcgcgctggccatgatgcgcacggcgcagagcatcgacgcgcagaccgcgtgcgagtggggctgggccagcggcacgccgatggtcgacttcgtcggcgcggccgcgaccctcatccgcagccacctcgccggcgaggccgagctcgcgccgctcgaccccgcgcccatgagcgtacccgccgcggccgcccccgtggacatcggccaccgctcgcgcgtcatcgacgagatcctcgtggatgtggtccagtccggcttgcgcgcgccgctgagcgagggcctggccaccgaggccgccggcttcggccgctgcgtgctcaccgtggacctcgacatcggactcaagaacttcatgcagaacggcccccgggttccggcgctgttcctccacgagtag

SEQ ID NO: 157 - DNA, 768 bp, Anoxybacillus flavithermus
atgttttctattcaacaagaggggtatgtggcgattttagcacttcatcgtccaccagcaaacgctttagcatcttctgttttgaaagagctttcagaacggcttgatgcattaaaagaagacgaacaagtacgtgtcatcgttcttcacggagaaggaagatttttctcagctggtgccgatattaaagagtttacagcgatcgaggcgagcgaacaagcggctgaacttgctcgagctggacaacaagtgatggagaaaattgaacagtttccgaaaccgattattgccgcgattcacggtgctgcacttggcggagggctcgagttagctatgagttgccatctgcgcatcgtagcggaaaacgccaaacttggcttaccagaattgcagctcggcatcattccgggatttgcaggaacacaacgcttattgcgtcatgtcggtatggcaaaagcgctagaaatgatgtggacaagcgaaccgatcacaggtgcagaagctgtgcagtggggactagcaaacaaagccgtcccagaagaacaattgcttgatacagcgaagcaacttgcacaaaaaattgctcaaaagagcccgatttctgttcaagcggtattgaaactagttaatgaagctcgcacaaaaacgttccatgaatgcgttgaaaaagaggctcaactgtttggacaagtctttgtaacagaagatgcgaaagagggcatttcggcatttatcgaaaaacggacaccacagtttcaaggaaaataa

SEQ ID NO: 158 - DNA, 783 bp, Streptomyces avermitilis
atgagcacggcgcccgaagctgccgacttggtgctccacgagcgtcacggcggcgtactgaccatcaccatcaaccgccccgcgcagaagaacgccgtcgaccacgaggccgcggtacagctcgcggcggccgtggatctgctcgacgcggacccggagctgtcggtcggcgtcctcacgggcgcgggcggggtgttcagcgcgggcatggacctgaaggcgttcgccaagggcgagctgcccttgctgcccagccggggcctgggcgggctcacccgcgcgtcggtgcgaaagccgctggtcgccgcggtcgagggctgggcgctcggcggtggcttcgagctggtcctcgcctgcgacctgatcgtcgccgcggaggacgcccgcttcgggtttcccgaggtcatgcgtggtctcgtggcggcggagggcggactggtcaggctgccgcgccgacttccgtaccacgtcgccgcgcgcgtactgctgacgggcgagccgctgaccgccgtcgaagccaaggagtacgggctcgtcaatgagctgaccccgcccggcgccgcgctggacgcggcccgggagctcgcgggccgcgtcgcgcggaacgcaccgcttgcactggcggccgtcaaggaggtcctgcgcgagacacagggcctgaaggagagcgacgcgttcagacgccaggacgagctcacgagcggactggccgccagcgaggacgcgcgggaaggcgcacaggcgttcgccgagaaacgcgccccggtctggcacggccgctga

SEQ ID NO: 159 - DNA, 1683 bp, Advenella kashmirensis
gtggacaatggccgtaagctgattgaacgtggctggcatttattcaaccgtatcgaaaagctagcctttcctacactggcactcatgcacggcccctgcctgggtggcgggctggaactggcactggcgtgccgttatcgaatcgcgatcgattctcccaagccggtgatcggcctgcctgaagtcaaattgggcatcttccccgcctggggcggcctgatgcgactaccccgcctgattggtccgcaaaccgccctgaacatgatgctgaccggtcgcacactggatggccgcaaggccaggtctgccggtctggtagatttgctggtcgcaccccgagttgcagagaaatcggcgatcgatctggtcacgtcgggcaaaccggcgcgtcaggctcgcggcctggccggcttgctcaatcgtgcaccgttcaagtcgctggtggctgcccaggcacgcaaaagcgtcaagcaaaaagacccttatggccactaccccgccaccctgaccatgctggatctgtgggaaaaacatgatggcgacccgttggccgatccccaggcgctgacccggctgctgcaatcggatgtcacccgcaatctgatccgtgtatttcacctgcaggagcggctcaaggcgtttggcaagaaggataatgccactcccgtcaaccatgttcatgtgatcggggccggcgtgatgggcggtggcatcgctgcctggtgcgcgctgcagggcatcaaaaccaccttgcaggataccgacgcccagcgcatcgccggggcgttcaaaaacgccgtctccatttatgcccgcaaggatcggtataccgcgcaggcagcccgcgatcgcctgattccggacctggcgggccacggtatcgcgacggctgatctggtgattgaagcgatcagcgaaaatccgcaagccaagcaatcgctctaccagcagatcgaaccaaaaatgaaagaaggcgccattttagccaccaatacatccagtctgtccattgcgcagttacgcagcgtgctggtgcaccccgaacgttttgtcggtattcattttttcaatccagtctcacgcatgccgctggtagaagtggtacatgccgatggcatcgcccaggaaactctggacaccgctgccgcctttgtcggcaaaatcggcaaactgccgctgccggttcaggacacgccgggctttctggtcaacgccgtgcttgctccctatatgctgcaagccatgcggtgcattgacgaaggcatggatcccgaagtcatcgataccgcaatgctggagttcggcatgcccatggggccgatcacgctggccgatacggttggtctggatattgccatggcagccggcaaacagctgtccgaaggccaggagccgccacgctgcctgcaagagaagattgcccaaggcaagctgggtgtcaaaagcggcgaaggcttttacgtgtggaaagaccgcaagcatgaccagcgcagtagcaaagccatcccgcaaggcctggcacagcgcctgatcaagccgctgatagagcagaccgaaaaacaacttgcgaacaacatcgtgcaagatgcagatcttgccgatgcaggcgtgatattcggaaccgggtttgcgccttttaccggaggacccattcattacaaacaaagtaaaggaggactatga

SEQ ID NO: 160 - DNA, 714 bp, Oligotropha carboxidovorans
gtgagcctttcgccgcttgccaacggcgtacgcgttctcacactggatcgtccgtccaaggccaacgcgttgaatgcggaggtcgtggaccagttgcttgcgtgtgtcgcccaggccgaggcggaggattgccgcgtgctgatcctcgccgccaacggcaaggcgttttgcggcgggtttgatttcggtggttatgaatcgatgtcggcgggcgacctgctgctgcgctttgtccggatcgaggagttgctgcagcggatgcgccagtcgtcgtttgtcagcattgctctggtgcatggtgcggcgatgggggcgggggcggacatcgtcgcgtcttgcacctatcgcatcggcaccgacgcaagccggtttcgctttccgggattccgtttcggcgtggcgcttggcacgcggcatctggcgcagcttgtcggcccgcaacgggcgcgcgatatcctgctgaccaatgcaacgatcgatgcattgaccgctgtcgatatcggattgctgacgcacctcgtcgatgccgggagcatgcggcagaaagcggacgagattattgcgcagattggctcgctggaccgtgtcgcacgcaaccggattttgcatctgacctcggctcagaacaatgacggtgacatggctgagctggtgaaatcggtgagcgcgcccgggctacacgagcgcattgcgcagtaccgcgccgggcattga

SEQ ID NO: 161 - DNA, 801 bp, Riemerella anatipestifer
atgtacaaattaatagatgtagataaccattttgaaggaaagcttcaaatcgcatatatcaatcagccagaatcgtttaatagtcttaataaggttgttttagaagagttattgcactttataaaagcttgtgacgcagattctagtgtacgctgtattgcaattagtggcaaaggtaaggcgttttgttctggtcagaatttaaaggaggctttagattataaagcagaagccaatgaggaacgctttatccaaaggattgtgatagattattataatccgttagtgaaggctattgtctatgctaaaaaaccagtaattgcattggttaatggtcctgcggttggtgcaggagcaatgttagctctcatctgtgattttgcagtggcgtcagagtcagcgtatttttccttagctttttctaatataggactagtgccagatacggcaggtacttactatttgcctaaacttttagggcgttccttagcgagttatttggcatttacagggaagaagctatctgctaaagagtctttagaaagaggtttggtggtagatgttttttcagatgctactttttcggaacaatctttacaagtcctagaacatattactcatcagcctactgtggcattggggcttacaaaaaaagcctttaataaatcttatcagaatagtctatcggagcagttagatttggagagtattctccagcaagatgctgcagaaacttgggattttcaagaggggatagccgcttttttagcaaaaagaaaacctcagtataaaggtaagtaa

SEQ ID NO: 162 - DNA, 1269 bp, Fusobacterium necrophorum
  MISC_FEATURE (1)..(1269): subsp. funduliforme Fnf 1007
atgtcagaaacaatcaatttagatgaaatgtcagcaaaacaattattgggttattatcaagaaaaattggatgaagaagcaagacaggcaaaaagagaaggaaaattagtttgttggtctgcttccgttgctccaccagaattctgtgtagctatggatattgccatggtgtatccagaaactcatgcagcagggattggagctagaaaagggtcgttagatctgctagaagtagcagatgaaaaagggtattctttagatatttgttcttatgcaagagtaaatttggggtatatggaattgttaaaacaacaagccttaactggagaaactcctgaaaaattagcaaactctccggctgcaaaagttcctttaccggatttagttattacatgtaataacatttgtaatactttgttaaaatggtacgaaaatttggcaaaggaattaaatattccatgtattgtaattgacgttccgttcaatcatactatgccaattacaaaacattcaaaagaatatattgcagatcaatttaaatatgcaattcaacaattagaagaaattacaggaaagaaatttgactatgataaattcttagaagtgcaagagcaaacacaaagatctgtatatcaatggaatcgtttagcagctcttgctcactacaaaccttctccattaaatggtttcgatttatttaacttcatggctttaattgtatgtgctagaagtagagattatgcagaaatcactttcaagaaatttgcagatgaattggaagaaaacttgaaaaatgaagtatatgcgttcaaaggagctgaaaagaacagagttacttgggaaggaattgcagtatggccttaccttggacacactttcaagtctttaaaaggaatgggaagtatcatgaccggttctgcatatccaggaatctggaacttgacatatactcctggagatatggaatctatggcggaagcatatacaagagtctacattaatacttgcttacaaaataaagcggatgtcctttctaaaattgtaacagacggaaaatgtgatggaatactatatcatttgaatagaagttgtaaactgatgagtttcttgaatgtggaaactgctgaattagttgaaaaagcgactggagtgccatatgtaagtttcgatggagaccaaacagatccgagaaatttcgcaccggctcaatttgatacaagagtacaagctttaaatgaaatgatggaagttaataacgaaacaaaataa

SEQ ID NO: 163 - DNA, 834 bp, Fusobacterium necrophorum
  MISC_FEATURE (1)..(834): subsp. funduliforme Fnf 1007
gtgcaagatgacagaagttttaagaaaggaaagagaagaggaatgtatacagttggagtggatataggttcttcttcttcaaaagtagtgatattaaaggatggaacagagattgtaagtcaatcggcaattcagtcgggaattggaagtaatcgagccattgttgctttggaagataatttaaaaaaagcaaacttgacgaaggaagatattggttttacagttgttactggatatggacgctttacttttgaaggagcagataaacaaatcagcgagattagttgtcatgccagggggattcattttttattaccgaatgtgagaaccattattgatattggtggacaagatgccaaagcgatcagcttagatgaaaaaggtcatgtaagacaattttttatgaatgacaaatgtgcagcaggaacaggacgatttttaactgtaatggcacgcgtactagagatttccctagatgagatgggaacttatgatgctctttctaaaaatccttgtaatattagtagtacttgtgctgtatttgcagaatcagaagtcatttctcaattggcaaagggaaataccaaagaggatgtcattgcaggagtacataattctgtcgctcataagatattaggtttagtatatcgtacttctatggaagaaaaatttgcgatttgtggtggtgttgctcagaatacaggtgcattgcgtgcaatacgggaagctttgaaaaaagaagtaatcgttgctcctaatccacaattaacaggagcattaggagctgcaatttttgcttatgatgagctgaaaaaattaagaaagggtgaataa

SEQ ID NO: 164 - DNA, 1125 bp, Fusobacterium necrophorum
  MISC_FEATURE (1)..(1125): subsp. funduliforme Fnf 1007
atgaaaggcagattagaagaattaattcatatatttgaagatgttgcaaacaaccccaaaaaaatggtagcagaatataaaaaagaagtagggaaagaagtgattggagtcatgccagtatatgctccagaagaaattattcacgctgctggatgtttacctattggattatggggaggaaaaaaagaagtttctaaagcaagagcatatttacctccttttgcatgttctattatgcaaactgttatggaattacaaattggaggaacatatgacattttagatgcagtattattctctgtaccttgtgatactttgaaatgtttaagtcaaaaatggaaaggaaaatctcctgtaattgtatttactcatcctcaaaacagagtaattgaaggagcaaatgcttacttagtaaaggaatatcaagcagtaaaagaaaaattagaaggaatcttaggaagaaccattcctatggaagcgattgaagaaagcgtaaaagtatataatgaaaatagaagagttatgagagaatttgtagaagtggcggcacaatatccacaaattatcgatccaattgttagacataatgtgatgaaatccagatggttcttaagaaaagaaaaacatactgaatatgtaaaagaattaatcgctgaattaaaaaaagaaactattgttccttgggacggaaagaaagtaatcttaacaggaattatgacagaaccagtagaattgttgcaaatctttaaagatgaaaaacttgctattgtagccgatgatttagctcatgaaagccgacaatttagaggagatgttcctgaagaaggaggagatgttctatacagaatggcaaaatggtggcaaaatttagaaggatgttctttagcaacggatactaataaaggtagaggacaaatgctaatggatatgtgtaaggatacgaaagcagatgccgttatcgtgtgtatgatgaaattctgtgatcctgaagaatttgactatccggtatactatagagaatttactgaatccggaattaaaaatattacagtggaagtggacttagaagtttcttcttttgaacaaattagaacaagaatacaaacatttaaagatattttataa

SEQ ID NO: 165 - DNA, 1269 bp, Desulfosporosinus youngiae
  MISC_FEATURE (1)..(1269): DSM 17734
atgacggatacaacaactatgagtgccaaagaattgttaggtttctatcaggaagaattgtatgaagaagcgagacaggccaaaaaagaaggaaaacttgtttgttggtctgcatcggttgctccttcggagttttgtgtggctatggatgtggcgatgatctatcctgaaacacatgctgcggggattggggcaagaaaaggtgccttagatgtgctggaagttgccgatgaaaaaggctataacctggatacttgctcctatgcaagagtcaatatgggttatatggaacttctgaaacaagaggctttaacaggaataacgccggaaaagcttgaaaaatccccggcggccagaataccgctgcccgattttgtcataacctgcaacaacatttgcaacaccttgcttaagtggtatgagaatcttgccgttgaattaaatattccctgcatcatcattgatgttccctttaatcataccatgcccattccccagtatgctaaggactatattgcggaacagtttaaggaggctattactcagcttgaggaaatttgcggcaggaaattcgactacgacaaatttttgaaagtacaggaacaaacccagcgttctgtggcccagtggaacagaattgctgctttgtcgggacataaaccatctcctttaaatggttttgatcttttcaactatatggccctgatcgtttgtgccagaagcagagactacgcggaaattacctttaaaaagtttgccgatgaacttgaagaaaacctcaaaaacggtatctacgcctttaaaggaaatgaacaaaagcgtgtaacttgggagggcatagctgtttggccgcatctgggccatacatttaaaggcttaaagaatctgggcaatatcatgacaggttcggcttatcccggtttgtggaatcttacctacacacctggggatatgagttccatggcggaagcttataccagaatttatatcaatacttgtctcgataacaaagttaaggtgcttagtgacgtcatcagcggcggaaagtgtgacggggttatttatcatcagaacagaagctgtaagctcatgagtcttctcaatgtcgaaacggctgatatactccaaaaacaaaatcatttaccctatgtcagctttgatggggaccaaacggatcctcgtaactttgctcctgcccagtttgatacacgtatccaggccttagatgaaatgatgaagcagaataaggagggagtttccaatgagtag

SEQ ID NO: 166 - DNA, 1119 bp, Desulfosporosinus youngiae
  MISC_FEATURE (1)..(1119): DSM 17734
atgagtagaattgaaacgattatcagtgaattaacgtccattgccaataatccccgccaggctatggaagattataaaaaagaaaccggcaaagggtcggttggggttatgccttattatgctcctgaagaaatcattcatgccgcagggtatctgcccgtaggtatttggggaggacaaaagagtatttccaaggcccgggcctatttgcctccctttgcttgttcaattatgcaatccgtggtggaaatgcagcttgaaggggtctatgacgatttagaagcggtccttttccctgttccttgtgacaccttaaaatgtcttagccaaaaatggaaaggaacctcccctgtcatcgttttaactcatcctcaaaacagaaaactggaagcagccaataagtttcttgctgaggaatataggcttgtgcgtgaaaaactggaaaaaatcctgaatgttaagattacagacgaggcacttaaccaaagcattgaaatttataacgaaaatcgtaaagtaatgcgtgaatttacagagatagctgctaattatcccaacattattgatcccgtaaaacgtcatgcgcttatcaaagccagattctttatggaaaaagccaaacataccgctctggtcaaagaattgaatgcagagcttaaagcgttaccggtggaagcctttacaggcaaaaaggttgttttgacaggcattatggctgaacccaatgaagtattggacattttgcaagataacggttttgctgttgtggcagatgacctggcccaggaatccagactgttcagaaatgatgttccctcagggacagacccactctatcgcttggctaaatggtggcaggaattcgatggttgttctctggctgtcgatgcgaaaaaaccaagaggccccatgctgatggatatggttaaagcatctaaggccgatgccgttgtggtttgcatgatgaagttctgtgaccctgaagaatttgactatccaatctactacagacagtttgaagaagccggaattaagagcttatttatagaaattgacctggaaccaacctcctttgaacagactaaaaccagagttcaaagttttagagaaatgctgtga

SEQ ID NO: 167 - DNA, 819 bp, Desulfosporosinus youngiae
  MISC_FEATURE (1)..(819): DSM 17734
atgtttacaatggggattgatattgggtcctcatcctcaaaggttgtaatccttgaagatggagttaatattatcgccggagaagttattcagattggaacaggttctacgggacctaaacgtgtactggatgaagctcttgccaaagcaggtcttacattgcaagacatggctaaaattattgctacaggctatggaagatcgtctgtggaagaagcacacaaacaaatcagcgaaatcagttgtcaggctaagggagttttctttttagttccttcagcaaaattaattattgatattggcggtcaggatgttaaggccattaaacttgacagtaaaggctgtgttaagcagttttttatgaatgataaatgtgccgccggaacaggacgttttctcgatgttatgtcgcgggtactggaagttaatcttgatgaaatggcggaatacgatgcccgggcaacagaacctgccacggtcagcagcacttgcacagtttttgcagaatctgaggtaatatctcagcttgccaacggagttgctaaagagaacattattgcaggggttcaccagtcagttgctagcaaagcctgtggacttgcctatcgatgtggggtggaagaggacatcgtgatgtgcggaggcgttgctaaggacttaggggttgtcagagcaatcagcaaagaactgaaaaaaccggtcattgtagctcctaatccacaaattacagctgcacttggagctgctatatttgccttcgaagaagttatggaaactgttatggttgccttcgaagaagttaggggagctaataaataa

SEQ ID NO: 168 - DNA, 1269 bp, Peptoniphilus indolicus
  MISC_FEATURE (1)..(1269): ATCC 29427
atgaatactatagatatatcaaatatgaaagctaaagaaatgcttggatattttcaaaacaaacttgacgaagaagcacgtgaagctaaaaaaaatggaaaattagtttgctggtcagcctctgtagctccatctgaattttgtgtaaccatggatatcgcattagtttatccagaaactcacgcagccggtataggtgctagaaaaggctctttagctatgttagatgttgctgatagaaaaggttataatacagatatatgttcttatgccagagtaaacttaggatatatggaacttttaaaagaatatgctaagacaggagtgaaacctaaagaacttgaagaatctcctgctgcagatgttcctctacctgatttagtaataacttgcaataatatatgcaacactttactaaaatggtatgaaaatttagctgcagaattaaatattccttgtatagttatagacgttccttttaatcatactatgcctattcctaagtattctaaagaatatattgctgaccaatttaaggaagcaataagacaacttgaagaaataacaggaaaagattttgactatgataaatttttagaagttcaagagcaaacgcaaagatctgttgctcaatggaatagacttgctgcactttctaaatatgaaccgtctcctctaaatggatttgatttatttaactatatggctcttatagtttgtgcaagaagtaaaaattatgctgaattaacttttaaaaaatttgccgatgaacttgaagaaaatatgcaaaatggagtgtatccttacaaggctggagaacaatccagaattacttgggaaggtatagctatttggccatatttaggacacacttttaagactcttaaaggctatggctcaataatgacaggctctgcttatcctggactttggaacttagaatacacacctggagatatgctttcaatggcagaagcttatacgagaatatatataaacacttgccttgacaataaagttgatgtattgagaaaaatcattaaaaacggtaaatgtgatggggtcgcataccatctaaatagaagttgtaaattgatgagtcttctaaacgttgagacagctgaaattttaaataaagaaaataatcttccatatgttagttttgatggtgatcaaactgatcctagaaatttctcagaagcacaatatgataacagaatacaaactcttactgagatgatgtctgccaataaaaaaatgaggggttga

SEQ ID NO: 169 - DNA, 792 bp, Peptoniphilus indolicus
  MISC_FEATURE (1)..(792): ATCC 29427
atgtacactatgggagtagatatcggttctacatcatctaaaatcataatacttgaagatggaataaaaattatcggaaatattgtagtacaatctggaaccggtacaagtgggccaacaattgctactgcaaaagctaagtcctttctttcaaataataatttaactttagatgatatatctaaaatcgttgtcacaggttacggcagattttcatttgatattgccgataaacaaataagtgaaataacttgtcatacaaaaggtattaactttttagtgcctgaagctcgaactattttagatataggtggacaagatacaaaagctatttcagttaatgataaaggtcaagttctacaatttttcatgaatgacaaatgtgccgccggcactggcagatttttagaagtcatggctaaaattttagaaatacctttagaaaaaatgggtgaatatgatagattatcaactaatccggtagctataagtagtacttgtaccgtttttgctgagtctgaagttatttctcagctatcaaagggcatatctaaagaaaatatattagccggtgtacataattcaactgctaacaaagtttgtggtcttttatatcgtacaggaattaaggaaaaaatagttttatgtggaggagttgctcaaaaccaaggtgttgttagagcgctccaagaggaattaaaaaaagaaataaccatagctcctcacccacaaatgacaggcgccataggtgctgctttatttgcttatgaagaggcgaataaaaatttatag

SEQ ID NO: 170 - DNA, 1119 bp, Peptoniphilus indolicus
  MISC_FEATURE (1)..(1119): ATCC 29427
atgaacaaaattaatgaaataataaatttattggatgaagtttctaaagatcctaaactaacagttaaaaaatataaagaaaaaacaggaaaaggtgttgtaggtgtcatgccattatatgcacctgaagaaattattcatgctgcaggttttctacctatgggactttggggtgcacaaaaagaagtatctaaagcaagaatttatttacctccttttgcatgttcaataatgcaaactaatatggaacttcaaatagaaggtgcctatgatgacttagatgcagttgtattttctgtaccgtgcgatactctaaaatgtatgagtcaaaaatggaagggtaaaagtcctgttatagtatttactcatcctcaaaacagaaaattagaatctgcaaataaatttttggttacagaatatgaaatcttaaaagataaattagaaaagatattaaatgtaaaaatatctgatgaatccataacaaatagtattgaaatttacaatgaaaatagaaaagtcatgagagaattttcagacctagctggtcaatatcctaatataattgaccctattcaaagacatattgtatttaagtccagatggtttatggaaaaatcagaacatactaaattagttaaagaactaatatctgaaattaaaaaattacctattgaagaatgggatggctataaagttatagcaactggtattatgatagaacctgaagaaatacttcaaatatttaaagataagaaaatagctattgttgcagatgatttagctcaagaatcaagacaatttagacatgacgtacctgaaggagatcaacctcttttaagacttgctaagtggtggcaaaatttagaaggatgtgctcttgcaactgatacaaaaaaattaagaggccaaatgctaattgatatggcgaaaaaatataatgccgatgctgtattgatatgtatgatgaaattctgcgatcctgaagaatttgactaccctgtatactatagagagttccaagaagctggcataaagaatttactaattgaaattgacttagaaatgacagcttttgaacaaactaacacaagacttcaaactcttgtagaaactctctaa

SEQ ID NO: 171 - DNA, 1269 bp, Desulfosporosinus meridiei
  MISC_FEATURE (1)..(1269): strain ATCC BAA-275/DSM 3257/ NCIMB 13706/S10
atgactgatacaacagctatgagcgccaaagaattgttaggtttctatcaggaagaattgtatgaagaagcgagacgggcaaaaaaagaaggaaaacttgtttgttggtctgcatccgttgctccttcggagttttgtgtggctatggatgtagctatgatatatcctgaaacccatgctgcgggtattggggccagaaaaggtgccttagatgtgcttgaagttgcggatgaaaaaggctataacgtggatacttgctcctatgcaagagtaaatcttggttatatggaacttttaaaacaggaggctttaacaggaataacaccggaaaagcttgaaaaatccccagcggccagaataccccttcccgattttgtcataacctgtaacaacatttgtaacaccttgcttaagtggtatgagaatcttgccgttgaattaaatattccttgcatcatcattgatgttccctttaatcatacaatgcccattccacagtatgccaaggattatattgcggaacagtttaaggaagctattactcagcttgaggaaatttgcggcaagaaattcgactatgacaaatttttaaaagtacaggaacaaacccaacgttctgttgcccaatggaatagaatcgctgctttgtcatcacataaaccatcccctttaaatggttttgatcttttcaactatatggccctgatcgtttgtgcaaggagtaaagactacgcagaaattacctttaaaaagtttgctgatgaacttgaagaaaatcttaataagggtatcttcgcctttaaaggaaatgaacaaaagcgggtaacttgggaaggcatagctgtttggccgcacctgggacatacatttaaaggcttaaagaatcttggcaatataatgacaggttcagcctatccgggtctgtggaatgttagttatacaccaggtgatatgagttcaatggcggaagcttatactagaatttatatcaatacttgtcttgataataaagttaaggttcttagtgacgtaattagtggcggaaagtgtgacggtgttatttatcatcagaacagaagctgtaagctcatgagttttctgaatgtagaaactgctgatatcctccaaaaagaaaatggtttaccctatgtaagctttgatggagaccaaactgatcctcgtaacttttctcctgcccagtttgacacacgtatccaggccttagatgaaatgatgaagcagaataaggagggagtttccaatgagtag

SEQ ID NO: 172 - DNA, 1119 bp, Desulfosporosinus meridiei
  MISC_FEATURE (1)..(1119): strain ATCC BAA-275/DSM 13257/NCIMB13706/S10
atgagtagaattgaaactattattagtgaattatcttcaatttcaaataatccccgcaaggctatggaagattataaaaaagaaaccggtaaagggtcggtaggggttatgccttattatgcccctgaagaaataattcatgctgctggttttcttcccgtaggtatttggggaggacaaaagagtatttcaaaagcccgtgcctatttacctccctttgcttgttcaattatgcaatcagttatggaaatgcagcttgaaggggtatatgacgatttagaagcagtacttttccccgttccttgtgacactttaaaatgtctcagccaaaaatggaaaggaacatcacctgtcatcgtatttactcatcctcaaaacagaaaactcgaagcagccaataagtttcttgctgaggaatatcgacttgttcgtgaaaagctggaaacaatattgaatgtaaagattactgatgaagcactcaaccaaagtattgaaacttataacgaaaatcgtaaagtaatgcgtgaatttacggacctagctgctaattatcctcagattattgatcccagaatacgtcatgcaattataaaagctagattttttatggaaaaatctaaacataccgctatggtaaaagaattgaattcagagcttaaatcgttacctgttgaagcctttacaggtaaaaaggttgttttaacaggaattatggctgaacccaatgaagtattagacattttaaaagataacggttttgctgttgtggcagacgacctggcccaggaatccagactgttcagaaatgatgttccgtcaggtacagacccactatatcgattggctaaatggtggcaagaattcgatggttgttctcttgctacagatgcgaaaaaatcaagaggccccatgctgatggagatggttaaagggtctaaggccgatgcagttgtggtttgcatgatgaagttctgtgaccctgaagaatttgactatccaatctactatagacagtttgaagaagctggaattaagagcctatttatagaaattgacctggaaacaacatcctttgaacagactaaaaccagagttcaaagttttagtgaaatgctgtga

SEQ ID NO: 173 - DNA, 786 bp, Desulfosporosinus meridiei
  MISC_FEATURE (1)..(786): strain ATCC BAA-275/DSM 13257/NCIMB 13706/S10
atgtttacaatggggattgatattgggtcctcatcctcaaaggttgtaatacttgaagatggagttaatattatcgctggagaagtcattcagattggaacaggttcgacaggacctaaacgtgtactgaatgaagctctttccaaagcaggtcttaaattggaagacatggctaaaattattgctacaggctacggaagatcttctgtggaagaagcacacaaacaaattagcgaaatcagttgtcaggctaagggagttttctttttagttccttcagcaaaattaattattgatatcggcggtcaagatgttaaggcaattagacttgacagtaaaggcggcgttaagcagttttttatgaatgataaatgtgccgccggaacaggacgttttctcgatgttatgtcacgagtacttgaagttaatcttgatgaaatggcagaatacgatgctcgtgcaacagaacctgccacggtcagcagcacttgcacagtttttgcagaatctgaggtaatatctcagctttccaacggagttgctaaagagaatattattgcaggggttcaccagtcagttgctagcaaagcctgtggacttgcctatagatgtggggtggaagaggacattgttatgtgcggaggtgttgctaaggacttaggggttgtccgggcaataagcaaagaactaaaaaaacctgtcattgtagctcctaatccacaaattacagctgcccttggagctgctatctttgccttcgaagaagtcaggggagctaataaataa

SEQ ID NO: 174 - DNA, 1434 bp, Acidaminococcus fermentans
atgccaaagacagtaagccctggcgttcaggcattgagagatgtagttgaaaaggtttacagagaactgcgggaaccgaaagaaagaggagaaaaagtaggctggtcctcttccaagttcccctgcgaactggctgaatcttttcggctgcatgttgggtatccggaaaaccaggctgctggtatcgctgccaaccgtgacggcgaagtgatgtgccaggctgcagaagatatcggttatgacaacgatatctgcggctatgcccgtatttccctggcttatgctgccgggttccggggtgccaacaaaatggacaaagatggcaactatgtcatcaacccccacagcggcaaacagatgaaagatgccaatggcaaaaaggtattcgacgcagatggcaaacccgtaatcgatcccaagaccctgaaaccctttgccaccaccgacaacatctatgaaatcgctgctctgccggaaggggaagaaaagacccgccgccagaatgccctgcacaaatatcgtcagatgaccatgcccatgccggacttcgtgctgtgctgcaacaacatctgcaactgcatgaccaaatggtatgaagacattgcccgtcggcacaacattcctttgatcatgatcgacgttccttacaacgaattcgaccatgtcaacgaagccaacgtgaaatacatccggtcccagctggatacggccatccgtcaaatggaagaaatcaccggcaagaagttcgatgaagacaaattcgaacagtgctgccagaacgccaaccgtactgccaaagcatggctgaaggtttgcgactacctgcagtacaaaccggctccgttcaacgggttcgacctgttcaaccatatggctgacgtggttaccgcccgtggccgtgtggaagctgctgaagctttcgaactgctggccaaggaactggaacagcatgtgaaggaaggcaccaccaccgctcccttcaaagaacagcatcgtatcatgttcgaagggatcccctgctggccgaaactgccgaacctgttcaaaccgctgaaagccaacggcctgaacatcaccggcgttgtatatgctcctgctttcgggttcgtgtacaacaacctggacgaattggtcaaagcctactgcaaagccccgaactccgtcagcatcgaacagggtgttgcctggcgtgaaggcctgatccgcgacaacaaggttgacggcgtactggttcactacaaccggtcctgcaaaccctggagcggctacatgcctgaaatgcagcgtcgtttcaccaaagacatgggtatccccactgctggattcgacggtgaccaggctgacccgagaaacttcaacgcggctcagtatgagacccgtgttcagggcttggtcgaagccatggaagcaaatgatgaaaagaaggggaaataa

SEQ ID NO: 175 - DNA, 1140 bp, Acidaminococcus fermentans
atggctatcagtgcacttattgaagagttccaaaaagtatctgccagcccgaagaccatgctggccaaatataaagcccagggcaaaaaagccatcggctgcctgccgtactatgttccggaagaactggtctatgctgcaggcatggttcccatgggtgtatggggctgcaatggcaaacaggaagtccgttccaaggaatactgtgcttccttctactgcaccattgcccagcagtctctggaaatgctgctggacgggaccctggatgggttggacgggatcatcactccggtactgtgtgataccctgcgtcccatgagccagaacttcaaagtggccatgaaagacaagatgccggttattttcctggctcatccccaggtccgtcagaatgccgccggcaagcagttcacctatgatgcctacagcgaagtgaaaggccatctggaagaaatctgcggccatgaaatcaccaatgatgccatcctggatgccatcaaagtgtacaacaagagccgtgctgcccgccgcgaattctgcaaactggccaacgaacatcctgatctgatcccggcttccgtacgggccaccgtactgcgtgccgcttacttcatgctgaaggatgaatacaccgaaaagctggaagaactgaacaaggaactggcagctgctcctgccggcaagttcgacggccacaaagtggttgtttccggcatcatctacaacacgcccggcatcctgaaagccatggatgacaacaaactggccattgctgctgatgactgcgcttatgaaagccgcagctttgccgtggatgctccggaagatctggacaacggactgcatgctctggctgtacagttctccaaacagaagaacgatgttctgctgtacgatcctgaatttgccaagaatacccgttctgaacacgttggcaatctggtaaaagaaagcggcgcagaaggactgatcgtgttcatgatgcagttctgcgatccggaagaaatggaatatcctgatctgaagaaggctctggatgcccaccacattcctcatgtgaagattggtgtggaccagatgacccgggactttggtcaggcccagaccgctctggaagctttcgcagaaagcctgtaa

SEQ ID NO: 176 - DNA, 783 bp, Acidaminococcus fermentans
atgagtatctataccttgggaatcgatgttggatctactgcatccaagtgcattatcctgaaagatggaaaagaaatcgtggcgaaatccctggtagccgtggggaccggaacttccggtcccgcacggtctatttcggaagtcctggaaaatgcccacatgaaaaaagaagacatggcctttaccctggctaccggctacggacgcaattcgctggaaggcattgccgacaagcagatgagcgaactgagctgccatgccatgggcgccagctttatctggcccaacgtccataccgtcatcgatatcggcgggcaggatgtgaaggtcatccatgtggaaaacgggaccatgaccaatttccagatgaatgataaatgcgctgccgggactggccgtttcctggatgttatggccaatatcctggaagtgaaggtttccgacctggctgagctgggagccaaatccaccaaacgggtggctatcagctccacctgtactgtgtttgcagaaagtgaagtcatcagccagctgtccaaaggaaccgacaagatcgacatcattgccgggatccatcgttctgtagccagccgggtcattggtcttgccaatcgggtggggattgtgaaagacgtggtcatgaccggcggtgtagcccagaactatggcgtgagaggagccctggaagaaggccttggcgtggaaatcaagacgtctcccctggctcagtacaacggtgccctgggtgccgctctgtatgcgtataaaaaagcagccaaataa

SEQ ID NO: 177 - DNA, 1011 bp, Carboxydothermus hydrogenoformans
atgaaattaaactatttttgcagttactggccggtggaaatatccgaaggagcggggatttctacggtccgttatttcccgtccgatgaaagcaaagctccggtaaggcttcctgcttactgctgttcttatgccaggggaagccttgccgaaattgaagaagaaggagacggtgacttttggggatttgcccacagttgcgacacgatgcagagtttatacggcattactaagagtttactgggagacgaccgggtttttcttttcgttccgccggttgacttaaccaccgcttttgcccgggaatactaccgggaagctttaatttatctctggcgggaactttcccaaaaaagcggggttaatggtgaggaaaagttaaagcttacctgggaaaagttgaaggagttaagaaataaggttaaatctttggaaaacttgacgtcaattattccttcctccgaaatttttgagcttttaaaaaagcttcagaccctgccgctggatgaggctttggattacctcgaggccaaaaaagcggaatttaccagtttatctgtggctcaaaaggctatagggattattttaacgggagcggtagtcactaacagtaaactttaccttgctttagaacaacagggatttagagtagtttatgatgatacctgtaccggctttcgtcattttgctggagagatagaggataaagacgatattttggaggcaatagtttcttactacctttcaaagcccccctgtccctgcaggcataagggagtatgggcgagggcggagtatttaaaaaatctttatcataacaaaaatgcccgggccattgtacttttacaaaataaattttgtgacccctttgcctgggatgttccctatttagtggactactttaaaaaacagggagttccggttttagttttagaggtggaaggcggagaaatcggcgagcaaaataaaactcgcctccaggccttccgggaaagcgtgggtggagtgtaa

SEQ ID NO: 178 - DNA, 1215 bp, Carboxydothermus hydrogenoformans
atggctaaaaaaatctttaagcctcttaaggcttcagagaaaataaataaaattttaaaaaatcattatttaaaagcaaagtatttgccaacgcttggaaaattttttggttataaaaccgcctggattaccagcggagctccggtggaactactgcgggcctttggtatagagccggtttatccggagaattacggtgccatttgcggtgcccgcaaggtttcgccgagtctttgccaggtagcggaaaacaggggttattctctcgatttgtgttcttatgccaagagtaatctcggaagtatctggaatccgaaagaaagtccatttaacggcttaccccggccggatttactggtggtttgcaacaacatttgcgggacggttttaaagtggtacgaaactttaagccgggaatttaatattcccctttttatcattgataccccttttatcaccggtgaaccccaaccctggcaaatccagtatgtggccaaacagatagaaaaactggcgattgaactggaaaaatttttccggaaaaagttggatttaaaccgtttggaaaaagtaattctccttgccaatgagacggtggatttatggaaggggataagaaattttgccaaaaataaaccttcgccggtaaacgttaccgatttatttattaatctggggccaatggtggttttaaggggtaccgaagttgcccgggatttttacgaggaagtttaccgggaagtggaagaaaggtacaaagccggggttccggcggtagagggagaaaaataccgtttagtctgggacaacattcccatctggtacggactgtaccgtttttacggttattttgccgaaaggggagcggtttttgttaccgattcctataccggtggctgggcggtcaacataaaaaagggtcctcccttttatgcattagccgagacctatgccggcgtctttttaaatcgggatttagaatttcgcaaaaatcagttgcaatctttcattgaggaattttctgccgatggctttgtcatgcactccaatcgttcgtgcaaagcttattcttttgtgcaggaggaaatccggcgccaaatcatgaggtcactaggagtgccggggttaatagtggatgccgatatgaccgacagccggctttattccgaagaaacggttttaaaccgggtccaggctttcctggagagcctgtag

SEQ ID NO: 179 - DNA, 765 bp, Carboxydothermus hydrogenoformans
ttgtatcttggagttgatattggttcgcttacgaccaaggttgtcttaattgaccggggaaaaaatcttattgcttatcgttacagtaaaaccggacctgccggaaaggaaacggccgagcggttaattcaagaggttttgataaaagcgaatatttcccgggacgatattcagggaatagttgctaccggttacggcagggttctcttttccggaaaggagttttcggagataacctgtcaggcccgggggattgggcatttatacccggaggcaaaaacgattatcgatattggtggccaggatagcaaagtaatttctctgggaaaaaacggaaaggtactggactttgccatgaacgataaatgtgctgctggcaccggacgttttttggaggtgatgagtcaggcccttgaagttcgtctggaagagataggggaacttgccgaaaagagccaggaggcagctaagatatcttcggtttgtaccgtttttgccgaatcggaagtgatatccaatttatcccgggggcagagccgggaagcggtagcacggggaatttgtgaggcggtggcggcccgaacggctatactggcgcaaaaagtgggggtggtagaaccggtggtttttaccggaggggtggccaaaaatactggagttgtggcggctttggagcgaaagcttggggttaagttattaattccggaagattccacgattaccgcagctctgggggcggctttattagccgctgaaaattcttaa

SEQ ID NO: 180 - DNA, 786 bp, Oscillibacter valericigenes
atgaacaatatttacacgatgggcatcgacgtggggtccaccgcatccaagtgcctcatcctgaaagacggcagcgaaatcgttgccaagtctctggtagatgtgggcgcgggtaccagcggccctacccgtgctattgcggaggtactggaagccgcggggatgaagaaggaggacatggcttttattctggctaccggctatggccgcaattcactggacgacattgccgaccaccagatgagcgagctgagctgccatgccaaaggcgcgtttttcctgtttccggatgtccacaccgtcatcgacatcggcgggcaggatgtgaagattcttgagattgagaacggcgttatggtgaattttgccatgaatgacaagtgcgccgccgggacgggccggttcctggacgtgatggcccgggtgctggaggtgaaggtggaggatctggcggacctgggagcccagtccaccaagaatgtggagatcagctccacatgcaccgtgttcgctgagagcgaggtcatcagccagctggccaagggcagcgacaagcgcgacatcatccacggcatccacaagtctgtggcatcccgggtggttggccttgccaaccgtatcggtgtgcgggacgcggtggtgatgaccggcggcgtcgcccagaacggcggcgtggtctccgcgcttcaggaggcgttgggccatcccattcacacttcgcctctgacgcagtacaacggcgcgctgggcgcggcgttgtttgcatggcagaaggcaaccaaataa

SEQ ID NO: 181 - DNA, 1284 bp, Oscillibacter valericigenes
atggccgaaaacgaaaaagccactgcggccgctcccgaggcggctcctgttaagaaagctccgaagccggtcagccccggtacgcaggcgctgcgcgacgttgtcaccaaggtgtacgccgccgcgtgggatgcgaaaaaggcgggccgccccgtgggctggtcgtcttccaagttcccctgcgagatcgccgaggcgctgggccttgcagtcgtatatccggaaaaccaggctgccggtatcggcgcccagcacgatggccagcggatgtgtgaatctgccgagtccttgggcttcgacccagatatctgcggatacgcccggatttccctggcttattccgcgggcgttgagacgaccaatgagtcccgccgggttcccatgccggacttcgtgctgtgctgcaacaatatttgtaactgcatgaccaagtggtatgagaatattgcccggatgcacaacattcccctgattatgatcgacgtgccctataacaacgaggtcaccgtcagcgattcccaggtggcttacattcgcggccagttcgatgacgccattaagcagatggagaagattgccggcgtgaagttcgacgaaaagaagtttgaacaggcctgcgccaatgccaaccgcactgccaaggcgtggctgacggtctgtgactatttgcagtataagcccgctcccatgagcggcttcgatctgtttaaccatatggctgatgtggtgactgcccgcggcaaggtggagactgccgaggcgttcgagctgctggcaagcgagctggaacagcacgtaaaaaacggaaccagcaccgctccgttccccgagcagtaccgcgtcatgttcgagggcattccctgctggcccaacctaaggacgcttttcaagcccctgaaagccaacggcgtcaacgtcaccgccgtggtgtacgcgcccgcgttcggttttgtgtataacgggctggacgagatggcccgcgcatactgcaaggcccccaacagcgtgtgcattgagcagggcgtggactggcgcgagggcatctgtcgcgagaacaaggtagacggcgtgctggtgcactataaccgatcctgcaagccctggtccggctacatggccgagatgcagcgccgtttcaccaaggatctgggcgtcccctgcgccgggttcgacggagatcaggccgatccccgcaacttcaacgaggctcagtatgagacccgtgtccagggcctggtagaggctatggaggagaataaaaagcagaaggaggcccgggcatga

SEQ ID NO: 182 - DNA, 1143 bp, Oscillibacter valericigenes
atgagtatcgaaacgattgtaaaggagtttgccgacgttgcggccgacccgaaagcacagctgaagaaatacaaggcggagggcaaaaaatgcattggtgtgatgccgtattacgcgcccgaggagctggtggccgccgccggtatggtgccgtttggtatgtggggcagcaatgacaagaccatttctcgcgccaaggaatactgcgctacattttactgcaccatcgcccagctggatcttgagatgctgctggacggcaccatggatcttttagacggagtcatcacccccaccatctgcgacacgctccgtcccatgagccagaacatccgcgtggccatgggcgagaagctcccctgcattttcctggcccatccccagaaccgcaagcccgcttacggcaagaagttctgcctggaccaatatacccacatcaagactgagcttgagaagatcgccggcgcgcccatcaccgacgccgcactgtccgagaccatcaaggtctataataagagccgcgccgcccgccgtgagttcgtgaagctggtcagcgaccactgcgatgttatcacccccaccaaacgcagcgctgttttgaaagccgcgtggtttatgcccaaggcggagtacaccgagaagctgaaggccctcaacgcagagctgaaggctctgcctgtgtgcgactggaaggggaccaaggtggtcacctccggcatcatatgcgacaaccctaagcttctggagatcttcgaggagaacaaaatcgccatcgccgccgacgacgtggctcatgagtcccgctccttccgcgtagacgctcccgagaccggcgatcccatggaggcactcgcccagcagtttgccaatcaggattacgatgttctgctgtacgatgagcattccagcgagaaccgccggggcgagtttgtggccaagctggtgaaggacagcggcgccaaggggctggtcctgtttatgcagcagttctgcgacccggaggagatggagtatccctccctcaaaaaggcgctggacgaagccaagatcccccacatcaagctgggtgtggatcaacagatgcgggacttcggtcaggctcgcaccgcgattcaggcgtttgccgatgtgatctccctctaa

SEQ ID NO: 183 - DNA, 1269 bp, Desulfosporosinus orientis
  MISC_FEATURE (1)..(1269): strain ATCC 19365 / DSM 765 / NCIMB 8382 / VKM B-1628
atgactgatacagccaatatgagtgctaaagaattgttaggtttctatcaggaagaattgtatgaagaagcgagacaggccaaaaaagaaggaaaacttgtttgctggtcggcttccgttgctccttcggagttttgtgtagctatggacgtggccatgatctatcctgaaacccatgctgcagggatcggggccagaaaaggcgccttagatatgcttgaagttgccgatgaaaaagggtataacctggacacttgctcctatgccagagtgaatctgggttatatggaacttttaaaacaagaggctttaaccggaataaccccggagaaactggaaaaatctccggcggccagagtacccctgcctgattttgtcataacctgcaacaacatttgtaacaccttgcttaagtggtatgaaaatcttgccgttgagctaaatattccctgcatcgtcattgatgttccctttaatcacaccatgcccattccccagtatgctaaagactatattgcggaacagtttaaggaggcaattgctcagcttgaagagatttgcggcaagaaattcgactatgacaaattcttgcaagtccaggaacaaacccagcgctctgtggcccaatggaaccggattgcttctttgtcagggcataaaccatcccccttaaatggttttgatcttttcaactatatggccctgatcgtttgtgcccgcagcagggactgcgcagaaattacctttaaaaagtttgccgatgaactggaagacaatctaagcaaaggaatctacgcctttaaaggcaatgaacaaaagcgtatcacttgggaaggcatcgctgtttggccgcacctgggccatacctttaaaggcttaaagaatcttggcaatatcatgaccggttcagcctatcccggtttgtggaatctttcttatacgcccggtgatatgagttccatggcagaagcttacaccagaatttatatcaatacttgtctggataacaaagttaaggttcttagtgacatcatcagcggcggaaagtgtgacggtgttatttatcatcagaacagaagctgtaagctcatgagttttctcaatgtcgaaacggccgatatcctccaacaacaaaatcatttaccctatgtcagctttgatggagaccaaaccgatccccgtaactttgctcctgcccagtttgatacacggatccaagccttagatgaaatgatgaagcagaataaggagggagtttcccatgagtag

SEQ ID NO: 184 - DNA, 1119 bp, Desulfosporosinus orientis
  MISC_FEATURE (1)..(1119): strain ATCC 19365 / DSM 765 / NCIMB 8382 / VKM B-1628
atgagtagaattgaagcgattatcagtgaattatcttctattgccaataatccccgtaaggccatggaagattataagaaagaaacgggcaaagggtcggtagggattatgccttattatgctccggaagaaatcgttcatgccgccggttacctgcccgtaggaatttggggagggcaaaagagtatttctaaagcccgtgcttatttacctccttttgcttgttcaatcatgcaatccgttgtggaaatgcagctggaaggggtctataacgacttagcggcggtccttttccccgttccttgtgacactttaaaatgtctcagccaaaaatggaaaggcacatccccggtcatcgtcatgactcatcctcaaaaccgaaaactcgaagcagccaataagtttctggctgaggaatatcgccttgttcgtgaaaagctggaaaaaatcttaaatgttcagattaccgatgaggcactgaaccacagcattgatgtttataacgaaaatcgcaaggcaatgcgtgaatttacggacatagccgctaattatttgaacattattgatcccagaaagcgtcatgagattatcaaggccagattctttatggaaaaatccaaacataccgccttggtcaaagaattgaattccgagcttaaatctttacctgtggaagattttacaggcaaaaaggtgattttaaccggaatcatggctgaacccaatgaagtattagacattttgaaagagaatgattttgctgttgtggcagatgacctggcccaggaatccagactgttcaggattgatgttccggctggtccagacccactctaccgcttggctaaatggtggcaagaattcgacggttgttctctggctgtagatacgaaaaaattaagaggacccatgctgatgaatatggttaacgtggataaggccgatgccgtggtggtttgcatgatgaagttctgtgaccctgaagaatttgactatcccatctactacagacagtttgaagaagccggaattaagagcttatttatagaaattgacctggagccaacctcctttgaacagactaaaaccagagttcaaagttttcgtgaaatgctgtga

SEQ ID NO: 185 - DNA, 801 bp, Desulfosporosinus orientis
  MISC_FEATURE (1)..(801): strain ATCC 19365 / DSM 765 / NCIMB 8382 / VKM B-1628
atgtatactatggggattgatatcggttcctcatcctcaaaggttgtcatacttgaagatggagttaacctcatcgccggcgaagtcattcagattggaacaggctcgacaggtcctaaacgggtactggaggaagctcttgccaaaacaggtctcaccttggcagacatggctaaaattattgctaccggctacggccgatcttctgtggaagtatccgacaagcaaatcagcgaaatcagctgtcaggctaagggagtttactttttagttcctacagcaaaattaatcattgatatcggcggtcaggatgtgaaggccattagacttgaccgtataggcggcgtcaggcagttttttatgaatgataaatgtgccgccggaacaggacgttttctcgatgtgatgtcacgagtactggaagtggatctggatgaaatggcagaatacgatgcccgggccacagaacccgccacggtcagcagcacctgcacagtgtttgccgaatccgaggtaatatctcagcttgccaacggagttgctaaagagaatattattgccggggttcaccagtccgttgccagcaaagcctgtggactcgcctatcgatgcggggtggaagaggacgttgtgatgtgcggaggagttgctaaggacttaggagttgtccgggccatcagcaaagaactaaaaaaaccggtcattgtagctcctaatccccaaattacagccgcccttggcgctgccctatttgcttatgaagaagttatggaagctaataaattaaggaaagaggtatga

SEQ ID NO: 186 - DNA, 1236 bp, Peptostreptococcus anaerobius
  MISC_FEATURE (1)..(1236): CAG621
atgagtaacacaggtgcagttgaagaaaagccggcaaaagtattgttaggcgagatagttgcaaaacattataaggaagcttgggaagctaaagaaagaggcgaaaaagttggttggtgtgcttctaacttcccacaggaaatatttgaaacaatggatatcaaggttgtattccctgaaaaccaggcagcagcaatttctgctaagggtggtggacagaggatgtgcgaaatcgcagaaaacgaaggatattcaaacgacatatgtgcttacgctagaatatctctagcatacatggacgttaaagatgctccagagttaaatatgcctcagccagactttgttgcatgctgtaacaatatctgtaactgtatgatcaagtggtatgaaaatatagctaaagaactaaatatacctctaatccttgttgacgtgccatataacaatgactatgaagcaggcgatgacagagtagaatacttaagaggacagttcgatcacgctataaagcagttagaagacttaactggtaaaaagtgggatgaaaagaagttcgaagaagtaatggcaatatctcagagaacaggtagagcttggttaaaggctactggatatgctaagtacactccatcaccattctcaggatttgacgtattcaaccatatggcagttgctgtatgtgctagaggtaaggaagaatcagcaatagcatttgaaaagctagctgaagaatttgatgaaaatgtaaagactggtaagtctacattcaagggagaagaaaagtacagagtactatttgaaggtatagcttgttggccacacctaagacatacatttaagcagctaaaggattcaggagtaaacgtttgtggtactgtttatgcagatgcattcggatacatctacgacaatacttatgaattaatgcaggcttattgtggaactcctaatgcaatatcttatgaaagatcattagatatgagacttaaggttatagaagaaaataatatagacggtatgttgatacatataaacagaagctgtaagcagtggtctggtatcatgtacgaaatggaaagagaaataagagaaagaactggtataccaacagctacattcgatggtgatcaggctgacccaagaaacttctcagaagcacagtacgacacaagagtacagggtctaatagaagttatggaagcaaacaaagctgcaaagatgaaggaggaaaactag

SEQ ID NO: 187 - DNA, 1119 bp, Megasphaera elsdenii
  MISC_FEATURE (1)..(1119): DSM 20460
atgagtcagatcgacgaacttatcagcaaattacaggaagtatccaaccatccccagaagacggttttgaattataaaaaacagggtaaaggcctcgtaggcatgatgccctactacgctccggaagaaatcgtatatgctgcaggctacctcccggtaggcatgttcggttcccagaacccgcagatctccgcagctcgtacgtaccttcctccgttcgcttgctccttgatgcaggctgacatggaactccagctcaacggcacctatgactgcctcgacgctgttatcttctccgttccttgcgacactctccgctgcatgagccagaaatggcacggcaaagctccggtcatcgtcttcacacagccgcagaaccgtaagatccgcccggctgtcgatttcctcaaagctgaatacgaacatgtccgtacggaattggaacgtatcctcaacgtaaaaatctccgacctggctatccaggaagctatcaaagtatataacgaaaaccgtcaggttatgcgtgaattctgcgacgtagctgctcagtacccgcagatcttcactccggtaaaacgtcatgacgtcatcaaagcccgctggttcatggacaaagctgaacacaccgctttggtccgcgaactcatcgacgctgtcaagaaagaaccggtacagccgtggaatggcaaaaaagtcatcctctccggtatcatggcagaaccggatgaattcctcgatatcttcagcgaattcaacatcgctgtcgtcgctgacgacctcgctcaggaatcccgccagttccgtacagacgtaccgtccggcatcgatcccctcgaacagctcgctcagcagtggcaggacttcgatggctgcccgctcgctttgaacgaagacaaaccgcgtggccagatgctcatcgacatgactaagaaatacaatgctgacgccgtcgtcatctgcatgatgcgtttctgcgatcctgaagaattcgactatccgatttacaaaccggaatttgaagctgctggcgttcgttacacggtcctcgacctcgacatcgaatctccgtccctcgaacagctccgcacccgtatccaggctttctcggaaatcctctaa

SEQ ID NO: 188 - DNA, 1119 bp, Peptostreptococcus anaerobius
  MISC_FEATURE (1)..(1119): CAG621
atgagtaacttagaagaactatttggaaaacttgctgtatgtccattagagcagatagataaatatgttgctgatggtaagaaagttattggttgcgcgccagtatatgctccagaagaacttgtatacgcatcaggtatgattcctatggcaatatggggagcagagggtgaagtaactcttgcaaaagaatatttcccagctttctacgtatcaatcatcttaagacttttagatctaggtctagaaggcaagcttgataagatgtcaggaatgattctaccaggtctaagtgacggactaaagggacttagccagaactggaaaagagctgtaaagaatgttccagcattatatataggatatggacagaacagaaagatagaagctggtatagtttacaatgctagacagtatgaaaagctaaaagtacagttagaagaaatagctggaaagaagatagaagatgctcagatagaagaagcaatcgttttatacaacaagcacagaaaagctatgcaggcattctcagaccttgcagctaaacacttaaatacagttactcctagcctaagagctaaggtaatgtcaagtgcatgcctaatggacaaggctgaacatttagaaatagtagaagcaatcaacgctgaactttcagctatgccagaagaaaaatttgatggtaagaagattgtaactactggactactagctaacagtcctgaaatattaaagatatttgaagaatttaaacttggtattgttgctgacaacataaaccacgaatcaggacagtttgattatttagttgatgaagctactggtaacccaataaaggcgttgtctaagtggatttcagatattgaaggaagtactttgctatacgatccagaaaaactaagaggacagataatcatcgataaggctaaaaaatacgatgcagatggtgtagtatacctactatctaaattctctgattcagatgaatttgactacccaatcattagaaaacagctagaagaggctggatatatgcacatcttagttgaagtagatcagcaaatgactaacttcgaacaagcaaaaactgcattgcagacttttgcagacatgatatag

SEQ ID NO: 189 - DNA, 792 bp, Peptostreptococcus anaerobius
  MISC_FEATURE (1)..(792): CAG621
atgagtgatatatacacaatgggtattgacattggatcaacatcatctaaatgtgtagtgcttaagaatggtaaagatttagttagtagcggcgtcgtcaatcttggcgccggtactaaaggtgccgatcaggttatagaaaaggtactagctgactgtggtatcaagttcgaagatctgaatgtgattgtttccacaggatatggtagaaattcttacgacagtgcaaagaagactatgagtgaacttagctgtcatgctaagggtggtacatatatcttcggacctgtaagaactattatagatataggcggacaggacataaaggtactaaaactaaatgacaaaggtatgatgacaaatttcttgatgaatgataaatgtgcagctggtacaggtagattcttagaggttatggctggagtacttgatgttaagctagcagaactaggtgacttagacaagttagcaactgaaaaaacaccaatatcttcaacttgtacagtatttgcagaatcagaagtaatatcttgtatggctaagaaaatacctattcctaatataattaggggtatacacgcttctgttgctacaagagttgcaggtcttgctaagagaggtggattaacaactccagtcgctatgactggtggtgttactaagaactcaggaatagtaagggcacttagcgaagagttagaaacagatatcatgatttcggaaatttctcagttggcaggcgcaattggagcggcattgtacgcttacgatgagtatctgaaggaaaattag

SEQ ID NO: 190 - DNA, 777 bp, Chloroflexus aggregans
  MISC_FEATURE (1)..(777): strain MD-66 / DSM 9485
atgagcgatgaaacgcttgtgctcagcactatcgaaggccccgttgcaatccttacgctcaatcgaccacaagcactcaatgcccttagccctgccctcatcgacgcactcatccgccatcttgagcattgcgataacgacgatacgatccgggtgatcattatcaccggcgccggtcgcgcctttgccgccggcgccgacatcaaggcgatggccgatgcgacgccgatcgatatgcttacaaccgatatgattgcccgctgggcgcggattgcggcggtgcgcaaacccgtgatcgcagccgtgaacggatttgccctcggtggtggctgcgagttggctatgatgtgtgacatcattcttgccagtgaaacagcccaattcggtcaacccgaaatcaacatcggcattatccccggcgccggtggcacccaacgcctgacccgcgcaattggcccataccgtgcaatggagatggtcttaaccggtgctaccatcagtgcccaagaagcttacgcctacggcctggtgaatcgggtatgcccacccgatagcctgcttgatgaagcccgccggttggcccagaccattgcagccaagccgccgctcgctgtgcgtttagccaaggaagccgtgcgcgctgcggctgaaacgaccgtgcgtgaagggttagccattgaattgcgtaacttttatctgctctttgccagtgccgatcagaaagagggcatgcgagcctttatcgaaaagcgtacagccaacttcagtggtcgctaa

SEQ ID NO: 191 - DNA, 774 bp, Marivirga tractuosa
  MISC_FEATURE (1)..(774): strain ATCC 23168/DSM 4126/NBRC 15989/NCIMB 1408/VKM B-1430/H-43
atggaattcataaaagtaaacacacaatataaaaagcatattgcgctcatcaatcttaacagacctaaagaattaaatgccttgaacttacagttaatgactgaattgaaggacactttaaaggtcttggatgaggatgaaaatgttagagttataattttaacaggtaatgagaaggcttttgccgctggagcagacattaagcaaatggcaggtaaaacggctattgacatgctcaatgttgatcaattcagcacttgggatcaaatcaaaaaaacaaagaagccattgattgcagccgtttcaggatttgcattgggcggtggttgcgaattagcgatgacttgcgatatgattgtagcgtcagaatctgctaaattcggtcagcctgaaataaaaatcggagtaatgccgggagcaggtggtacacaaaggttaactagggcaattggtaaagccaaagcgatggaattagtcttgactggtaattttattagtgcagaggaagcaatgcattatggcttagttaataaagttgttcctacagagatgtatctggaagcagctgctgaactggctgagcaaatagcacaaatgtctcctgtagcagctaagttggcaaaagaatcagttaacagggcttttgaaacgcatttggacgaaggcttgcactttgagagaaaaaacttctatttaacatttgcttcagaagatcagactgaaggtatggaagcttttgtagagaaaagaaagcctgaattcaaggggaaataa

SEQ ID NO: 192 - DNA, 774 bp, Marinithermus hydrothermalis
  MISC_FEATURE (1)..(774): strain DSM 14884 / JCM 11576 / T1
atgtacgagaacctcatcgtggagacgctcgagggcggcgtggggctcattcgcatccaccggcccaagcgcctcaacgccctgaaccaggccaccatggacgagatcgtccgcgcagtacgcgcgtttgaagcggatgacgcggtgcgcgcgatcgtcctcacgggggacgagcgggcgttcgccgcgggcgcggacgtcaccgagatggacggcgcgaacgtgccggagatgctctccgggtaccgcttcgagcagtgggagaccctccggcgcaccacgaaacccttgatcgccgcggtctcggggttcgcgctcgggggcgggctcgagctcgcgatgctgtgcgacatcatcgtagcctcggagaccgcgcggctcggccagcccgagatcaacctcgggatcatgccgggggcgggcggcacgcaacggctcacgcggcaggtgggcaagtacctcgcgatggagatggtcctcacggggcgcatgctcaccgcggaggaggcgtaccgtcacggcctggtgaaccgggtcgtcccggtcgagttctacctggaggaagccatccagatcgcgcgggagatcgcgaagaaagccccggtggcggtgcgcctggccaaggacgcgatcctcaaggcagaggacacgccgctcgaggtgggcctcgcgtacgagcgccacaacttctacctgctcttcggcaccgaggacaagcaagaagggatccgcgctttcctcgagaagcgcaagcccgaatggaaagggaggtag

SEQ ID NO: 193 - DNA, 780 bp, Chitinophaga pinensis
  MISC_FEATURE (1)..(780): strain ATCC 43595 / DSM 2588 / NCIB 11800 / UQM 2034
atgcaaccacaatttataatcatacaccggcaggtagccccatatgtggctcatatacagttaaaccgccccaaagaactcaatgcactgaaccttgaactgatgattgagctcagggatgcattaaaaatgttggatgcggatgacaatgttcgtgcaatcgtcatcagcggtaatgaaaaagcattcgctgcaggcgcggatatcaaacagatggcggggaaaactgccatggacatgtataacattgaccagttcagcacctgggacacaataaaaaaaactaaaaagccgttgattgcggcagtaagcggcttcgcgctgggagggggatgtgagctggtgatgctatgcgatatgatagtagccagtgaaacagcgcggttcggacagccggaaataaaaattggcgtcatgcctggcgcaggtggtacacaacgcctgacccgcgccgtaggtaaagccctggccatggaaatggtattgacaggtcgctttatcactgcacaagaagctgcacgtgcaggtcttatcaaccgggtaataccggtggaacttttcctgcaggaagccatccggctggcgactgaagtagctgcgcttagtccgttggcagtaaagatggctaaagaatctgtactgaaagcatttgatagctccctcgaagaaggactacattttgaacgtaaaaacttttatctgctgtttgcctctgaagatcagaaagaaggcatgcaggcttttgttgataagagatcacctgtttttaaaggaaaataa

SEQ ID NO: 194 - DNA, 777 bp, Megasphaera elsdenii
  MISC_FEATURE (1)..(777): DSM 20460
gtgtatactctcggaatcgacgttggttcttcttcttccaaggcagtcatcctggaagatggcaagaagatcgtcgcccatgccgtcgttgaaatcggcaccggttcgaccggtccggaacgcgtcctggacgaagtcttcaaagataccaacttaaaaattgaagacatggcgaacatcatcgccacaggctatggccgtttcaatgtcgactgcgccaaaggcgaagtcagcgaaatcacgtgccatgccaaaggggccctctttgaatgccccggtacgacgaccatcctcgatatcggcggtcaggacgtcaagtccatcaaattgaatggccagggcctggtcatgcagtttgccatgaacgacaaatgcgccgctggtacgggccgtttcctcgacgtcatgtcgaaggtactggaaatccccatgtctgaaatgggggactggtacttcaaatcgaagcatcccgctgccgtcagcagtacctgcacggtttttgctgaatcggaagtcatttcccttctttccaagaatgtcccgaaagaagatatcgtagccggtgtccatcagtccatcgccgccaaagcctgcgctctcgtgcgccgcgtcggtgtcggtgaagacctgaccatgaccggcggtggctcccgcgatcccggcgtcgtcgatgccgtatcgaaagaattaggtattcctgtcagagtcgctctgcatccccaagcggtgggtgctctcggagctgctttgattgcttatgataaaatcaagaaataa

SEQ ID NO: 195 - DNA, 1287 bp, Megasphaera elsdenii
  MISC_FEATURE (1)..(1287): DSM 20460
atgagtgaagaaaaaacagtagatattgaaagcatgagctccaaggaagcccttggttacttcttgccgaaagtcgatgaagacgcacgtaaagcgaaaaaagaaggccgcctcgtttgctggtccgcttctgtcgctcctccggaattctgcacggctatggacatcgccatcgtctatccggaaactcacgcagctggtatcggtgcccgtcacggtgctccggccatgctcgaagttgctgaaaacaaaggttacaaccaggacatctgttcctactgccgcgtcaacatgggctacatggaactcctcaaacagcaggctctgacaggcgaaacgccggaagtcctcaaaaactccccggcttctccgattccccttccggatgttgtcctcacttgcaacaacatctgcaataccttgctcaaatggtatgaaaacttggctaaagaattgaacgtacctctcatcaacatcgacgtaccgttcaaccatgaattccctgttacgaaacacgctaaacagtacatcgtcggcgaattcaaacatgctatcaaacagctcgaagacctttgcggccgtcccttcgactatgacaaattcttcgaagtacagaaacagacacagcgctccatcgctgcctggaacaaaatcgctacgtacttccagtacaaaccgtcgccgctcaacggcttcgacctcttcaactacatgggcctcgccgttgctgcccgctccttgaactactcggaaatcacgttcaacaaattcctcaaagaattggacgaaaaagtagctaataagaaatgggctttcggtgaaaacgaaaaatcccgtgttacttgggaaggtatcgctgtctggatcgctctcggccacaccttcaaagaactcaaaggtcagggcgctctcatgactggttccgcttatcctggcatgtgggacgtttcctacgaaccgggcgacctcgaatccatggcagaagcttattcccgtacatacatcaactgctgcctcgaacagcgcggtgctgttcttgaaaaagttgtccgcgatggcaaatgcgacggcttgatcatgcaccagaaccgttcctgcaagaacatgagcctcctcaacaacgaaggcggccagcgcatccagaagaacctcggcgtaccgtacgtcatcttcgacggcgaccagaccgatgctcgtaacttctcggaagcacagttcgatacccgcgtagaagctttggcagaaatgatggcagacaaaaaagccaatgaaggaggaaaccactaa

SEQ ID NO: 196 - DNA, 1119 bp, Megasphaera elsdenii; identical to SEQ ID NO: 187
  MISC_FEATURE (1)..(1119): DSM 20460

SEQ ID NO: 197 - DNA, 777 bp, Chloroflexus aurantiacus
  MISC_FEATURE (1)..(777): strain ATCC 29364 / DSM 637 / Y-400-fl
atgagtgaagagtctctggttctcagcacaattgaaggccccatcgccatcctcaccctcaatcgcccccaggccctcaatgcgctcagtccggccttgattgatgacctcattcgccatttagaagcctgcgatgccgatgacacaatccgcgtgatcattatcaccggcgccggacgggcatttgctgccggcgctgacatcaaagcgatggccaatgccacgcctattgatatgctcaccagtggcatgattgcgcgctgggcacgcatcgccgcggtgcgcaaaccggtgattgctgccgtgaatgggtatgcgctcggtggtggttgtgaattggcaatgatgtgcgacatcatcatcgccagtgaaaacgcgcagttcggacaaccggaaatcaatctgggcatcattcccggtgctggtggcacccaacggctgacccgcgcccttggcccgtatcgcgcaatggaattgatcctgaccggcgcgaccatcagtgctcaggaagctctcgcccacggcctggtgtgccgggtctgcccgcctgaaagcctgctcgatgaagcccgtcggatcgcgcaaaccattgccaccaaatcaccactggctgtacagttggcgaaagaggcagtccgtatggccgccgaaaccactgtgcgcgaggggttggctatcgagctgcgtaacttctatctgctgtttgccagtgctgaccaaaaagaggggatgcaggcatttatcgagaaacgcgctcccaacttcagtggtcgttga

SEQ ID NO: 198 - DNA, 777 bp, Ruegeria pomeroyi
  MISC_FEATURE (1)..(777): DSS-3
atggcctttgagacgatcatcgtcgaagttgaagaccacgtagccctgatcaggctgaaccgtcccgatgcgctcaatgcgctcaacacccagttgctgggcgagttgtgtaccgcgctggaagaggccgacggcaatgacaaggtgcgctgcatcgtcatcaccggcagcgacaaggcatttgccgccggggccgatatccgcgagatgtcccaaaagacctatgtcgaggtgtatagcgagaacctgttcgcggccgccaacgaccgtgtcagcgccatccgcaagccgatcatcgccgcagtggcgggctatgcgctgggcggtggctgtgaactggcgatgctgtgcgatttcatcatcgcggcggacaccgcaaagttcggccagcccgagatcaacctgggcgtgatcgccggtatcggcggcacccagcgtctgacccggctggtgggcaagtccaagtcgatggacctgaacctgaccgggcggttcatggatgccgaagaggccgagcgcgccgggctggtcagccgcgtggttccggccaagaagctggtcgaagaggcgctgagcgcagcccagaagatcgccgagaaatcgatgatctcggcctatgcggtcaaggaggcggtcaaccgctcttacgagaccacgctgagcgaggggctgctgttcgagcgccgggtgttccattcgatgttcgccaccgaagatcagaaggaaggcatggccgctttcctcgagaagcgggcggcacagttccgcgacaagtga

SEQ ID NO: 199 - protein, 132 aa, Escherichia coli
MSTTHNVPQGDLVLRTLAMPADTNANGDIFGGWLMSQMDIGGAILAKEIAHGRVVTVRVEGMTFLRPVAVGDVVCCYARCVQKGTTSVSINIEVWVKKVASEPIGQRYKATEALFKYVAVDPEGKPRALPVE

SEQ ID NO: 200 - protein, 132 aa, Klebsiella oxytoca
  MISC_FEATURE (1)..(132): 10-5245
MTTTDLAPKGELVLRTLAMPADTNANGDIFGGWLMSQMDIGGAIMAKEIAHGRVVTVRVDGMTFLRPVAVGDVVCCYANCVKRGNTSITINMEVWVKKVSSEPIGQRYKATEALFIYVAVDNQGKPRALPTL

SEQ ID NO: 201 - protein, 133 aa, Cronobacter turicensis
MTTEQTTPQGELVLRTLAMPADTNANGDIFGGWLMAQMDIGGAILAKEIAHGRVVTVRVDGMTFLRPVAVGDVVCCYARCVKRGNTSVTINIEVWVKKVSSEPLGQRYRATEALFIYVAVDDNGKPRPLPPVA

SEQ ID NO: 202 - protein, 133 aa, Citrobacter freundii
MTTTNNTPQGELVLRTLAMPADTNANGDIFGGWLMSQMDIGGAIQAKEIAHGRVVTVRVEGMSFLRPVAVGDVVCCYARCVKRGTTSISINIEVWVKKVASEPIGQRYKATEALFIYVAVDKDGKPRPIPTLA

SEQ ID NO: 203 - protein, 130 aa, Salmonella enterica
MDNTPQGELVLRTLAMPADTNANGDIFGGWLMSQMDIGGAILAKEIAHGRVVTVRVEGMTFLRPVAVGDVVCCYARCVKRGTTSISINIEVWVKKVASEPIGQRYKATEALFIYVAVDPDGKPRPLPVQG

SEQ ID NO: 204 - protein, 133 aa, Shigella flexneri
  MISC_FEATURE (1)..(133): 1235-66
MTTTNNTPQGELVLRTLAMPADTNANGDIFGGWLMSQMDIGGAIQAKEIAHGRVVTVRVEGMSFLRPVAVGDVVCCYARCVKRGTTSISINIEVWVKKVASEPIGQRYKATEALFIYVAVDKDGKPRPIPKQV

SEQ ID NO: 205 - DNA, 399 bp, Escherichia coli
atgtctacaacacataacgtccctcagggcgatcttgttttacgtactttagccatgcccgccgataccaatgccaatggtgacatctttggtggttggttaatgtcacaaatggatattggcggcgctattctggcaaaagaaattgcccacggtcgcgtagtgactgtgcgggttgaaggaatgactttcttacggccggttgcggtcggcgatgtggtgtgctgctatgcacgctgtgtccagaaagggacgacatcggtcagcattaatattgaagtgtgggtgaaaaaagtagcgtctgaaccaattgggcaacgctataaagcgacagaagcattatttaagtatgtcgcggttgatcctgaaggaaaacctcgcgccttacctgttgagtaa

SEQ ID NO: 206 - DNA, 399 bp, Klebsiella oxytoca
  MISC_FEATURE (1)..(399): 10-5245
atgacaacaacagatcttgcgccgaagggcgaattggttttacgcaccctggcgatgccggcggacaccaacgcaaacggcgatattttcggcggctggctgatgtcgcaaatggatattggcggggccattatggccaaagaaattgcccacggtcgcgtcgtgaccgtgcgcgtcgacggcatgacctttttgcgcccggtggcggtcggcgacgtcgtgtgctgctacgccaactgcgtgaagcgcggcaatacgtcgataactatcaatatggaagtgtgggtcaagaaagtgtcgtctgagcccatcggccagcgctacaaagccaccgaagcgctgtttatctacgtcgcggtggataatcagggaaaaccgcgcgcactgccgactctgtga

SEQ ID NO: 207 - DNA, 402 bp, Cronobacter turicensis
atgacgacagagcaaaccacgcctcaaggtgaactggttttacgtaccctggcgatgcccgccgataccaacgccaatggcgatatttttggcggctggctgatggcccagatggacattggcggcgcgatccttgccaaagagatagcccatggccgcgtggtgacggtacgcgttgacggcatgacgttcctgcgcccggtcgcggttggcgatgtggtgtgctgttatgcccgttgcgtgaagcgcggcaatacatcggtgacgattaatattgaagtgtgggtgaagaaggtttcttccgagccgcttggccagcgctaccgcgcgaccgaggcgctgttcatttatgttgcggtcgatgacaacggcaaaccgcgcccgctgccgcctgtggcgtga

SEQ ID NO: 208 - DNA, 402 bp, Citrobacter freundii
atgacaacaacgaataacactccccagggtgaactggttttacgcactctggccatgcctgccgataccaacgcgaacggtgatatttttggcggctggctgatgtcacaaatggatataggtggcgcgattcaggccaaagagatcgcacatggtcgtgtggtaactgtgcgggttgaaggaatgagctttttgcgcccggtcgccgtaggtgatgtagtgtgttgctatgctcgctgtgtgaaacgcgggacaacctcaatcagcatcaatattgaagtttgggtgaagaaagtcgcttctgaacctattggccagcgttataaggccaccgaagctctgtttatctacgttgccgttgataaagacgggaaaccgcgtccaatccccacgttggcctga

SEQ ID NO: 209 - DNA, 393 bp, Salmonella enterica
atggataatactcctcagggcgagctggttttacgtacattggccatgcctgccgataccaatgcgaacggcgatatttttggcggctggctgatgtcgcaaatggatattggcggcgcgatactggccaaagagatcgcgcacggtcgggttgtaaccgtacgcgtggaaggaatgacatttctgcgccccgtcgcggttggcgatgtcgtatgctgctacgcgcgctgcgttaaacgcggtacgacgtctattagcataaatattgaagtctgggtgaaaaaagtcgcgtcagaaccgattgggcagcgctacaaggccaccgaggcgctgtttatttatgttgccgtcgatccggacggtaaacctcgcccgctcccggttcagggttaa

SEQ ID NO: 210 - DNA, 402 bp, Shigella flexneri
  MISC_FEATURE (1)..(402): 1235-66
atgacaacaacgaataacaccccccagggtgaactggttttacgcactctggccatgcctgccgataccaatgctaacggtgatatttttggcggctggctgatgtcacagatggatattggtggcgctattcaggccaaagagatcgcacacggtcgcgtggtgacggtgcgagttgaaggaatgagctttttgcgcccggttgccgtgggtgatgtggtctgttgctacgcacgctgcgtaaaacgcgggacgacgtcaatcagcattaatattgaagtctgggtgaagaaagtcgcttcggaacctattggccagcgttacaaagccactgaagccctgtttatctacgtcgctgtagataaagacggtaaaccccgtccgatacctaaacaggtctga

SEQ ID NO: 211 - protein, 554 aa, Ilyobacter polytropus
  MISC_FEATURE (1)..(554): DhaB1 protein
MKSKRFEVLKERPVNKDGFISEWIEEGLIAMESPNDPNPSLKIENGQITELDGKSREEFDMIDRFIADYAINMENAEKAMKMSSMEISKKLVDINVSRDEVLEITTGITPAKIIKVMEHMNVVEMMMAVQKMRARKTPSNQCHVTNLRDNPVLIAADAAEASVRGFDEQETTIGIVRYAPFNAISIFVGSQVGRGGILTQCSVEEATELELGMKGFTSYAETVSVYGTEQVFIDGDDTPWSKAFLASAYASRGLKMRFTSGTGSEALMGNAEGKSMLYLEARCIYVTRGSGVQGLQNGSVSCIGMPGSLPGGIRAVLAENLIAMLLDLECASANDQTFSHSEYRRTARTLMQMLPGTDFIFSGYSAVPNCDNMFAGSNFDAEDFDDYNALQRDLKIDGGLKPVTEDEIVKVRNKAARAIQGLFKELDLPEITDEEVEAATYAHGSVDMPARNVVEDLKAAEELLSSGITGVDLVKGLSRSGFDDVAEHVLGMLKQRVSGDYLQTSAILDKGFKIKSAINDRNDYMGPGSGYRISEERWEEIKNIPSAIKPESIE

SEQ ID NO: 212 - protein, 187 aa, Ilyobacter polytropus
  MISC_FEATURE (1)..(187): DhaB2 protein
MENKFVPSVKIEEIGEAKKGSRSEEVVIGLAPAFKKFQHKTITDVPHDEVLTELIAGIEEEGLKARIVRVTRTSDVSFMALDAAKLSGSGIGIGIQSKGTTVIHQKDLLPLNNLELFPQAPLLTPETFRLIGKNAAKYAKGESPNPVPVASDQMARPKYQAKAALLHIKETKHVVQHGKPVEIKYEF

SEQ ID NO: 213 - protein, 143 aa, Ilyobacter polytropus
  MISC_FEATURE (1)..(143): DhaB3 protein
MNIDVKNINPISDYPLGEKRKEWLKTSTGKTLDEITLENVINGDIKPEDIRISPETLKLQGEIAKKGNRPTITKNFERASEMVAIPDDKILATYNALRPYRSSKEELFEIADELESKYSAVVISAFIKEAAEVYEQRGQLRKD

SEQ ID NO: 214 - DNA, 1665 bp, Ilyobacter polytropus
  Gene (1)..(1665): dhaB1 gene
atgaaatcaaaaagatttgaagtattgaaggaacgtcctgtaaataaagatggctttataagtgaatggatagaagaaggactaatcgcaatggaaagtcctaacgatcctaatccaagtttgaaaatagaaaatggtcaaataacagagttagacggtaaaagcagagaagaatttgacatgatcgacagatttatagcagattatgcaataaatatggaaaatgctgaaaaagctatgaaaatgtcatctatggaaatatctaaaaaactagtagacataaatgtatcaagagatgaagtgctggaaataacaacaggaattaccccagcaaaaataattaaagttatggaacacatgaatgttgtagagatgatgatggccgtacaaaaaatgagagccagaaaaactccttccaatcagtgtcatgtaactaacttgagagacaatcctgtattaattgccgctgatgctgccgaagcgtcagtaagaggttttgatgaacaggagactacaatcggtatagtaagatatgcacctttcaatgccatctcaatatttgtaggttcacaagtaggtagaggaggaatactgactcagtgttctgtagaagaagctactgaattagagcttggaatgaaaggattcacaagttatgcagaaacagtgtctgtatatggtacagagcaagtgtttatagacggtgacgacactccttggtcaaaagccttccttgcttcagcatatgcatcaagaggattaaaaatgagatttacatctggaactggttcagaggctcttatgggaaatgctgaagggaaatcaatgctttaccttgaagcaagatgtatctacgtaacaagagggtctggagtacaaggactacaaaatggttctgtaagctgcatagggatgcctgggtcactacctggaggaataagggctgtactggctgaaaacctgatagcaatgttacttgacttagaatgtgcatcagcaaatgaccagacattctctcactcagaatatagaaggacagcaagaactctaatgcagatgcttcctggaacagacttcatattctcaggatatagtgccgtaccaaactgtgataacatgtttgctggatcaaattttgatgcagaggattttgatgactataatgctcttcagagagaccttaaaatagacggtggtttaaaacctgtaactgaagatgagattgtcaaagtaagaaataaagcagccagagcaatacaggggttattcaaagaacttgatcttcctgaaataacagatgaagaagtggaagcagcaacatatgcccacggaagtgttgatatgcctgcaagaaatgtggttgaagatttaaaagcggcagaagaacttttaagctctggaataacaggagtagatcttgttaaaggacttagcagaagcggatttgacgatgtagctgagcatgttttaggtatgttaaaacagagagtttcaggagattacctgcaaacttcagctatattagacaaaggctttaaaataaagagtgccataaacgatagaaatgattacatgggtcctggaagcggatatagaataagcgaggaaagatgggaagagatcaaaaatatcccatcagctataaaaccagaaagtatagaatag

SEQ ID NO: 215 - DNA, 564 bp, Ilyobacter polytropus
  Gene (1)..(564): dhaB2 gene
atggaaaataaatttgtaccatctgtaaagatagaagaaatcggagaagcaaaaaaaggaagcagatctgaagaagtagttataggactggctcctgcatttaaaaaatttcaacataaaacaataacagatgtccctcacgatgaagtcctgactgaacttatcgcaggtatagaggaagagggattaaaggcaagaatcgtaagagtaacaagaacttctgatgtttcatttatggcgctggatgctgcaaagttaagtggttctggaataggaataggaattcagtcaaagggaacaacagtaatccaccaaaaggatctgcttcctctaaacaatctagaacttttcccacaggctccactattaacacctgaaacattcagattaataggaaaaaatgctgcaaaatatgcaaagggagaatctccaaatccagtacctgtagccagtgaccagatggcgagacctaaatatcaggcaaaagcagcattactacatataaaagagacaaaacatgtcgttcaacacggaaaaccagtagagataaagtatgaattttag

SEQ ID NO: 216 - DNA, 432 bp, Ilyobacter polytropus
  Gene (1)..(432): dhaB3 gene
atgaatatagatgttaaaaatataaatccaatctctgattatccattaggagaaaagagaaaagaatggttgaaaacatccacaggtaaaactttggatgaaataactttagaaaatgtaataaatggagatataaagcctgaagatataagaatctcacctgaaactctaaaattacagggagagatagcaaagaaaggtaacaggccaactataacaaagaactttgaaagagccagtgaaatggttgccattccagatgataaaatattagcaacttacaacgctttgagaccttacagatcttcaaaggaagaattatttgaaatagccgatgaactagaaagtaagtattcagctgttgtaatatctgcatttatcaaggaagccgcagaagtttatgaacaaagaggtcaacttagaaaagattag

SEQ ID NO: 217 - protein, 607 aa, Klebsiella pneumoniae
  MISC_FEATURE (1)..(607): gdrA protein
MPLIAGIDIGNATTEVALASDDPQARAFVASGIVATTGMKGTRDNIAGTLAALEQALAKTPWSVSDVSRIYLNEAAPVIGDVAMETITETIITESTMIGHNPQTPGGVGVGVGTTIALGRLATLPAAQYAEGWIVLIDDAVDFLDAVWWLNEALDRGINVVAAILKKDDGVLVNNRLRKTLPVVDEVTLLEQVPEGVMAAVEVAAPGQVVRILSNPYGIATFFGLSPEETQAIVPIARALIGNRSAVVLKTPQGDVQSRVIPAGNLYISGEKRRGEADVAEGAEAIMQAMSACAPVRDIRGEPGTHAGGMLERVRKVMASLTDHEMSAIYIQDLLAVDTFIPRKVQGGMAGECAMENAVGMAAMVKADRLQMQVIARELSARLQTEVVVGGVEANMAIAGALTTPGCAAPLAILDLGAGSTDAAIVNAEGQITAVHLAGAGNMVSLLIKTELGLEDLSLAEAIKKYPLAKVESLFSIRHENGAVEFFREALSPAVFAKVVYIKEGELVPIDNASPLEKIRLVRRQAKEKVFVTNCLRALRQVSPGGSIRDIAFVVLVGGSSLDFEIPQLITEALSHYGVVAGQGNIRGTEGPRNAVATGLLLAGQAN

SEQ ID NO: 218 - protein, 117 aa, Klebsiella pneumoniae
  MISC_FEATURE (1)..(117): gdrB protein
MSLSPPGVRLFYDPRGHHAGAINELCWGLEEQGVPCQTITYDGGGDAAALGALAARSSPLRVGIGLSASGEIALTHAQLPADAPLATGHVTDSDDHLRTLGANAGQLVKVLPLSERN

SEQ ID NO: 219 - protein, 607 aa, Ilyobacter polytropus
  MISC_FEATURE (1)..(607): gdrA protein
MKIIVGVDIGNATTEVALAKVDNIECKFLSSALHETTGLKGTKDNVLGIKRAIKKAMKRADLKNADLSLIRINEATPVIGDVSMETITETIITESTMIGHNPSTPGGIGLGIGETILFQELGNFENDKDYIVIVEKSFSFLEVAHRINEAFKNGCKIKGAIIQKDDGVLINNRLINKIPIVDEVLFVKKVPTGMKAAVEVAPQGKIIEVISNPYGIATIFSLTSEETKKIVPISKALIGNRSGVVIKTPHGDVKEKVIPAGRIQIDGNYRSKSVNIEEGSKRIMKALGSIEHVQDINGESGTNIGGMLKNVKSVMGNFTNESIDNIKIKDILAVDTFVPQKIKGGIAEEFVFENAVGIAAMVNTKKNQMSEVAKEIEKELGVKVEVGGVEADMAITGALTTPGTGTPLVIVDIGAGSTDACSIDRYGNKELVHLAGAGNMTTLLIQKELGIEDFNLAEDIKKYPLAKVESLFYIRHEDGNVQFFENSLSPKVFAKNVLIKEGELIPIDLDMSLEKIRIIRRSAKRKIFITNVLRSLRKVSHTKNIRDFEFVVIVGGSALDFEISQMITEALSEYGIVAGCGNIRGTEGPRNAVATGLVMGVNDGQQA

SEQ ID NO: 220 - protein, 107 aa, Ilyobacter polytropus
  MISC_FEATURE (1)..(107): gdrB
MDNRPNITLFCSDNIDREYINEILWGIEEEEIPYLLKIVPSKEVVKENYVSGTLEIGIGVLENGDALLTTRKYDKEYIQKANIFVEKNKLRDLGSNGARLVKGLPLR

SEQ ID NO: 221 - DNA, 1824 bp, Ilyobacter polytropus
  Gene (1)..(1824): gdrA gene
atgaagatcatagtgggtgtagatattggaaatgctacaacagaagtagctttggcaaaggtagacaatatagaatgtaagtttttatccagtgccttacatgaaacaacaggtttaaaaggtactaaagataatgttttgggaataaaaagagccattaagaaggcaatgaaaagagctgatttaaaaaatgcagatttatctttaatcaggataaatgaagctactcctgttataggagacgtttctatggaaactataacagaaacaataattacagagtctactatgattggacataacccttcaactcctgggggaataggtcttgggataggagaaacaatcctattccaagagcttggaaattttgaaaatgataaagattacatagtaatagtggaaaaaagtttcagcttcttagaggtagctcacagaatcaatgaagcttttaaaaatggatgcaaaataaagggtgctattattcaaaaagatgatggggttctcataaataacagactcataaataaaatccccatagttgatgaggtactttttgttaaaaaagtacctacagggatgaaggctgctgtagaagtagctccacagggaaaaataatagaggttatttcaaatccatatggcattgccacaattttttccctcacttcagaagagactaaaaaaatagttcctatttctaaagcacttataggcaacaggtctggagtagttatcaagacacctcacggagatgtaaaagagaaggttatccctgctggaaggatacagattgacggaaactacaggtcaaaaagtgtaaatatagaagagggttccaaaagaataatgaaagccctgggaagtattgagcatgtccaagatataaatggagaatctggaaccaatatcggaggaatgctaaaaaatgtaaaaagtgtaatggggaatttcaccaatgagtccattgataatataaaaataaaagacatattggcagtagatacctttgtcccacaaaagataaaggggggaattgcagaagaatttgtatttgaaaatgctgtaggaatagctgcaatggtaaataccaaaaaaaatcaaatgtccgaagtagcgaaagagattgaaaaagaactgggagtaaaagtagaagtaggaggagtagaggcagatatggctataaccggtgctctaactactccaggcacaggaacacctctggtaattgtagatataggagcaggttcgacagatgcatgttccattgacagatatggaaataaagaactggttcatctggccggagctggtaatatgacaacacttcttattcaaaaagagctgggtatagaggattttaatcttgctgaagatataaaaaaatatcctctggcaaaagtagaatctctattttatataagacacgaggatggaaatgttcaattttttgaaaactctctttctccgaaagtatttgctaaaaatgtccttataaaagaaggtgaacttattccaatcgaccttgatatgtctctggaaaaaatcagaattatcagaaggtctgccaaaagaaaaatttttataaccaatgtacttagatcattaaggaaagtttctcatacaaaaaatattagggattttgaatttgtagttattgttggaggatctgcattggattttgaaatatctcagatgataactgaagctttatctgagtatggaatagtagcaggatgcggaaatataagaggaacagagggccctagaaatgctgtagccactggacttgtaatgggggtgaatgatggacaacaggcctaa

SEQ ID NO: 222 - DNA, 324 bp, Ilyobacter polytropus
  Gene (1)..(324): gdrB gene
atggacaacaggcctaatataacattattttgctcagataatattgacagggaatatattaatgaaattttgtggggtatagaggaggaagagataccatatcttctgaaaattgtaccttctaaagaagttgtcaaagaaaattatgtttcaggaactctagagataggtatcggagtattagaaaatggcgacgcccttctaacaacaaggaagtacgataaggaatatatacaaaaggcaaacatttttgtagaaaaaaataaattgagagatttaggaagcaacggagcaagacttgtaaagggtctgccacttagataa

SEQ ID NO: 223 - DNA, 1824 bp, Klebsiella pneumoniae
  Gene (1)..(1824): gdrA gene
atgccgttaatagccgggattgatatcggcaacgccaccaccgaggtggcgctggcgtccgacgacccgcaggcgagggcgtttgttgccagcgggatcgtcgcgacgacgggcatgaaagggacgcgggacaatatcgccgggaccctcgccgcgctggagcaggccctggcgaaaacaccgtggtcggtgagcgatgtctctcgcatctatcttaacgaagccgcgccggtgattggcgatgtggcgatggagaccatcaccgagaccattatcaccgaatcgaccatgatcggtcataacccgcagacgccgggcggggtgggcgttggcgtggggacgactatcgccctcgggcggctggcgacgctgccggcggcgcagtatgccgaggggtggatcgtactgattgacgacgccgtcgatttccttgacgccgtgtggtggctcaatgaggcgctcgaccgggggatcaacgtggtggcggcgatcctcaaaaaggacgacggcgtgctggtgaacaaccgcctgcgtaaaaccctgccggtggtagatgaagtgacgctgctggagcaggtccccgagggggtaatggcggcggtggaagtggccgcgccgggccaggtggtgcggatcctgtcgaatccctacgggatcgccaccttcttcgggctaagcccggaagagacccaggccatcgtccccatcgcccgcgccctgattggcaaccgttcagcggtggtgctcaagaccccgcagggggatgtgcagtcgcgggtgatcccggcgggcaacctctacattagcggcgaaaagcgccgcggagaggccgatgtcgccgagggcgcggaagccatcatgcaggcgatgagcgcctgcgctccggtacgcgacatccgcggcgaaccgggcactcacgccggcggcatgcttgagcgggtgcgcaaggtaatggcgtccctgaccgaccatgagatgagcgcgatatacatccaggatctgctggcggtggatacgtttattccgcgcaaggtgcagggcgggatggccggcgagtgcgccatggaaaatgccgtcgggatggcggcgatggtgaaagcggatcgtctgcaaatgcaggttatcgcccgcgaactgagcgcccgactgcagaccgaggtggtggtgggcggcgtggaggccaacatggccatcgccggggcgttaaccactcccggctgtgcggcgccgctggcgatcctcgacctcggcgccggctcgacggatgcggcgatcgtcaacgcggaggggcagataacggcggtccatctcgccggggcggggaatatggtcagcctgttgattaaaaccgagctgggcctcgaggatctttcgctggcggaagcgataaaaaaatacccgctggccaaagtggaaagcctgttcagtattcgtcacgagaatggcgcggtggagttctttcgggaagccctcagcccggcggtgttcgccaaagtggtgtacatcaaggagggcgaactggtgccgatcgataacgccagcccgctggaaaaaattcgtctcgtgcgccggcaggcgaaagagaaagtgtttgtcaccaactgcctgcgcgcgctgcgccaggtctcacccggcggttccattcgcgatatcgcctttgtggtgctggtgggcggctcatcgctggactttgagatcccgcagcttatcacggaagccttgtcgcactatggcgtggtcgccgggcagggcaatattcggggaacagaagggccgcgcaacgcggtcgccaccgggctgctactggccggtcaggcgaattaa

SEQ ID NO: 224 - DNA, 354 bp, Klebsiella pneumoniae
  Gene (1)..(354): gdrB gene
atgtcgctttcaccgccaggcgtacgcctgttttacgatccgcgcgggcaccatgccggcgccatcaatgagctgtgctgggggctggaggagcagggggtcccctgccagaccataacctatgacggaggcggtgacgccgctgcgctgggcgccctggcggccagaagctcgcccctgcgggtgggtattgggctcagcgcgtccggcgagatagccctcactcatgcccagctgccggcggacgcgccgctggctaccggacacgtcaccgatagcgacgatcatctgcgtacgctcggcgccaacgccgggcagctggttaaagtcctgccgttaagtgagagaaactga

SEQ ID NO: 225 - protein, 314 aa, Corynebacterium glutamicum
  MISC_FEATURE (1)..(314): LDH
MKETVGNKIVLIGAGDVGVAYAYALINQGMADHLAIIDIDEKKLEGNVMDLNHGVVWADSRTRVTKGTYADCEDAAMVVICAGAAQKPGETRLQLVDKNVKIMKSIVGDVMDSGFDGIFLVASNPVDILTYAVWKFSGLEWNRVIGSGTVLDSARFRYMLGELYEVAPSSVHAYIIGEHGDTELPVLSSATIAGVSLSRMLDKDPELEGRLEKIFEDTRDAAYHIIDAKGSTSYGIGMGLARITRAILQNQDVAVPVSALLHGEYGEEDIYIGTPAVVNRRGIRRVVELEITDHEMERFKHSANTLREIQKQFF

SEQ ID NO: 226 - DNA, 3267 bp, Artificial Sequence
  Synthetic pKD4 vector
agattgcagcattacacgtcttgagcgattgtgtaggctggagctgcttcgaagttcctatactttctagagaataggaacttcggaataggaacttcaagatcccctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccagcttcaaaagcgctctgaagttcctatactttctagagaataggaacttcggaataggaactaaggaggatattcatatggaccatggctaattcccatgtcagccgttaagtgttcctgtgtcactgaaaattgctttgagaggctctaagggcttctcagtgcgttacatccctggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgcggatcttgcggccgcaaaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgcatcgatggccccccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtggccagtgccaagcttgcatgc

SEQ ID NO: 227 - DNA, 58 bp, Artificial Sequence
  Synthetic ackAKF primer
cgtagtgatcgatgagtctgttattcagggtatcaaaggtgtaggctggagctgcttc

SEQ ID NO: 228 - DNA, 56 bp, Artificial Sequence
  Synthetic ackAKR primer
caatccctgcacccagttctacaccctgagacgctgattccggggatccgtcgacc

SEQ ID NO: 229 - DNA, 6329 bp, Artificial Sequence
  Synthetic pKD46 vector
catcgatttattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggaattcgagctctaaggaggttataaaaaatggatattaatactgaaactgagatcaagcaaaagcattcactaaccccctttcctgttttcctaatcagcccggcatttcgcgggcgatattttcacagctatttcaggagttcagccatgaacgcttattacattcaggatcgtcttgaggctcagagctgggcgcgtcactaccagcagctcgcccgtgaagagaaagaggcagaactggcagacgacatggaaaaaggcctgccccagcacctgtttgaatcgctatgcatcgatcatttgcaacgccacggggccagcaaaaaatccattacccgtgcgtttgatgacgatgttgagtttcaggagcgcatggcagaacacatccggtacatggttgaaaccattgctcaccaccaggttgatattgattcagaggtataaaacgaatgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatgacaccggacattatcctgcagcgtaccgggatcgatgtgagagctgtcgaacagggggatgatgcgtggcacaaattacggctcggcgtcatcaccgcttcagaagttcacaacgtgatagcaaaaccccgctccggaaagaagtggcctgacatgaaaatgtcctacttccacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcgatgacgcatcctcacgataatatccgggtaggcgcaatcactttcgtctactccgttacaaagcgaggctgggtatttcccggcctttctgttatccgaaatccactgaaagcacagcggctggctgaggagataaataataaacgaggggctgtatgcacaaagcatcttctgttgagttaagaacgagtatcgagatggcacatagccttgctcaaattggaatcaggtttgtgccaataccagtagaaacagacgaagaatccatgggtatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcggttgcggccgcaatcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 230 - DNA, 9332 bp, Artificial Sequence
  Synthetic pCP20 vector
gagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgccacaggtgcggttgctggcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgcttgtttcggcgtgggtatggtggcaggccccgtggccgggggactgttgggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgttcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagtatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcatttaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgcctgtttcgtttcaggcaggttatcagggagtgtcagcgtcctgcggttctccggggcgttcgggtcatgcagcccgtaatggtgatttaccagcgtctgccaggcatcaattctaggcctgtctgcgcggtcgtagtacggctggaggcgttttccggtctgtagctccatgttcggaatgacaaaattcagctcaagccgtcccttgtcctggtgctccacccacaggatgctgtactgatttttttcgagaccgggcatcagtacacgctcaaagctcgccatcactttttcacgtcctcccggcggcagctccttctccgcgaacgacagaacaccggacgtgtatttcttcgcaaatggcgtggcatcgatgagttcccggacttcttccggattaccctgaagcaccgttgcgccttcgcggttacgctccctccccagcaggtaatcaaccggaccactgccaccaccttttcccctggcatgaaatttaactatcatcccgcgccccctgttccctgacagccagacgcagccggcgcagctcatccccgatggccatcagtgcggccaccacctgaacccggtcaccggaagaccactgcccgctgttcaccttacgggctgtctgattcaggttatttccgatggcggccagctgacgcagtaacggcggtgccagtgtcggcagttttccggaacgggcaaccggctcccccaggcagacccgccgcatccataccgccagttgtttaccctcacagcgttcaagtaaccgggcatgttcatcatcagtaacccgtattgtgagcatcctctcgcgtttcatcggtatcattaccccatgaacagaaatcccccttacacggaggcatcagtgactaaacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattttataaaccgtggagcgggcaatactgagctgatgagcaatttccgttgcaccagtgcccttctgatgaagcgtcagcacgacgttcctgtccacggtacgcctgcggccaaatttgattcctttcagctttgcttcctgtcggccctcattcgtgcgctctaggatcctctacgccggacgcatcgtggccggcatcaccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatatgcatagtaccgagaaactagtgcgaagtagtgatcaggtattgctgttatctgatgagtatacgttgtcctggccacggcagaagcacgcttatcgctccaatttcccacaacattagtcaactccgttaggcccttcattgaaagaaatgaggtcatcaaatgtcttccaatgtgagattttgggccattttttatagcaaagattgaataaggcgcatttttcttcaaagctttattgtacgatctgactaagttatcttttaataattggtattcctgtttattgcttgaagaattgccggtcctatttactcgttttaggactggttcagaattcctcaaaaattcatccaaatatacaagtggatcgatcctaccccttgcgctaaagaagtatatgtgcctactaacgcttgtctttgtctctgtcactaaacactggattattactcccagatacttattttggactaatttaaatgatttcggatcaacgttcttaatatcgctgaatcttccacaattgatgaaagtagctaggaagaggaattggtataaagtttttgtttttgtaaatctcgaagtatactcaaacgaatttagtattttctcagtgatctcccagatgctttcaccctcacttagaagtgctttaagcatttttttactgtggctatttcccttatctgcttcttccgatgattcgaactgtaattgcaaactacttacaatatcagtgatatcagattgatgtttttgtccatagtaaggaataattgtaaattcccaagcaggaatcaatttctttaatgaggcttccagaattgttgctttttgcgtcttgtatttaaactggagtgatttattgacaatatcgaaactcagcgaattgcttatgatagtattatagctcatgaatgtggctctcttgattgctgttccgttatgtgtaatcatccaacataaataggttagttcagcagcacataatgctattttctcacctgaaggtctttcaaacctttccacaaactgacgaacaagcaccttaggtggtgttttacataatatatcaaattgtggcatacaacctccttagtacatgcaaccattatcaccgccagaggtaaaatagtcaacacgcacggtgttagatatttatcccttgcggtgatagatttaacgtatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctaagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctgatcggcaaggtgttctggtcggcgcatagctgataacaattgagcaagaatctgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagatttt

SEQ ID NO: 231 - DNA, 65 bp, Artificial Sequence
  Synthetic yqhDKF primer
cgccatcatggcggtgcggcgctgccttccagttcggttaacacggtgtaggctggagctgcttc

SEQ ID NO: 232 - DNA, 65 bp, Artificial Sequence
  Synthetic yqhDKR primer
gcgcgagttctcaataatggcgcgtttggtgcgaacttcgtggtaattccggggatccgtcgacc

SEQ ID NO: 233 - DNA, 32 bp, Artificial Sequence
  Synthetic dhaB123_F primer
tcatgaaatcaaaaagatttgaagtattgaag

SEQ ID NO: 234 - DNA, 36 bp, Artificial Sequence
  Synthetic dhaB123_R primer
ggatccctaatcttttctaagttgacctctttgttc

SEQ ID NO: 235 - DNA, 30 bp, Artificial Sequence
  Synthetic gdrAB_F primer
ggatccaaaggttcggggatagttatgaag

SEQ ID NO: 236 - DNA, 33 bp, Artificial Sequence
  Synthetic gdrAB_R primer
gagctcttatctaagtggcagaccctttacaag

SEQ ID NO: 237 - DNA, 30 bp, Artificial Sequence
  Synthetic iBAB_Up primer
atgtatatctccttcttatacttaactaat

SEQ ID NO: 238 - DNA, 35 bp, Artificial Sequence
  Synthetic iBAB_Dn primer
atcggccggccacgcgatcgctgacgtcggtaccc

SEQ ID NO: 239 - DNA, 35 bp, Artificial Sequence
  Synthetic pduP_F primer
gaaggagatatacatatgcagattaatgatattga

SEQ ID NO: 240 - DNA, 35 bp, Artificial Sequence
  Synthetic pduP_R primer
gcgtggccggccgatttaataccagttacgtactg

SEQ ID NO: 241 - DNA, 49 bp, Artificial Sequence
  Synthetic primer for MELS_1449 gene
tcatcaccacagccaggatccgatggcactaagagatgggaattcctac

SEQ ID NO: 242 - DNA, 43 bp, Artificial Sequence
  Synthetic primer for MELS_1449 gene
gcattatgcggccgcaagcttttatttttcttcctttatgccg

SEQ ID NO: 243 - DNA, 43 bp, Artificial Sequence
  Synthetic yciA_F primer
tcatcaccacagccaagatctgatgtctacaacacataacgtc

SEQ ID NO: 244 - DNA, 40 bp, Artificial Sequence
  Synthetic yciA_R primer
gcattatgcggccgcctcgagttactcaacaggtaaggcg

(19) United States
(12) Patent Application Publication
(10) Pub. No.: US 2016/0010124 A1
(43) Pub. Date: Jan. 14, 2016

(54) MICROORGANISM HAVING NOVEL ACRYLIC ACID SYNTHESIS PATHWAY HAVING ENHANCED ACTIVITY OF COA ACYLATING ALDEHYDE DEHYDROGENASE AND METHOD OF PRODUCING ACRYLIC ACID USING THE SAME

(71) Applicant: Samsung Electronics Co., Ltd., Suwon-si (KR)

(72) Inventors: Taewook Nam, Seoul (KR); Yeoju Song, Incheon (KR); Seunghoon Song, Yongin-si, Gyeonggi-do (KR); Jiae Yun, Hwaseong-si (KR); Jinsuk Lee, Seongnam-si (KR); Hunsu Chu, Seoul (KR); Insuk Choi, Seongnam-si (KR)

(21) Appl. No.: 14/620,002

(22) Filed: Feb. 11, 2015

(30) Foreign Application Priority Data
Jul. 8, 2014 (KR) 10-2014-0085356

(51) Int. Cl. C12P 7/62; C12N 15/70; C12N 9/24; C12N 9/02; C12N 9/88

(52) U.S. Cl. CPC C12P 7/62; C12N 9/0008; C12N 9/88; C12N 9/2402; C12N 15/70; C12Y 102/0101; C12Y 402/01

(57) ABSTRACT

A microorganism capable of producing acrylic acid, comprising a genetic modification that increases activity of CoA acylating aldehyde dehydrogenase (ALDH) catalyzing conversion of 3-hydroxypropionaldehyde (3-HPA) to 3-hydroxy propionyl-CoA (3-HP-CoA) and a genetic modification that increases activity of 3-HP-CoA dehydratase catalyzing conversion of 3-HP-CoA to acrylyl-CoA in the microorganism in comparison with a cell that is not genetically engineered; as well as a method of producing the microorganism, and a method of producing acrylic acid using the same.

### RELATED APPLICATION

This application claims the benefit of Korean Patent Application No. 10-2014-0085356, filed on Jul. 8, 2014, in the Korean Intellectual Property Office, the entire disclosure of which is hereby incorporated by reference.

### INCORPORATION BY REFERENCE OF ELECTRONICALLY SUBMITTED MATERIALS

Incorporated by reference in its entirety herein is a computer-readable nucleotide/amino acid sequence listing submitted herewith and identified as follows: One 531,423 bytes ASCII (Text) file named “719113_ST25.TXT” created Feb. 10, 2015.

### BACKGROUND

1. Field

The present disclosure relates to a microorganism having a novel acrylic acid synthesis pathway and a method of producing acrylic acid using the same.

2. Description of the Related Art

As the instability caused by the recent rise of oil prices and pressure to reduce carbon emissions become global issues, efforts are continuously made to replace conventional petroleum-based chemical processes for producing fuels or chemicals with carbon neutral biological processes.

Acrylic acid is a bulk chemical having an annual market size of 10 trillion Korean Won (KRW). Recently, there has been an increasing need for a method of producing acrylic acid through a pathway besides a petroleum-based pathway due to the requirement for an environment-friendly production method.

A non-petroleum acrylic acid production pathway may include producing 3-hydroxypropionate (3-HP) from glycerol or glucose, and then chemically separating and purifying 3-HP. However, this method includes separating and purifying the produced 3-HP from a culture medium and chemically converting by using a catalyst. Therefore, the cost for the separation, purification, and conversion is added to the 3-HP production cost, and thus the competitiveness of the method may not be high in comparison with a petroleum compound-derived acrylic acid production method.

Even when a conventional technology is used, there is a need for an alternative microorganism capable of producing acrylic acid and a method of producing acrylic acid using the same.

### SUMMARY

An aspect of the present disclosure provides a microorganism having an increased capability of producing acrylate in comparison with a cell that is not genetically engineered.

Another aspect of the present disclosure provides a method of producing acrylate including culturing of the microorganism in a culture medium.

### BRIEF DESCRIPTION OF THE DRAWINGS

These and/or other aspects will become apparent and more readily appreciated from the following description of several embodiments, taken in conjunction with the accompanying drawings in which:

FIG. 1 is a cleavage map of pET-iBAB_PduP vector;

FIG. 2 is a graph showing the HPLC analysis results of acrylate in a culture solution when two recombinant E. coli strains, which were made by introducing ALDH and 3-HP-CoA dehydratase genes into E. coli SH3, were cultured in a glycerol-containing medium for 48 hours;

FIG. 3 is a graph showing the amount of acrylate in a culture solution after culturing an E. coli SH3/pET-iBAB-PduP/pACYC-MDH strain in a fermenter for 48 hours; and

FIG. 4 is a diagram showing an expected pathway of producing acrylic acid from glucose or glycerol in E. coli according to Example 1.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

The term “activity increase” or “increased activity” and the like in reference to a cell, an enzyme, a polypeptide, or a protein used herein may refer to any detectable increase in activity sufficient to show that the activity level of the cell, enzyme, polypeptide, or protein is higher than that of a comparable cell, enzyme, polypeptide or protein (e.g., a cell, polypeptide, protein or enzyme of the same type that is not genetically engineered). For instance, the activity may be increased by about 5%, about 10%, about 15%, about 20%, about 30%, about 50%, about 60%, about 70%, about 100%, about 200%, or about 300% in comparison with the same biological activity a cell, polypeptide, protein, or enzyme which is not genetically engineered. Increased activity may be verified by using a method known to those of ordinary skill in the art.

The activity increase of a polypeptide, protein, or enzyme may be achieved by, for example, expression increase or increase of specific activity of a polypeptide, protein, or enzyme (hereinafter referred to collectively as “polypeptide”). The expression increase may be caused by introduction of a polynucleotide encoding the polypeptide into a cell, by increase of the number of copies of a polynucleotide encoding a polypeptide in a cell, or by mutation of a regulatory region of a polynucleotide encoding the polypeptide. A polynucleotide which is introduced into the cell, or whose copy number is increased, may be endogenous or exogenous. “Exogenous” as it is used herein is intended to mean that the referenced molecule or the referenced activity is introduced into the host microbial organism. The molecule can be introduced, for example, by introduction of an encoding nucleic acid into the host genetic material so that it integrates into a host chromosome or in a form that remains as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host before genetic manipulation. Similarly, the term when used in reference to expression of an encoding nucleic acid refers to expression of an encoding nucleic acid contained within the microbial organism. When used in reference to a source, the term “heterologous” refers to a molecule or activity derived from a source other than the referenced species whereas “homologous” refers to a molecule or activity derived from the host microbial organism itself. Accordingly, expression of an exogenous encoding nucleic acid can utilize either or both a heterologous or homologous encoding nucleic acid.

The term “copy number increase” may be an increase of copy number by the introduction of an exogenous gene into a host cell, or amplification of an endogenous gene, and, thus, includes causing by genetic engineering a cell to have a gene which is not preexisting in the cell. The introduction of a gene may be mediated by a vehicle such as a vector. The introduction may be a transient introduction in which the gene is not integrated to a genome or insertion of the gene into a genome. The introduction may be performed, for example, by introducing into the cell a vector to which a polynucleotide encoding a target polypeptide is inserted, and then replicating the vector in the cell or integrating the polynucleotide into the genome.

As used herein, the term “genetic modification” may refer to introduction of a polynucleotide encoding a polypeptide (i.e., an increase in a copy number of the gene), or substitution, addition, insertion, or deletion of at least one nucleotide with a genetic material of a parent cell, or chemical mutation of a genetic material of a parent cell. In other words, genetic modification may include cases associated with a coding region of a polypeptide or a functional fragment thereof of a polypeptide that is heterologous, homologous, or both heterologous and homologous with a referenced species. Genetic modification may also refer to modification in non-coding regulatory regions that are capable of modifying expression of a gene or an operon, wherein the non-coding regulatory regions include a 5′-non coding sequence and/or a 3′-non coding sequence.

The term “gene” refers to a nucleic acid fragment expressing a specific protein and may include a coding region as well as regulatory sequences such as a 5′-non coding sequence or a 3′-non coding sequence. The regulatory sequences may include a promoter, an enhancer, an operator, a ribosome binding site, a polyA binding site, and a terminator region.

The term “secretion” means transport of a material from the inside of a cell to a periplasmic space or an extracellular environment.

The term “cell,” “strain,” or “microorganism” may be interchangeably used and includes bacterial, yeasts, and fungi.

The term “acrylic acid” includes acrylic acid or acrylate, or a salt thereof, which may be used interchangeably. Acrylic acid may be produced by fermentation or an enzymatic reaction of a microorganism.

The term “activity decrease” or “decreased activity” or “reduced activity” and the like in reference to a cell, an enzyme or a polypeptide (including an enzyme or protein) used herein mean that the activity level of a cell or polypeptide is lower than an activity level measured in the same kind of comparable cell or the original polypeptide, or shows no activity. For instance, the term may refer to an activity of a cell or polypeptide which is decreased by about 10%, about 20%, about 30% or more, about 40% or more, about 50% or more, about 55% or more, about 60% or more, about 70% or more, about 75% or more, about 80% or more, about 85% or more, about 90% or more, about 95% or more, or about 100% in comparison with the same biological activity of the original cell or polypeptide which is not genetically engineered. A polypeptide having a decreased activity may be verified by using a method known to those of ordinary skill in the art. The activity decrease includes the case where an enzyme is expressed but the enzyme activity is not detectable or is decreased, and the case where a gene encoding an enzyme is not expressed or, even when the gene is expressed, the expression is lower than the expression of a gene that is not genetically engineered.

Decreased activity of a polypeptide (including an enzyme or protein) may be caused by a deletion or disruption of a gene encoding the polypeptide. The term “deletion” or “disruption” used herein refers to mutation, substitution, or deletion of a part of or the whole gene or a part of or the whole regulatory region such as a promoter or a terminator of a gene, or insertion of at least one base group to a gene for preventing a gene's expression or for preventing an expressed polypeptide from showing activity or making an expressed enzyme show a decreased activity level. The deletion or disruption of the gene may be achieved by gene manipulation such as homogenous recombination, mutation generation, or molecule evolution. When a cell includes a plurality of the same genes or at least two different polypeptide paralogous genes, one or more genes may be deleted or disrupted.

The term “sequence identity” of a nucleic acid or a polypeptide used herein refers to a degree of similarity of base groups or amino acid residues between two aligned sequences, when the two sequences are aligned to match each other as possible (i.e., to an optimum state), at corresponding positions. The sequence identity is a value that is measured by aligning to an optimum state and comparing the two sequences at a particular comparing region, wherein a part of the sequence within the particular comparing region may be added or deleted compared to a reference sequence. A sequence identity percentage may be calculated, for example, by comparing the two sequences aligned within the whole comparing region to an optimum; obtaining the number of matched locations by determining the number of locations represented by the same amino acids of nucleic acids in both of the sequences; dividing the number of the matched locations by the total number of the locations within the comparing region (i.e., a range size); and obtaining a percentage of the sequence identity by multiplying 100 to the result. The sequence identity percent may be determined by using a common sequence comparing program, for example, BLASTN (NCBI), CLC Main Workbench (CLC bio), MegAlign™ (DNASTAR Inc).

In confirming many different polypeptides or polynucleotides having the same or similar function or activity, sequence identities at several levels may be used. For example, the sequence identities may include about 50% or greater, about 55% or greater, about 60% or greater, about 65% or greater, about 70% or greater, about 75% or greater, about 80% or greater, about 85% or greater, about 90% or greater, about 95% or greater, about 96% or greater, about 97% or greater, about 98% or greater, about 99% or greater, or 100%.

An aspect of the present disclosure provides a microorganism having capability of producing acrylate, wherein activity of CoA acylating aldehyde dehydrogenase (ALDH) catalyzing conversion of 3-hydroxypropionaldehyde (3-HPA) to 3-hydroxy propionyl-CoA (3-HP-CoA) and activity of 3-HP-CoA dehydratase catalyzing conversion of 3-HP-CoA to acrylyl-CoA are increased in the microorganism in comparison with a cell that is not genetically engineered.

The ALDH may belong to EC 1.2.1.10 or EC 1.2.1.87. The ALDH has a higher activity of catalyzing conversion of 3-HPA to 3-HP-CoA than the activity of catalyzing the reverse reaction. The ALDH may include an amino acid sequence having about 65% or more, for example, 70% or more, about 80% or more, about 85% or more, about 90% or more, about 91% or more, about 92% or more, about 93% or more, about 94% or more, about 95% or more, about 96% or more, about 97% or more, about 98% or more, about 99% or more, or about 100% sequence identity with amino acid sequences of SEQ ID NOS: 1 to 20. A polynucleotide encoding the ALDH may encode an amino acid sequence having about 65% or more, for example, 70% or more, about 80% or more, about 85% or more, about 90% or more, about 91% or more, about 92% or more, about 93% or more, about 94% or more, about 95% or more, about 96% or more, about 97% or more, about 98% or more, about 99% or more, or about 100% sequence identity with amino acid sequences of SEQ ID NOS: 1 to 20. A polynucleotide encoding the ALDH may have about 95% or more sequence identity with nucleotide sequences of SEQ ID NOS: 21 to 40. The ALDH may be at least one of the enzymes shown in Tables 1 and 2. The ALDH may catalyze a reaction described below, regardless of the name. The ALDH may be CoA-acylating propionaldehyde dehydrogenase, aldehyde dehydrogenase, alcohol dehydrogenase, CoA-dependent aldehyde dehydrogenase, or a combination thereof. The ALDH may be pduP, for example, Lactobacillus reuteri-derived pduP.

3-HPA+CoA+NAD(P)+->3-HP-CoA+NAD(P)H

TABLE 1

| | | | | Gene | Purchased | |
|---|---|---|---|---|---|---|
| NO. | EC | Category | Source Strain | Name | from | Sequence* |
| 1 | 1.2.1.10 | 50S ribosomal protein L29 | Lactobacillus reuteri | Lreu_1735 | KCTC 3594 | 1/21 |
| | | | DSM 20016 | | | |
| 2 | 1.2.1.10 | CoA-dependent propionaldehyde | Lactobacillus brevis | LVIS_1603 | ATCC 367 | 2/22 |
| | | dehydrogenase | ATCC 367 | | | |
| 3 | 1.2.1.10 | aldehyde dehydrogenase | Pediococcus acidilactici | HMPREF | KCTC 1626 | 3/23 |
| | | | | 9024_01049 | | |
| 4 | 1.2.1.10 | CoA-dependent propionaldehyde | Pediococcus claussenii | pduP | DSM 14800 | 4/24 |
| | | dehydrogenase | ATCC BAA-344 | | | |
| 5 | 1.2.1.10 | PduP protein | Lactobacillus | pduP | KCTC 5050 | 5/25 |
| | | | collinoides | | | |
| 6 | 1.2.1.10 | CoA-dependent propionaldehyde | Listeria welshimeri | NC_008555.1: | ATCC 35897 | 6/26 |
| | | dehydrogenase | serovar 6b str. | 1134599 . . . 1136008 | | |
| | | | SLCC5334 | | | |
| 7 | 1.2.1.10 | hypothetical protein lin1129 | Listeria innocua | NC_003212.1: | ATCC 33090 | 7/27 |
| | | | Clip11262 | 1144168 . . . 1145577 | | |
| 8 | 1.2.1.10 | propanediol utilization Co-A | Listeria monocytogenes | pduP | ATCC 19117 | 8/28 |
| | | dependent propionaldehyde | ATCC 19117 | | | |
| | | dehydrogenase | | | | |
| 9 | 1.2.1.10 | ethanolamine utilization | Listeria marthii | NT05LM_1376 | ATCC BAA-1595 | 9/29 |
| | | protein EutE | FSL S4-120 | | | |
| 10 | 1.2.1.10 | putative ethanolamine | Listeria ivanovii | LIV_1097 | ATCC BAA-678 | 10/30 |
| | | utilization protein EutE | subsp. ivanovii | | | |
| | | | PAM 55 | | | |

*The sequence represents an amino acid SEQ ID NO/a nucleotide SEQ ID NO.

TABLE 2

| | | | | | | |
|---|---|---|---|---|---|---|
| 11 | 1.2.1.10 | CoA-dependent propionaldehyde | Listeria seeligeri | pduP | ATCC 35967 | 11/31 |
| | | dehydrogenase | serovar 1/2b str. | | | |
| | | | SLCC3954 | | | |
| 12 | 1.2.1.10 | aldehyde dehydrogenase | Shewanella putrefaciens | NC_009438.1: | ATCC BAA-453 | 12/32 |
| | | | CN-32 | 221466 . . . 222860 | | |
| 13 | 1.2.1.10 | aldehyde dehydrogenase family | Kosakonia radicincitans | Y71_5889 | DSM 16656 | 13/33 |
| | | protein | DSM 16656 | | | |
| 14 | 1.2.1.10 | Aldehyde Dehydrogenase | Tolumonas auensis | NC_012691.1: | DSM 9187 | 14/34 |
| | | | DSM 9187 | 1861535 . . . 1862938 | | |
| 15 | 1.2.1.10 | hypothetical protein CKO_00785 | Citrobacter koseri | NC_009792.1: | ATCC BAA-895 | 15/35 |
| | | | ATCC BAA-895 | 757825 . . . 759210 | | |
| 16 | 1.2.1.10 | propanediol utilization CoA- | Yersinia enterocolitica | NC_008800.1: | ATCC 9610 | 16/36 |
| | | dependent propionaldehyde | subsp. enterocolitica | 2975153 . . . 2976541 | | |
| | | dehydrogenase | 8081 | | | |
| 17 | 1.2.1.10 | aldehyde dehydrogenase EutE | Salmonella enterica | SEEM1958_22984 | ATCC 51958 | 17/37 |
| | | | subsp. enterica | | | |
| | | | serovar Mbandaka | | | |
| | | | str. ATCC 51958 | | | |
| 18 | 1.2.1.10 | putative propanediol utilization | Yersinia mollaretii | ymoll0001_15900 | ATCC 43969 | 18/38 |
| | | protein: CoA-dependent | ATCC 43969 | | | |
| | | propionaldehyde dehydrogenase | | | | |
| 19 | 1.2.1.10 | CoA-dependent proprionaldehyde | Escherichia fergusonii | NC_011740.1: | ATCC 35469 | 19/39 |
| | | dehydrogenase pduP | ATCC 35469 | 2070780 . . . 2072162 | | |
| 20 | 1.2.1.10 | putative CoA-dependent | Salmonella enterica | eutE | ATCC 9261 | 20/40 |
| | | proprionaldehyde dehydrogenase | subsp. enterica | | | |
| | | | serovar Urbana str. | | | |
| | | | ATCC 9261 | | | |

The 3-HP-CoA dehydratase may belong to EC 4.2.1. including EC 4.2.1.17, EC 4.2.1.55, and EC 4.2.1.166. The 3-HP-CoA dehydratase may have a higher activity of catalyzing conversion of 3-HP-CoA to acrylyl-CoA than the activity of catalyzing the reverse reaction. The 3-HP-CoA dehydratase may include an amino acid sequence having about 65% or more, for example, 70% or more, about 80% or more, about 85% or more, about 90% or more, about 91% or more, about 92% or more, about 93% or more, about 94% or more, about 95% or more, about 96% or more, about 97% or more, about 98% or more, about 99% or more, or about 100% sequence identity with amino acid sequences of SEQ ID NOS: 41 to 119. A polynucleotide encoding the 3-HP-CoA dehydratase may encode an amino acid sequence having about 65% or more, for example, 70% or more, about 80% or more, about 85% or more, about 90% or more, about 91% or more, about 92% or more, about 93% or more, about 94% or more, about 95% or more, about 96% or more, about 97% or more, about 98% or more, about 99% or more, or about 100% sequence identity with amino acid sequences of SEQ ID NOS: 41 to 119. A polynucleotide encoding the 3-HP-CoA dehydratase may have about 95% or more sequence identity with nucleotide sequences of SEQ ID NOS: 120 to 198. The 3-HP-CoA dehydratase may be at least one of enzymes shown in Tables 3 to 6. The enzymes shown in Tables 3 to 6 may be an E2 type. In Tables 3 to 6, the “Sequence*” refers to an amino acid/nucleotide SEQ ID NO.

TABLE 3

| | | | | | Purchased | |
|---|---|---|---|---|---|---|
| NO | EC | Category | Source Strain | Gene Name | from | Sequence* |
| 1 | 4.2.1.— | 3-hydroxybutyryl-CoA | Dictyostelium | Q869N6 | DSM947 | 41/120 |
| | | dehydratase(Crotonase) | discoideum | | | |
| | | | (Slime mold) | | | |
| 2 | 4.2.1.55 | 3-hydroxybutyryl-CoA | Clostridium | crt | KCTC1790 | 42/121 |
| | | dehydratase(Crotonase) | acetobutylicum | CA_C2712 | | |
| 3 | 4.2.1.55 | 3-hydroxybutyryl-CoA | Clostridium difficile | crt ech | KCTC5009 | 43/122 |
| | | dehydratase(Crotonase) | | | | |
| 4 | 4.2.1.55 | 3-hydroxybutyryl-CoA | Clostridium | F502_09038 | KCTC1674 | 44/123 |
| | | dehydratase(Crotonase) | pasteurianum | | | |
| 5 | 4.2.1.55 | 3-hydroxybutyryl-CoA | Clostridium | F502_06297 | KCTC1674 | 45/124 |
| | | dehydratase(Crotonase) | pasteurianum | | | |
| 6 | 4.2.1.55 | 3-hydroxybutyryl-CoA | Megasphaera elsdenii | MELS_1449 | KCTC5187 | 46/125 |
| | | dehydratase(Crotonase) | | | | |
| 7 | 4.2.1.116 | 3-hydroxybutyryl-CoA | Metallosphaera sedula | Msed_2001 | DSM5348 | 47/126 |
| | | dehydratase(Crotonase) | | | | |
| 8 | 4.2.1.55 | 3-hydroxybutyryl-CoA | Clostridicum kluyvery | crt1 | DSM555 | 48/127 |
| | | dehydratase(Crotonase) | | | | |
| 9 | 4.2.1.— | 4-hydroxybutyryl-CoA | Sulfolobus tokodaii | STK_16590 | DSM16993 | 49/128 |
| | | dehydratase | | | | |
| 10 | 4.2.1.— | 4-hydroxybutyryl-CoA | Geobacter | Gmet_2215 | DSM7210 | 50/129 |
| | | dehydratase | metallireducens | | | |
| 11 | 4.2.1.— | 4-hydroxybutyryl-CoA | Sulfolobus solfataricus | abfD-1 | DSM1617 | 51/130 |
| | | dehydratase | | | | |
| 12 | 4.2.1.— | 4-hydroxybutyryl-CoA | Syntrophobacter | Sfum_3141 | DSM10017 | 52/131 |
| | | dehydratase | fumaroxidans | | | |
| 13 | 4.2.1.— | 4-hydroxybutyryl-CoA | Porphyromonas | PGN_0727 | DSM20709 | 53/132 |
| | | dehydratase | gingivalis | | | |
| 14 | 4.2.1.— | 4-hydroxybutyryl-CoA | Polynucleobacter | Pnuc_0370 | DSM18221 | 54/133 |
| | | dehydratase | necessarius | | | |
| | | | subsp. Asymbioticus | | | |
| 15 | 4.2.1.116 | 3-hydroxypropionyl-CoA | Sulfolobus tokodaii | STK_15160 | DSM16993 | 55/134 |
| | | dehydratase | | | | |
| 16 | 4.2.1.— | 3-hydroxypropionyl-CoA | Gordonia terrae C-6 | GTC6_11571 | KCTC9807 | 56/135 |
| | | dehydratase | | | | |
| 17 | 4.2.1.— | 3-hydroxypropionyl-CoA | Halalkalicoccus jeotgali | HacjBS_17558 | DSM18796 | 57/136 |
| | | dehydratase | | C497_07209 | | |
| 18 | 4.2.1.— | 3-hydroxypropionyl-CoA | Carboxydothermus | CHY_1739 | DSM6008 | 58/137 |
| | | dehydratase | hydrogenoformans | | | |
| 19 | 4.2.1.55 | 3-hydroxypropionyl-CoA | Thermomicrobium | trd_0041 | DSM5159 | 59/138 |
| | | dehydratase | roseum | | | |
| 20 | 4.2.1.17 | 3-hydroxypropionyl-CoA | Methylobacterium | croA | DSM1337 | 60/139 |
| | | dehydratase | extorquens | METDI5699 | | |

TABLE 4

| | | | | | Purchased | |
|---|---|---|---|---|---|---|
| NO. | EC | Category | Source Strain | Gene Name | from | Sequence* |
| 21 | 4.2.1.— | R-phenyllactate | Clostridium | fldB | KCTC5654 | 61/140 |
| | | dehydratase | sporogenes | | | |
| 22 | 4.2.1.— | R-phenyllactate | | fldC | KCTC5654 | 62/141 |
| | | dehydratase | | | | |
| 23 | 4.2.1.— | R-phenyllactate | | fldI | KCTC5654 | 63/142 |
| | | dehydratase | | | | |
| 24 | 4.2.1.— | R-phenyllactate | | fldA | KCTC5654 | 64/143 |
| | | dehydratase | | | | |
| 25 | 4.2.1.— | R-phenyllactate | Lachnoanaerobaculum | fldC | DSM3986 | 65/144 |
| | | dehydratase | saburreum | HMPREF0381_2734 | | |
| 26 | 4.2.1.— | R-phenyllactate | | fldB | DSM3986 | 66/145 |
| | | dehydratase | | HMPREF0381_2735 | | |
| 27 | 4.2.1.— | R-phenyllactate | | fldI2 | DSM3986 | 67/146 |
| | | dehydratase | | HMPREF0381_2736 | | |
| 28 | 4.2.1.— | R-phenyllactate | Peptostreptococcus | fldI | DSM17678 | 68/147 |
| | | dehydratase | stomatis | HMPREF0634_1391 | | |
| 29 | 4.2.1.— | R-phenyllactate | | HMPREF0634_1028 | DSM17678 | 69/148 |
| | | dehydratase | | | | |
| 30 | 4.2.1.— | R-phenyllactate | | fldB | DSM17678 | 70/149 |
| | | dehydratase | | HMPREF0634_1029 | | |
| 31 | 4.2.1.— | 2-hydroxyisocaproyl-CoA | Clostridium | hadB | KCTC5009 | 71/150 |
| | | dehydratase | difficile | | | |
| 32 | 4.2.1.— | 2-hydroxyisocaproyl-CoA | | hadC | KCTC5009 | 72/151 |
| | | dehydratase | | | | |
| 33 | 4.2.1.— | 2-hydroxyisocaproyl-CoA | | hadI | KCTC5009 | 73/152 |
| | | dehydratase | | | | |
| 34 | 4.2.1.— | 2-hydroxyisocaproyl-CoA | | hadA | KCTC5009 | 74/153 |
| | | dehydratase | | | | |
| 35 | 4.2.1.17 | Enoyl-CoA hydratase | Escherichia coli | paaF | Possessed by | 75/154 |
| | | | (strain K12) | | Inventors | |
| 36 | 4.2.1.17 | Enoyl-CoA hydratase | Rhodobacter | fadB1 | KCTC2583 | 76/155 |
| | | | capsulatus | | | |
| 37 | 4.2.1.— | Enoyl-CoA hydratase | Pseudomonas | PSTAA_0117 | DSM4166 | 77/156 |
| | | | stutzeri | | | |
| 38 | 4.2.1.— | Enoyl-CoA hydratase | Haliangium | Hoch_4602 | DSM14365 | 78/157 |
| | | | ochraceum | | | |
| 39 | 4.2.1.— | Enoyl-CoA hydratase | Anoxybacillus | Aflv_0566 | DSM21510 | 79/158 |
| | | | flavithermus | | | |
| 40 | 4.2.1.— | Enoyl-CoA hydratase | Streptomyces | echA3 SAV_717 | DSM46492 | 80/159 |
| | | | avermitilis | | | |
| 41 | 4.2.1.— | Enoyl-CoA hydratase | Advenella | TKWG_10020 | DSM17095 | 81/160 |
| | | | kashmirensis | | | |

TABLE 5

| | | | | | Purchased | |
|---|---|---|---|---|---|---|
| NO. | EC | Category | Source Strain | Gene Name | from | Sequence* |
| 42 | 4.2.1.— | Enoyl-CoA hydratase | Oligotropha | OCA5_C12950 | DSM1227 | 82/161 |
| | | | carboxidovorans | OCAR_6780 | | |
| 43 | 4.2.1.— | Enoyl-CoA hydratase | Riemerella | Riean_1526 | DSM15868 | 83/162 |
| | | | anatipestifer | RA0C_1812 | | |
| 44 | 4.2.1.— | Enoyl-CoA hydratase | Fusobacterium | HMPREF1127_1435 | DSM19678 | 84/163 |
| | | | necrophorum | | | |
| | | | subsp. funduliforme | | | |
| | | | Fnf 1007 | | | |
| 45 | 4.2.1.— | Enoyl-CoA hydratase | | HMPREF1127_1434 | DSM19678 | 85/164 |
| 46 | 4.2.1.— | Enoyl-CoA hydratase | | HMPREF1127_1436 | DSM19678 | 86/165 |
| 47 | 4.2.1.— | Enoyl-CoA hydratase | Desulfosporosinus | DesyoDRAFT_3696 | DSM17734 | 87/166 |
| | | | youngiae | | | |
| | | | DSM 17734 | | | |
| 48 | 4.2.1.— | Enoyl-CoA hydratase | | DesyoDRAFT_3695 | DSM17734 | 88/167 |
| 49 | 4.2.1.— | Enoyl-CoA hydratase | | DesyoDRAFT_3697 | DSM17734 | 89/168 |
| 50 | 4.2.1.— | Enoyl-CoA hydratase | Peptoniphilus | fldB | KCTC15023 | 90/169 |
| | | | indolicus | HMPREF9129_0353 | | |
| | | | ATCC 29427 | | | |
| 51 | 4.2.1.— | Enoyl-CoA hydratase | | HMPREF9129_0354 | KCTC15023 | 91/170 |
| 52 | 4.2.1.— | Enoyl-CoA hydratase | | HMPREF9129_0352 | KCTC1502 | 92/171 |
| 53 | 4.2.1.— | Enoyl-CoA hydratase | Desulfosporosinus | Desmer_1800 | DSM13257 | 93/172 |
| | | | meridiei | | | |
| | | | (strain ATCC BAA-275/ | | | |
| | | | DSM 13257/NCIMB | | | |
| | | | 13706/S10) | | | |
| 54 | 4.2.1.— | Enoyl-CoA hydratase | | Desmer_1801 | DSM13257 | 94/173 |
| 55 | 4.2.1.— | Enoyl-CoA hydratase | | Desmer_1799 | DSM13257 | 95/174 |
| 56 | 4.2.1.— | 2-hydroxyglutaryl-CoA | Acidaminococcus | hgdA | DSM20731 | 96/175 |
| | | dehydratase | fermentans | Acfer_1815 | | |
| 57 | 4.2.1.— | 2-hydroxyglutaryl-CoA | | hgdB | DSM20731 | 97/176 |
| | | dehydratase | | Acfer_1815 | | |
| 58 | 4.2.1.— | 2-hydroxyglutaryl-CoA | | hgdC | DSM20731 | 98/177 |
| | | dehydratase | | Acfer_1815 | | |
| 59 | 4.2.1.— | 2-hydroxyglutaryl-CoA | Carboxydothermus | hgdB | DSM6008 | 99/178 |
| | | dehydratase | hydrogenoformans | CHY_0846 | | |
| 60 | 4.2.1.— | 2-hydroxyglutaryl-CoA | | hgdA | DSM6008 | 100/179 |
| | | dehydratase | | CHY_0847 | | |
| 61 | 4.2.1.— | 2-hydroxyglutaryl-CoA | | hgdC | DSM6008 | 101/180 |
| | | dehydratase | | CHY_0848 | | |
| 62 | 4.2.1.— | 2-hydroxyglutaryl-CoA | Oscillibacter | hgdC | DSM18026 | 102/181 |
| | | dehydratase | valericigenes | OBV_10870 | | |
| 63 | 4.2.1.— | 2-hydroxyglutaryl-CoA | | hgdA | DSM18026 | 103/182 |
| | | dehydratase | | OBV_10880 | | |
| 64 | 4.2.1.— | 2-hydroxyglutaryl-CoA | | hgdB | DSM18026 | 104/183 |
| | | dehydratase | | OBV_10890 | | |

TABLE 6

| | | | | | Purchased | |
|---|---|---|---|---|---|---|
| NO. | EC | Category | Source Strain | Gene Name | from | Sequence* |
| 65 | 4.2.1.— | 2-hydroxyglutaryl- | Desulfosporosinus | Desor_3092 | DSM765 | 105/184 |
| | | CoA dehydratase | orientis | | | |
| | | | (strain ATCC 19365/ | | | |
| | | | DSM 765/NCIMB 8382/ | | | |
| | | | VKM B-1628) | | | |
| | | | (Desulfotomaculum | | | |
| | | | orientis) | | | |
| 66 | 4.2.1.— | 2-hydroxyglutaryl- | | Desor_3093 | DSM765 | 106/185 |
| | | CoA dehydratase | | | | |
| 67 | 4.2.1.— | 2-hydroxyglutaryl- | | Desor_3091 | DSM765 | 107/186 |
| | | CoA dehydratase | | | | |
| 68 | 4.2.1.— | 2-hydroxyglutaryl- | Peptostreptococcus | BN738_00824 | KCTC5182 | 108/187 |
| | | CoA dehydratase | anaerobius CAG: 621 | | | |
| 69 | 4.2.1.— | 2-hydroxyglutaryl- | | BN738_00823 | KCTC5182 | 109/188 |
| | | CoA dehydratase | | | | |
| 70 | 4.2.1.— | 2-hydroxyglutaryl- | | BN738_00825 | KCTC5182 | 110/189 |
| | | CoA dehydratase | | | | |
| 71 | 4.2.1.— | 2-hydroxyglutaryl- | Chloroflexus aggregans | Cagg_1174 | DSM9485 | 111/190 |
| | | CoA dehydratase | (strain MD-66/DSM 9485) | | | |
| 72 | 4.2.1.17 | 2-hydroxyglutaryl- | Marivirga tractuosa | Ftrac_3721 | KCTC2958 | 112/191 |
| | | CoA dehydratase | (strain ATCC 23168/DSM 4126/ | | | |
| | | | NBRC 15989/NCIMB 1408/ | | | |
| | | | VKMB-1430/H-43) | | | |
| | | | (Microscilla tractuosa) | | | |
| | | | (Flexibacter tractuosus) | | | |
| 73 | 4.2.1.— | 2-hydroxyglutaryl- | Marinithermus | Marky_1278 | DSM14884 | 113/192 |
| | | CoA dehydratase | hydrothermalis | | | |
| | | | (strain DSM 14884/ | | | |
| | | | JCM 11576/T1) | | | |
| 74 | 4.2.1.— | 2-hydroxyglutaryl- | Chitinophaga pinensis | Cpin_6304 | KCTC3412 | 114/193 |
| | | CoA dehydratase | (strain ATCC 43595/ | | | |
| | | | DSM 2588/NCIB 11800/ | | | |
| | | | UQM 2034) | | | |
| 75 | 4.2.1.— | 2-hydroxyglutaryl- | Megasphaera elsdenii | MELS_0744 | KCTC5187 | 115/194 |
| | | CoA dehydratase | DSM 20460 | | | |
| 76 | 4.2.1.— | 2-hydroxyglutaryl- | Megasphaera elsdenii | MELS_0745 | KCTC5187 | 116/195 |
| | | CoA dehydratase | DSM 20460 | | | |
| 77 | 4.2.1.— | 2-hydroxyglutaryl- | Megasphaera elsdenii | MELS_0746 | KCTC5187 | 117/196 |
| | | CoA dehydratase | DSM 20460 | | | |
| 78 | 4.2.1.— | 2-hydroxyglutaryl- | Chloroflexus aurantiacus | Chy400_0108 | DSM635 | 118/197 |
| | | CoA dehydratase | (strain ATCC 29364/ | | | |
| | | | DSM 637/Y-400-fl) | | | |
| 79 | 4.2.1.— | enoyl-CoA | Ruegeria pomeroyi DSS-3 | SP00147 | DSM15171 | 119/198 |
| | | hydrastase | | | | |

In the microorganism, the activity of an enzyme catalyzing conversion of acrylyl-CoA to acrylate may be increased.

The enzyme catalyzing conversion of acrylyl-CoA to acrylate may belong to EC 3.2.1—including EC 3.1.2.4. The enzyme catalyzing conversion of acrylyl-CoA to acrylate may be 3-HP-CoA hydrolase or 3-hydroxyisobutyryl-CoA hydrolase. The enzyme catalyzing conversion of acrylyl-CoA to acrylate may have a higher activity of catalyzing conversion of acrylyl-CoA to acrylate have than the activity of catalyzing the reverse reaction. The enzyme catalyzing conversion of acrylyl-CoA to acrylate may include an amino acid sequence having about 65% or more, for example, 70% or more, about 80% or more, about 85% or more, about 90% or more, about 91% or more, about 92% or more, about 93% or more, about 94% or more, about 95% or more, about 96% or more, about 97% or more, about 98% or more, about 99% or more, or about 100% sequence identity with amino acid sequences of SEQ ID NOS: 199 to 204. A polynucleotide encoding the enzyme catalyzing conversion of acrylyl-CoA to acrylate may encode an amino acid sequence having about 65% or more, for example, 70% or more, about 80% or more, about 85% or more, about 90% or more, about 91% or more, about 92% or more, about 93% or more, about 94% or more, about 95% or more, about 96% or more, about 97% or more, about 98% or more, about 99% or more, or about 100% sequence identity with amino acid sequences of SEQ ID NOS: 199 to 204. A polynucleotide encoding the enzyme catalyzing conversion of acrylyl-CoA to acrylate may have about 95% or more sequence identity with nucleotide sequences of SEQ ID NOS: 205 to 210. The enzyme catalyzing conversion of acrylyl-CoA to acrylate may be at least one of enzymes shown in Table 7. The enzymes shown in Table 7 may be an E3 type. In Table 7, the “Sequence*” refers to amino acid/nucleotide SEQ ID NOs.

TABLE 7

| | | | | | Purchased | |
|---|---|---|---|---|---|---|
| NO. | EC | Category | Source Strain | Gene Name | from | Sequence* |
| 1 | 3.1.2.— | Acyl-CoA thioester | E. coli | yciA | Possessed by | 199/205 |
| | | hydrolase | | | Inventors | |
| 2 | 3.1.2.— | Acyl-CoA thioester | Klebsiella oxytoca | HMPREF9689_01673 | KCTC1686 | 200/206 |
| | | hydrolase | 10-5245 | | | |
| 3 | 3.1.2.— | Acyl-CoA thioester | Cronobacter | yciA | Possessed by | 201/207 |
| | | hydrolase | turicensis | | Inventors | |
| 4 | 3.1.2.— | Acyl-CoA thioester | Citrobacter freundii | D186_20262 | Possessed by | 202/208 |
| | | hydrolase | | | Inventors | |
| 5 | 3.1.2.— | Acyl-CoA thioester | Salmonella enterica | Sel_A1458 | DSM5569 | 203/209 |
| | | hydrolase | | | | |
| 6 | 3.1.2.— | Acyl-CoA thioester | Shigella flexneri | SF123566_2028 | Possessed by | 204/210 |
| | | hydrolase | 1235-66 | | Inventors | |

The microorganism may be a microorganism which is genetically engineered to have an increased expression of the genes of the above enzymes (ALDH, 3-HP-CoA dehydratase, and enzyme catalyzing conversion of acrylyl-CoA to acrylate), for example, an increased expression of the genes of ALDH, and 3-HP-CoA dehydratase, or the genes of ALDH, 3-HP-CoA dehydratase and enzyme catalyzing conversion of acrylyl-CoA to acrylate, in comparison with a cell that is not genetically engineered. When the activity of the enzymes already exists in a parent cell, the expression of the enzymes may be further increased by genetic engineering. When the activity of the enzymes does not exist in a wild-type microorganism, genes encoding the enzymes may be introduced to a parent cell by a genetic engineering method so that the genes may be expressed or overexpressed. The cell that is not genetically engineered refers to a wild-type microorganism or a parent cell from which the microorganism is derived.

Expression or overexpression of the genes of the enzymes may be accomplished by various methods known to this art. For example, expression may be increased by increasing a gene copy number or by using a regulatory material such as an inducer or a repressor. The increase of a copy number may be caused by introduction or amplification of the gene. In other words, the increase of a copy number may be accomplished by introducing an operably linked regulatory factor, a vector including genes of the enzymes, and an expression cassette to a host cell.

Alternatively, increase of activity of the enzymes may be caused by modification of an expression regulatory sequence of the genes. The regulatory sequences may be a promoter sequence or a transcription terminator sequence for expression of the gene.

In addition, the regulatory sequences may be a sequence encoding a motif that may affect gene expression. The motif may be, for example, a secondary structure-stabilization motif, a RNA destabilization motif, a splice-activation motif, a polyadenylation motif, an adenine-rich sequence, or an endonuclease recognition site.

The microorganism may be one selected from the group consisting of bacteria, yeasts, and fungi. For example, the microorganism may be selected from the group consisting of Escherichia, Corynebacterium, and Brevibacterium genera. The cell may be a Corynebacterium genus strain. The microorganism may be one selected from the group consisting of E. coli, Corynebacterium glutamicum, Corynebacterium thermoaminogenes, Brevibacterium flavum, and Brevibacterium lactofermentum. The microorganism may be selected from a genera within the Enterobacteriaceae family other than E. coli.

The microorganism may be a microorganism that produces acrylic acid naturally or a microorganism that is genetically engineered by a recombinant method to produce acrylic acid. In this case, the microorganism may be a microorganism capable of producing acrylic acid from monosaccharides such as glucose, or a glycerol. In addition, the microorganism may have the capability to produce 3-HPA, for example from monosaccharides such as glucose, or a glycerol. The microorganism may have a biochemical pathway forming glycerol from monosaccharides such as glucose. The biochemical pathway may include glycolytic pathway converting monosaccharides such as glucose to dihydroxyacetone phosphate (DHAP), and a pathway converting DHAP to glycerol such as dihydroxyacetone phosphate phosphatase (DHAPP) that catalyzes the conversion of dihydroxyacetone phosphate (DHAP) into dihydroxyacetone (DHA); and glycerol dehydrogenase (GLDH) that catalyzes the conversion of DHA into glycerol. The microorganism may include a polynucleotide encoding dihydroxyacetone phosphate phosphatase (DHAPP) that catalyzes the conversion of dihydroxyacetone phosphate (DHAP) into dihydroxyacetone (DHA); and a polynucleotide encoding glycerol dehydrogenase (GLDH) that catalyzes the conversion of DHA into glycerol. The microorganism may have a biochemical pathway forming 3-HPA from glycerol. The microorganism may include glycerol dehydratase (GDH) that catalyzes the conversion of glycerol into 3-hydroxypropionaldehyde (3-HPA). The microorganism may include a polynucleotide encoding glycerol dehydratase (GDH) that catalyzes the conversion of glycerol into 3-hydroxypropionaldehyde (3-HPA). When the microorganism does not produce acrylic acid naturally, the microorganism may be a microorganism that is genetically engineered to produce acrylic acid. In the microorganism, a gene encoding an enzyme catalyzing a reaction of converting glycerol to 3-HPA may be introduced to have the capability to produce 3-HPA, for example from monosaccharides such as glucose, or a glycerol. The microorganism may be, for example, a strain of Escherichia genus including Escherichia coli. The enzyme catalyzing a reaction of converting glycerol to 3-HPA may be glycerol dehydratase (GDH).

The GDH may include any enzymes catalyzing conversion of glycerol to 3-HPA. The GDH may belong to EC 4.2.1.30 or diol dehydratase (EC 4.2.1.28). The GDH and a nucleotide encoding the same may be derived from Ilyobacter polytropus, Klebsiella pneumoniae, Citrobacter freundii, Clostritidium pasteurianum, Salmonella typhimurium, or Klebsiella oxytoca. In each case, the GDH may comprise three subunits: a large or “a” subunit, a medium or “1” subunit, and a small or “γ” subunit. A gene encoding the large or “α” subunit of GDH may include dhaB1, gldA, and dhaB. A gene encoding the medium or “β” subunit of GDH may include dhaB2, gldB, and dhaC. A gene encoding the small or “γ” subunit of GDH may include dhaB3, gldC, and dhaE. A gene encoding the large or “α” subunit of diol dehydratase may include pduC and pddA. A gene encoding the medium or “β” subunit of diol dehydratase may include pduD and pddB. A gene encoding the small or “γ” subunit of diol dehydratase may include pduE and pddC. The names of genes for GDH and for functions linked with GDH, and the GenBank references were compared in Tables 8 and 9. The GDH may include Ilyobacter polytropus-derived dhaB1, dhaB2, and dhaB3. The Ilyobacter polytropus-derived dhaB1, dhaB2, and dhaB3 may have amino acid sequences of SEQ ID NOS: 211, 212, and 213, respectively. The dhaB1 gene, dhaB2 gene, and dhaB3 gene may encode amino acid sequences of SEQ ID NOS: 211, 212, and 213, respectively. The Ilyobacter polytropus-derived dhaB1 gene, dhaB2 gene, and dhaB3 gene may have sequences of SEQ ID NOS: 214, 215, and 216, respectively.

TABLE 8

| | Gene Function | | | | | | | |
|---|---|---|---|---|---|---|---|---|
| Strain (GenBank | Regulation | | Unknown | | Reactivation | | Unknown | |
| Reference NO.) | Gene | Base Pair | Gene | Base Pair | Gene | Base Pair | Gene | Base Pair |
| K. pneumoniae | | | orf2c | 7116-7646 | orf2b | 6762-7115 | orf2a | 5125-5556 |
| (U30903) | | | | | | | | |
| K. pneumoniae | | | | | GdrB | | | |
| (U60992) | | | | | | | | |
| C. freundii | dhaR | 3746-5671 | orfW | 5649-6179 | orfX | 6180-6533 | orfY | 7736-8164 |
| (U09771) | | | | | | | | |
| C. pasteurianum | | | | | | | | |
| (AF051373) | | | | | | | | |
| C. pasteurianum | | | orfW | 210-731 | orfX | 1-196 | orfY | 746-1177 |
| (AF026270) | | | | | | | | |
| S. typhimurium | | | | | pduH | 8274-8645 | | |
| (AF026270) | | | | | | | | |
| K. oxytoca | | | | | DdrB | 2063-2440 | | |
| (AF017781) | | | | | | | | |
| K. oxytoca | | | | | | | | |
| (AF051373) | | | | | | | | |

TABLE 9

| | Gene Function | | | | | | | |
|---|---|---|---|---|---|---|---|---|
| Strain (GenBank | Dehydratase, α | | Dehydratase, α | | Dehydratase, α | | Reactivation | |
| Reference NO.) | Gene | Base Pair | Gene | Base Pair | Gene | Base Pair | Gene | Base Pair |
| K. pneumoniae | dhaB1 | 3047-4714 | dhaB2 | 2450-2890 | dhaB3 | 2022-2447 | orf2a | 186-2009 |
| (U30903) | | | | | | | | |
| K. pneumoniae | gldA | 121-1788 | gldB | 1801-2382 | gldB | 2388-2813 | gdrA | |
| (U60992) | | | | | | | | |
| C. freundii | dhaB | 8556-10223 | dhaC | 10235-10819 | dhaC | 10822-11250 | orfY | 11261-13072 |
| (U09771) | | | | | | | | |
| C. pasteurianum | dhaB | 84-1748 | dhaC | 1779-2318 | dhaC | 2333-2773 | | 2790-4598 |
| (AF051373) | | | | | | | | |
| C. pasteurianum | | | | | | | orfY | |
| (AF026270) | | | | | | | | |
| S. typhimurium | pduC | 3557-5221 | pduD | 5232-5906 | pduD | 5921-6442 | | 6452-8284 |
| (AF026270) | | | | | | | | |
| K. oxytoca | | | | | | | | 241-2073 |
| (AF017781) | | | | | | | | |
| K. oxytoca | pddA | 121-1785 | pddB | 1796-2470 | pddB | 2485-3006 | | |
| (AF051373) | | | | | | | | |

The GDH may include amino acid sequences having about 65% or more, for example, 70% or more, about 80% or more, about 85% or more, about 90% or more, about 91% or more, about 92% or more, about 93% or more, about 94% or more, about 95% or more, about 96% or more, about 97% or more, about 98% or more, about 99% or more, or about 100% sequence identity with sequences of Ilyobacter polytropus-derived dhaB1, dhaB2, and dhaB3.

The microorganism may further include a polynucleotide encoding glycerol dehydratase reactivase (GDR). Glycerol and diol dehydratase is subject to mechanism-based suicide inactivation by glycerol and some other substrates (Daniel et al., FEMS Microbiol. Rev. 22, 553(1999)). The term “glycerol dehydratase reactivase (GDR)” used herein refers to conversion of a dehydratase incapable of catalyzing a reaction with a target substrate to a dehydratase capable of catalyzing a reaction with a target substrate, repression of dehydratase inhibition, or extension of a useful half-life of a dehydratase enzyme in vivo. The GDR may be at least one of dhaB, gdrA, pduG, and ddrA. In addition, GDR may be at least one of orfX, orf2b, gdrB, pduH, and ddrB.

The GDR may be K. pneumoniae (U60992)-derived gdrA and gdrB having amino sequences of SEQ ID NOS: 217 and 218, respectively. Alternatively, the GDR may be Ilyobacter polytropus-derived gdrA and gdrB having amino sequences of SEQ ID NOS: 219 and 220, respectively. The GDR may include amino acid sequences having about 65% or more, for example, 70% or more, about 80% or more, about 85% or more, about 90% or more, about 91% or more, about 92% or more, about 93% or more, about 94% or more, about 95% or more, about 96% or more, about 97% or more, about 98% or more, about 99% or more, or about 100% sequence identity with amino acid sequences of SEQ ID NOS: 217 to 220, respectively. Genes encoding GdrA and GdrB may respectively have sequences encoding amino acid sequences of SEQ ID NOS: 217 to 220, for examples, respective nucleotide sequences of SEQ ID NOS: 221 to 224.

In the microorganism, at least one of a polynucleotide encoding GDH and a polynucleotide encoding GDR may be expressed at a higher level in comparison with a microorganism that is not genetically engineered. The expression level may be an expression level of an mRNA or a protein. The expression level of a protein may be based on the amount or activity of an expressed protein. The expression level may be increased by about 5% or more, about 10% or more, about 15% or more, about 20% or more, about 30% or more, about 50% or more, about 60% or more, about 70% or more, about 100% or more, about 200% or more, or about 300% or more.

The microorganism may have capability of producing 3-HPA. In the microorganism, the expression increase of at least one of a polynucleotide encoding GDH and a polynucleotide encoding GDR may enable to produce 3-HPA at a higher level in comparison with a microorganism that is not genetically engineered. The production of 3-HPA include intracellular production, secretion after intracellular production, or a combination thereof. The intracellularly produced 3-HPA may be converted to other metabolites such as acrylic acid. The 3-HPA production may be increased by about 5% or more, about 10% or more, about 15% or more, about 20% or more, about 30% or more, about 50% or more, about 60% or more, about 70% or more, about 100% or more, about 200% or more, or about 300% or more.

The expression increase at least one of a polynucleotide encoding GDH and a polynucleotide encoding GDR may be caused by introduction of a polynucleotide encoding a polypeptide, by increase of the copy number of the polypeptide, or by mutation of a regulatory region of the polynucleotide. A polynucleotide which is introduced externally or whose copy number is increased may be endogenous or exogenous. The endogenous gene refers to a gene which has existed on a genetic material included in a microorganism. The exogenous gene refers to a gene which is introduced to a host cell by a method such as integration to a host cell genome. An introduced gene may be homologous or heterologous with the host cell.

In the microorganism, activity of at least one enzyme involved in a pathway of degrading acrylate or converting acrylate to another product may be decreased. In the microorganism, a gene encoding at least one enzyme involved in a pathway of degrading acrylate or converting acrylate to another product may be removed or disrupted.

In addition, the microorganism may further include a pathway of converting acrylate to another product. In the microorganism, production of acrylic acid may be intracellular production or secretion after intracellular production. Therefore, the microorganism may further include a pathway involved in intracellularly producing acrylic acid and converting the produced acrylic acid to another product, for example, an enzyme gene and an expression product thereof. The other product may be acrylate ester.

In the microorganism, a pathway of synthesizing lactate from pyruvate may be inactivated or attenuated. In the microorganism, activity of lactate dehydrogenase (LDH) may be deleted or decreased. The LDH may have activity of catalyzing a reaction of converting pyruvate to lactate. The LDH may be an enzyme classified as EC.1.1.1.27. For example, the LDH may include an amino acid sequence having about 65% or more, for example, 70% or more, about 80% or more, about 85% or more, about 90% or more, about 91% or more, about 92% or more, about 93% or more, about 94% or more, about 95% or more, about 96% or more, about 97% or more, about 98% or more, about 99% or more, or about 100% sequence identity with an amino acid sequence of SEQ ID NO: 225. In the microorganism, a gene encoding LDH may be disrupted or removed. The LDH gene may encode an amino acid sequence having about 65% or more, for example, 70% or more, about 80% or more, about 85% or more, about 90% or more, about 91% or more, about 92% or more, about 93% or more, about 94% or more, about 95% or more, about 96% or more, about 97% or more, about 98% or more, about 99% or more, or about 100% sequence identity with an amino acid sequence of SEQ ID NO: 225.

Another aspect of the present disclosure provides a method of producing acrylate including culturing of the microorganism in a culture medium.

The culturing may be performed according an appropriate culture medium and culture conditions known in this art. The culture medium and culture conditions may be conveniently adjusted according to the selected microorganism. The culturing method may include batch culturing, continuous culturing, fed-batch culturing or a combination thereof. The microorganism may secrete acrylate extracellularly.

The culture medium may include various carbon sources, nitrogen sources, and trace elements. The carbon source may include a carbohydrate such as glucose, sucrose, lactose, fructose, maltose, starch, and cellulose, a lipid such as soybean oil, sunflower oil, castor oil, and coconut oil, a fatty acid such as palmitic acid, stearic acid, and linoleic acid, an organic acid such as acetic acid or a combination thereof. The culturing may be performed by using glucose as a carbon source. The nitrogen source may include an organic nitrogen source such as peptone, yeast extract, meat extract, malt extract, corn steep liquid, and soybean, an inorganic nitrogen source such as urea, ammonium sulfate, ammonium chloride, ammonium phosphate, ammonium carbonate, and ammonium nitrate or a combination thereof. The culture medium may include as a phosphorous source, for example, potassium dihydrogen phosphate, dipotassium phosphate, a sodium-containing salt corresponding to potassium dihydrogen phosphate, and dipotassium phosphate, and a metal salt such as magnesium sulfate and iron sulfate. The culture medium or an individual component may be added to the culturing solution in a batch mode or a continuous mode.

In addition, a compound such as ammonium hydroxide, potassium hydroxide, ammonia, phosphoric acid or sulfuric acid may be added to the microorganism culturing solution in an appropriate mode to adjust pH of the culture solution. In addition, an endoplasmic reticulum such as fatty acid polyglycol ester may be used during the culturing to repress bubble formation.

The culturing may be performed under microaerobic conditions. The term “microaerobic conditions” refers to an amount of oxygen supplied to a culture solution in a situation where air including a smaller amount of oxygen than that of normal atmosphere is in contact with the culture solution. Microaerobic conditions may be formed, for example, by supplying carbon dioxide or nitrogen to atmospheric air at a flow rate of from about 0.1 to about 0.4 vvm, from about 0.2 to about 0.3 vvm, or at about 0.25 vvm. In addition, microaerobic conditions may be a ventilation rate from about 0 to about 0.4 vvm, from about 0.1 to about 0.3 vvm, or from about 0.15 to about 0.25 vvm. The culturing may be performed in a medium including, for example, from about 1 to 20 wt %, from about 1 to about 10 wt %, or from about 2 to about 10 wt % of glycerol.

The method may further include recovering acrylate from a culture solution (e.g., culture medium). The recovery may be performed from cells or a culture solution excluding cells, or from both cells and a culture solution excluding cells. Separation of acrylic acid from a culture solution may be performed by any separation and purification methods known in the art. The recovery may be performed by centrifugation, chromatography, extraction, filtration, precipitation, or a combination thereof.

In one embodiment, the microorganism may further include a pathway of converting acrylate to another product. The method may further include converting the produced acrylate to another product. The other product may be acrylate ester including polyacrylate.

Hereinafter, the present disclosure will be described in further detail with reference to examples. However, these examples are for illustrative purposes only and are not to be construed to limit the scope of the present disclosure.

<Materials and Methods>

Unless otherwise described, the materials and methods described hereinafter were used in Examples.

(1) Preparation of E. coli Cell Having Capability of Producing 3-HPA

An E. coli strain capable of producing 3-HPA, E. coli K12 (DE3) (Δ yqhD Δ ackA-pta/pET-iBAB), was prepared by the following procedures. The strain in which ackA-pta and yqhD genes are deleted was prepared by a method based on Red recombinase expression through the procedures described below. First, to delete ackA-pta, a PCR amplification was performed by using a pKD4 vector (SEQ ID NO: 226) as a template and a primer set of an ackAKF primer (SEQ ID NO: 227) and an ackAKR primer (SEQ ID NO: 228) as primers to obtain an amplification product having homology with two ends of 45 bp ackA-pta. The DNA was introduced to an E. coli K12 (DE3) strain by electroporation to select a strain having resistance to kanamycin (Km^{R}). Then, it was verified that the ackA-pta gene region of the genome of the strain was substituted with a gene providing resistance to kanamycin.

A pCP20 vector (SEQ ID NO: 230) having a gene of Flp recombinase, which is expressed at a high temperature, was introduced to the obtained strain, and the Flp recombinase was expressed to remove the Km^{R} gene inside the genome. Then, a PCR was performed to verify that the ackA-pta gene was deleted and a Km^{R} gene was not included in the obtained strain. Through the same experimental procedures, an amplification product was obtained by performing a PCR by using a pKD4 vector as a template and a primer set of an yqhDKF primer (SEQ ID NO: 231) and an yqhDKR primer (SEQ ID NO: 232) as primers, and the obtained product was introduced to the strain in which the ackA-pta gene was deleted and the a Km^{R} gene was not included. Then, the Km^{R} gene was removed to finally obtain an SH3 strain in which ackA-pta and yqhD genes were deleted.

A pET-iBAB vector was prepared through the procedures described below.

From the genome DNA of Ilyobacter polytropus, genes encoding glycerol dehydratase (GDH) (dhaB1, dhaB2, and dhaB3) (SEQ ID NOS: 214, 215, and 216) and genes encoding glycerol dehydratase reactivase (GDR) (gdrA and gdrB) (SEQ ID NOS: 223 and 224) were obtained. With the dhaB1, dhaB2, and dhaB3 genes, a PCR was performed by using the genome DNA of Ilyobacter polytropus as a template and a primer set of dhaB123_F (SEQ ID NO: 233) and dhaB123_R (SEQ ID NO: 234) as primers to obtain dhaB123 as a single amplification product. With the gdrA and gdrB genes, a PCR was performed by using the genome DNA of Ilyobacter polytropus as a template and a primer set of gdrAB_F (SEQ ID NO: 235) and gdrAB_R (SEQ ID NO: 236) to obtain gdrAB as a single amplification product. The obtained PCR products were treated with BamHI and SacI restriction enzymes and then cloned into a pETDuet™-1 vector (Novagen, Cat. No. 71146-3) to obtain a pET-iBAB vector.

(2) Preparation of E. coli Strain Capable of Producing 3-HPA to which Genes Encoding ALDH and 3-HP-CoA Dehydratase were Introduced

A vector for producing 3-HP-CoA from glycerol through 3-HPA (pET-iBAB-PduP) was prepared through the procedures described below. A PCR amplification was performed by using the pET-iBAB vector as a template and a primer set of iBAB_Up and iBAB_Dn (SEQ ID NOS: 237 and 238) to obtain a linear vector including dhaB123 and gdrAB. The PCR was performed by using Primestar Max (Takara Inc., R045A) by repeating 30 times a cycle including 15 seconds at 95° C., 15 seconds at 50° C., and 2 minutes at 72° C. In addition, a gene encoding CoA acylating aldehyde dehydrogenase (ALDH) (PduP) was obtained from the genome DNA of Lactobacillus reuteri DSM 20016 by performing a PCR amplification using a primer set of pduP_F and pduP_R (SEQ ID NOS: 239 and 240). The obtained PCR product was cloned to the linear vector by using In-Fusion™ HD Cloning Kit (Clontech Laboratories, Inc.). As a result, a pET-iBAB_PduP (pETDuet-1/dhaB_gdrAB_pduP) vector was obtained.

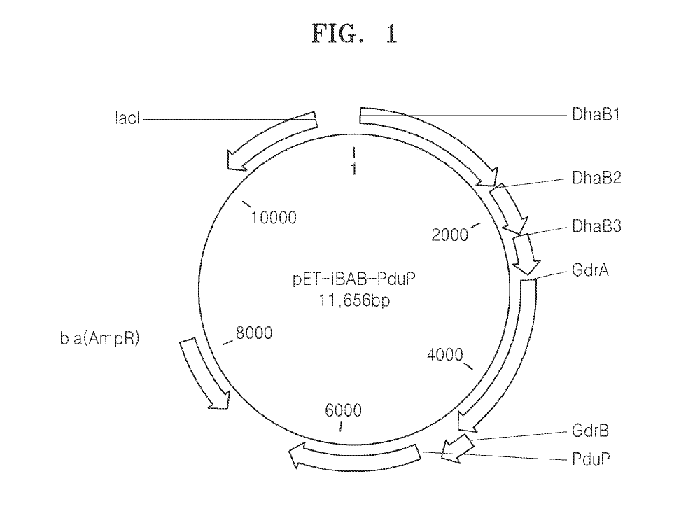

FIG. 1 shows a cleavage map of pET-iBAB_PduP vector.

MELS_{—}1449 gene was introduced to E. coli K12 (DE3) (Δ yqhD Δ ack-pta/pET-iBAB-PduP) as a 3-HP-CoA dehydratase gene.

Specifically, the MELS_{—}1449 gene was amplified by performing a PCR by using the genome of Megasphaera elsdenii strain as a template and a primer set of primers respectively having HindIII and BamHI sites (SEQ ID NOS: 241 and 242). The PCR was performed by using Primestar Max (Takara Inc., R045A) by repeating 30 times a cycle including 15 seconds at 95° C., 15 seconds at 50° C., and 2 minutes at 72° C. The obtained amplification products were digested by using HindIII and BamHI, and the resulting products were linked at the HindIII and BamHI sites of a pACYCDuet™-1 vector (Novagen, cat. no. 71147-3) to prepare pACYC-MDH.

The pET-iBAB-PduP and pACYC-MDH vectors were introduced to an E. coli SH3 strain by electroporation. Specifically, from about 200 to about 300 ng of the two vectors were added to 0.05 mL of an SH3 cell solution prepared for electroporation.

The resulting mixture was added to an electroporation cuvette (Bio-rad Inc., cat. No. 165-2802), and a pulse of 2.5 kV was applied by using Gene Pulser Xcell™ Total System (Bio-rad Inc., cat. No. 165-2660) for transformation. Among the transformed cells, a strain having resistance to both kanamycin antibiotic and chloramphenicol antibiotic was selected to finally prepare an SH3/pET-iBAB-PduP/pACYC-MDH strain.

(3) Preparation of E. coli Strain Having Capability of Producing 3-HPA to which Genes ALDH, 3-HP-CoA Dehydratase, and Enzyme Catalyzing Conversion of Acrylyl-CoA to Acrylate were Introduced

MELS_{—}1449 gene encoding M. elsdenii-derived 3-HP-CoA dehydratase and E. coli-derived CoA hydrolase yciA gene were introduced into E. coli K12 (DE3) (Δ yqhD Δ ack-pta/pET-iBAB-PduP) as genes encoding 3-HP-CoA dehydratase and an enzyme catalyzing conversion of acrylyl-CoA to acrylate.

Specifically, E. coli-derived CoA hydrolase yciA gene was obtained by performing a PCR amplification by using the genome of E. coli (K12 MG1655) as a template and a primer set of yciA_F and yciA_R (SEQ ID NOS: 243 and 244). The amplification products were digested by using BgIII and XhoI restriction enzymes, respectively, and the resulting products were introduced to a pACYC-MDH vector digested by using the same enzymes to prepare a vector for expressing the two genes (pACYC-MDH-yciA).

Next, the pET-iBAB-PduP vector and the pACYC-MDH-YciA vector described in (2) were transformed by electroporation by the same method as preparing the E. coli SH3/pET-iBAB-PduP/pACYC-MDH strain. A strain having resistance to both kanamycin antibiotic and chloramphenicol antibiotic was selected to finally prepare an E. coli SH3/pET-iBAB-PduP/pACYC-MDH-YciA strain.

### Example 1

### Verification of Acrylate Productivity of Microorganism to which Genes Encoding ALDH and 3-HP-CoA Dehydratase Catalyzing Conversion of 3-HP-CoA to Acrylyl-CoA were Introduced

The E. coli SH3, SH3/pET-iBAB-PduP/pACYC-MDH strain, and SH3/pET-iBAB-PduP/pACYC-MDH-YciA strain were respectively inoculated to 20 mL of RM minimal medium (MgSO₄.7H₂O 1.4 g/L, K₂HPO₄ 17.4 g/L, KH₂PO₄ 3 g/L, (NH₄)₂HPO₄ 4 g/L, citric acid 1.7 g/L, ZnCl₂ 0.014 g/L, FeCl₂.4H₂O 0.041 g/L, MnCl₂ 0.015 g/L, CuCl₂ 0.0015 g/L, H₃BO₃ 0.003 g/L, Na₂MoO₄ 0.0025 g/L, vitamin B₁₂ 10 uM, glucose 1.0 g/L, and glycerol 30 g/L) in 250 ml flasks until the optical density at 600 nanometers (OD₆₀₀) value became 0.25, and then cultured at 30° C. until an OD₆₀₀ value became 0.6. Subsequently, 0.03 mM IPTG was added to the culture solution and then cultured at 33° C. for 48 hours. The culturing was performed in 220 mL flasks as shaking culture for 48 hours.

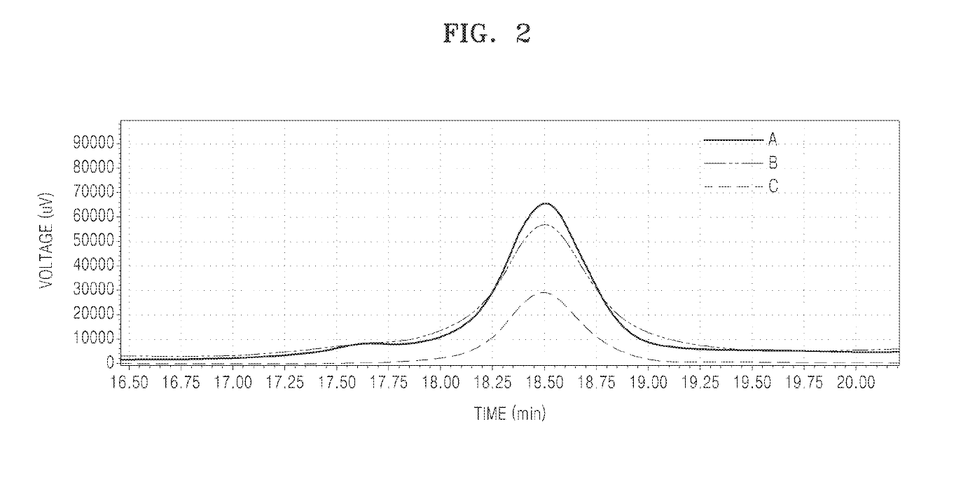

Next, the concentrations of acrylic acid and other organic acids in the culture solution were measured by HPLC. Specifically, after completing the culturing, a part of the culture solution was taken to measure light absorptivity. The culture solution excluding cells was flowed at a flow rate of 0.1 ml/min by using 5 mM of H₂SO₄ aqueous solution into an Aminex HPX-87H column installed at an HPLC (Waters) instrument to which a refractive index detector and a photodiode array detector were attached to verify production of acrylate. The produced acrylate was quantified by a quantity comparison with an acrylate sample (Sigma Aldrich) purified at 210 nm wavelength of a photodiode. The HPLC analysis showed that about 6 mg/L of acrylic acid was produced by culturing for 48 hours the two recombinant E. coli strains to which ALDH gene and 3-HP-CoA dehydratase gene were introduced, the SH3/pET-iBAB-PduP/pACYC-MDH strain and SH3/pET-iBAB-PduP/pACYC-MDH-YciA strain (FIG. 2).

FIG. 2 shows the HPLC analytical results of acrylate in a culture solution, when two recombinant E. coli strains to which ALDH gene and 3-HP-CoA dehydratase gene were introduced were cultured in a glycerol-containing medium. In FIG. 2, A represents an E. coli SH3/pET-iBAB-PduP/pACYC-MDH strain, B represents an SH3/pET-iBAB-PduP/pACYC-MDH-YciA strain, and C represents 2.8 mg/L of acrylate standard sample.

In FIG. 2, the horizontal axis represents the time taken by the culture solution injected to an Aminex HPX-87H column connected with an HPLC to arrive at a photodiode array detector when 5 mM H₂SO₄ aqueous solution was flowed at a rate of 0.1 ml/min, and the vertical axis represents the voltage measured at a 210 nm wavelength range by the photodiode array detector. The acrylate concentration was about 6 mg/L in both of the samples with reference to the acrylate standard sample.

FIG. 3 shows the result of measuring acrylate in a culture solution after culturing an E. coli SH3/pET-iBAB-PduP/pACYC-MDH strain in a fermenter for 48 hours. In FIG. 3, the culturing was performed by inoculating the strain until an OD₆₀₀ value became 0.1 in 600 mL of the RM minimal medium in a 1.5 L fermenter (Biotron) and by culturing at 33° C. at a stirring rate of 600 rpm for 48 hours. As shown in FIG. 3, the SH3/pET-iBAB-PduP/pACYC-MDH strain produced a significantly increased amount of acrylate. The maximum production was 44 mg/L of acrylate at the 40th hour.

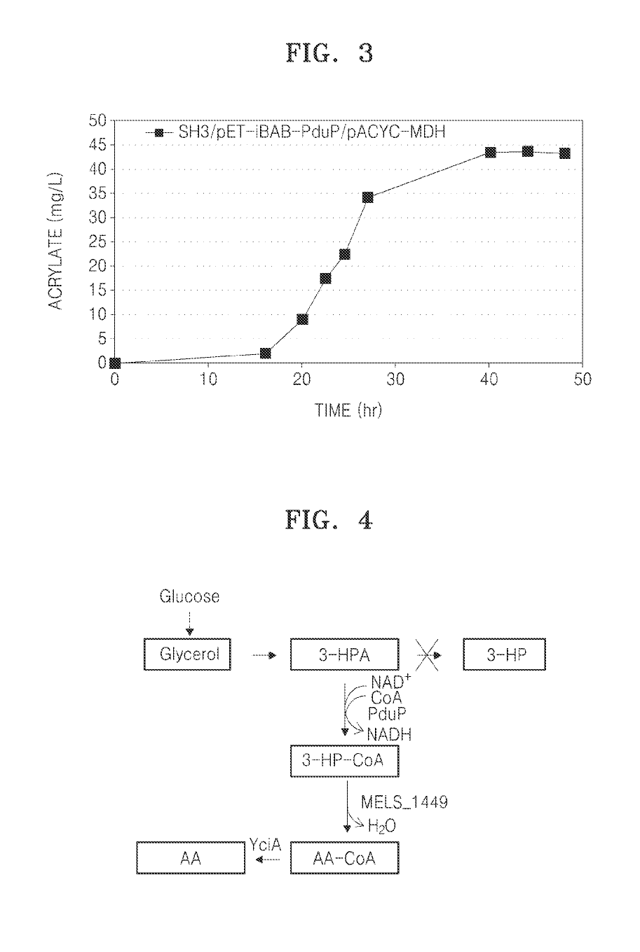

FIG. 4 is a diagram showing an expected pathway of producing acrylic acid from glucose or glycerol in the E. coli of Example 1. In Example 1, it is expected that acrylic acid may be produced through the pathway shown in FIG. 4, but the present disclosure is not limited to a specific mechanism. In FIG. 4, PduP catalyzes conversion of 3-PHA converted from glucose or glycerol to 3-HP-CoA, and MELS_{—}1449 catalyzes conversion of 3-HP-CoA to acrylic acid (AA)-CoA. Conversion of AA-CoA to AA may be catalyzed by an endogenous enzyme, for example, YciA, or by an expression product of an exogenous enzyme gene, for example, an expression product of YciA gene. In E. coli, the YciA gene may be endogenous, and thus AA-CoA may be converted to AA without any exogenous enzymes or genes. As a strain having a pathway of converting a carbon source, for example, glucose or glycerol to 3-HPA, in other words, as a strain having capability of producing 3-HPA, not only the SH3/pET-iBAB-PduP/pACYC-MDH-YciA strain and SH3/pET-iBAB-PduP/pACYC-MDH strain described in Example 1 but also any strains known in the art may be used.

As described above, a microorganism according to one aspect of the present disclosure has increased capability of producing 3-acrylic acid.

According to a method of producing acrylic acid according to another aspect of the present disclosure, acrylic acid may be efficiently produced.

It should be understood that the exemplary embodiments described therein should be considered in a descriptive sense only and not for purposes of limitation. Descriptions of features or aspects within each embodiment should typically be considered as available for other similar features or aspects in other embodiments.

While one or more embodiments of the present disclosure have been described with reference to the figures, it will be understood by those of ordinary skill in the art that various changes in form and details may be made therein without departing from the spirit and scope of the present invention as defined by the following claims.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A genetically engineered microorganism that produces acrylate, wherein the genetically engineered microorganism comprises
a genetic modification that increases CoA acylating aldehyde dehydrogenase (ALDH) activity in catalyzing conversion of 3-hydroxypropionaldehyde (3-HPA) to 3-hydroxy propionyl-CoA (3-HP-CoA); and
a genetic modification that increases 3-HP-CoA dehydratase activity in catalyzing conversion of 3-HP-CoA to acrylyl-CoA;
in comparison with a microorganism of the same type that is not genetically engineered.

2. The microorganism of claim 1, further comprises a genetic modification that increases activity of an enzyme that catalyzes conversion of acrylyl-CoA to acrylate in comparison with a microorganism of the same type that is not genetically engineered.

3. The microorganism of claim 1, wherein the ALDH has an amino acid sequence comprising one of SEQ ID NOs: 1 to 20.

4. The microorganism of claim 1, wherein the ALDH belongs to EC 1.2.1.10, or EC 1.2.1.87.

5. The microorganism of claim 1, wherein the ALDH is propionaldehyde dehydrogenase (pduP).

6. The microorganism of claim 1, wherein the 3-HP-CoA dehydratase has an amino acid sequence comprising one of SEQ ID NOs: 41 to 119.

7. The microorganism of claim 1, wherein the 3-HP-CoA dehydratase belongs to EC 4.2.1.

8. The microorganism of claim 2, wherein the enzyme that catalyzes conversion of acrylyl-CoA to acrylate has an amino acid sequence comprising one of SEQ ID NOs: 199 to 204.

9. The microorganism of claim 2, wherein the enzyme that catalyzes conversion of acrylyl-CoA to acrylate belongs to EC 3.2.1.

10. The microorganism of claim 2, wherein the enzyme that catalyzes conversion of acrylyl-CoA to acrylate is 3-HP-CoA hydrolase or 3-hydroxyisobutyryl-CoA hydrolase.

11. The microorganism of claim 1, wherein the genetically engineered microorganism comprises increased activity of ALDH and 3-HP-CoA dehydratase and the increased activity of ALDH and 3-HP-CoA dehydratase is caused by increased expression of polynucleotides encoding the enzymes as compared to a microorganism of the same type that is not genetically engineered.

12. The microorganism of claim 1, wherein the genetically engineered microorganism comprises exogenous polynucleotides encoding ALDH, 3-HP-CoA dehydratase, and an enzyme catalyzing conversion of acrylyl-CoA to acrylate.

13. The microorganism of claim 1, wherein the microorganism is of the Enterobacteria, Corynebacterium, or Brevibacterium genera.

14. The microorganism of claim 1, wherein a gene encoding at least one enzyme involved in a pathway of degrading acrylate or converting acrylate to another product is deleted or disrupted.

15. The microorganism of claim 1, wherein the genetically engineered microorganism produces 3-HPA.

16. The microorganism of claim 15, wherein the genetically engineered microorganism is E. coli that produces 3-HPA, and comprises an exogenous gene encoding glycerol dehydratase (GDH) and an exogenous gene encoding glycerol dehydratase reactivase (GDR).

17. A method of producing acrylate, the method comprising culturing the microorganism of claim 1 in a culture medium.

18. The method of claim 17, wherein the method further comprises recovering acrylate from the culture.

19. A method of producing a genetically engineered microorganism according to claim 1, the method comprising introducing into a microorganism an exogenous polynucleotide encoding CoA acylating aldehyde dehydrogenase (ALDH), and an exogenous polynucleotide encoding 3-HP-CoA dehydratase.
